(19) United States
(12) Patent Application Publication
(10) Pub. No.: US 2025/0327041 A1
(43) Pub. Date: Oct. 23, 2025

(54) NUCLEOTIDE DELIVERY OF CANCER THERAPY

(71) Applicant: IO Biotech ApS, Copenhagen N (DK)

(72) Inventor: Brian WEINERT, Copenhagen N (DK)

(21) Appl. No.: 18/841,326

(22) PCT Filed: Feb. 23, 2023
(86) PCT No.: PCT/EP2023/054573

§ 371 Date: Aug. 23, 2024

(30) Foreign Application Priority Data
Feb. 24, 2022 (GB) 2202547.2
Apr. 8, 2022 (GB) 2205161.9
Sep. 13, 2022 (GB) 2213372.2

(51) Int. Cl. C12N 9/02; A61K 39/00; C07K 14/495; C07K 14/705; C12N 9/78

(52) U.S. Cl. CPC C12N 9/0069; C07K 14/495; C07K 14/70532; C12N 9/78; A61K 39/00; A61K 2039/53; A61K 2039/55555; A61K 2039/70; C07K 2319/50; C12Y 113/11052; C12Y 305/03001

(57) ABSTRACT

The present invention relates to mRNAs useful in cancer therapies as well as mRNAs for use in a method for the prevention or treatment of cancer in a subject.

### FIELD OF THE INVENTION

The present invention relates to a method for the prevention or treatment of cancer in a subject. The method comprises administering to said subject an mRNA encoding at least one immunogenic peptide fragment of a polypeptide component of an immune system checkpoint, or a vaccine composition comprising said mRNA. Expression of the immunogenic peptide fragment(s) leads to an immune response against the said checkpoint component, reducing its inhibitory effects.

### BACKGROUND TO THE INVENTION

The human immune system is capable of mounting a response against cancerous tumours. Exploiting this response is increasingly seen as one of the most promising routes to treat or prevent cancer. The key effector cell of a long lasting anti-tumour immune response is the activated tumour-specific effector T cell. However, although cancer patients usually have T cells specific for tumour antigens, the activity of these T cells is frequently suppressed by inhibitory factors and pathways, and cancer remains a leading cause of premature deaths in the developed world.

Over the past decade treatments have emerged which specifically target immune system checkpoints. An example of this is Ipilimumab, which is a fully human IgG1 antibody specific for CTLA-4. Treatment of metastatic melanoma with Ipilimumab was associated with an overall response rate of 10.9% and a clinical benefit rate of nearly 30% in a large phase III study and subsequent analyses have indicated that responses may be durable and long lasting. However, these figures still indicate that a majority of the patients do not benefit from treatment, leaving room for improvement.

Accordingly, there exists a need for methods for the prevention or treatment of cancer which augment the T cell anti-tumour response in a greater proportion of patients, but without provoking undesirable effects such as autoimmune disease.

### SUMMARY OF THE INVENTION

The inventors have determined that using mRNA to direct expression by the cells of a patient of at least one immunogenic fragment of a polypeptide component of an immune system checkpoint can provide effective treatment or prevention of cancer.

Advantageously, multiple copies of nucleic acid sequence encoding a given immunogenic peptide fragment can be included in the same mRNA, leading to expression of higher quantities of the peptide in the cellular environment, which may be difficult to achieve with direct administration of the peptide itself. Similarly, nucleic acid sequences encoding different immunogenic peptide fragments can be included in the same mRNA, such that different parts of the same polypeptide component of an immune system checkpoint, or parts of different polypeptide components of the same or different checkpoints, can all be expressed and hence targeted simultaneously. By contrast, it can sometimes be difficult to co-formulate multiple different peptides for direct administration.

The present invention provides:

An mRNA comprising:
An open reading frame (ORF) encoding at least one immunogenic peptide fragment of a polypeptide component of an immune system checkpoint;
A 5′ terminal cap at the 5′ end;
A 5′ untranslated region (UTR) which is included 5′ of the ORF;
A 3′ UTR which is included 3′ of the ORF; and
A 3′ tailing sequence at the 3′ end.

The immune system checkpoint may be selected from any one or more of the following:
The interaction between IDO1 and its substrate;
The interaction between PD1 and PDL1 and/or PD1 and PDL2;
The interaction between Arginase1 or Arginase 2 and its substrate;
The interaction between TDO and its substrate;
The interaction between TGFb1 and its receptors;
The interaction between CTLA4 and CD86 and/or CTLA4 and CD80;
The interaction between B7-H3 and/or B7-H4 and their respective ligands;
The interaction between HVEM and BTLA;
The interaction between GAL9 and TIM3;
The interaction between MHC class I or II and LAG3; and
The interaction between MHC class I or II and KIR

The ORF preferably comprises:
at least one copy of a nucleic acid encoding SEQ ID NO: 2 (IO102) and at least one copy of a nucleic acid encoding SEQ ID NO: 15 (IO103) and/or 16 (IO104.1);
at least one copy of a nucleic acid encoding SEQ ID NO: 2 (IO102) and at least one copy of a nucleic acid encoding SEQ ID NO: 106 (IO112); (IO104.1) and at least one copy of a nucleic acid encoding SEQ ID NO: 106 (IO112);
at least one copy of a nucleic acid encoding SEQ ID NO: 2 (IO102) and at least one copy of a nucleic acid encoding SEQ ID NO: 15 (IO103) and/or 16 (IO104.1) and at least one copy of a nucleic acid encoding SEQ ID NO: 106 (IO112).

The ORF preferably comprises:
at least one copy of SEQ ID NO: 272 (IO102) and at least one copy of SEQ ID NO: 273 (IO103);
at least one copy of SEQ ID NO: 272 (IO102) and at least one copy of SEQ ID NO: 277 (IO112);
at least one copy of SEQ ID NO: 273 (IO103) and at least one copy of SEQ ID NO: 277 (IO112);
at least one copy of SEQ ID NO: 272 (IO102) and at least one copy of SEQ ID NO: 273 (IO103) and at least one copy of SEQ ID NO: 277 (IO112).

Also provided is a vaccine composition comprising the mRNA of the invention, optionally formulated in a lipid nanoparticle composition.

Also provided is a method of treating or preventing a disease comprising administering to a patient in need thereof a therapeutically or prophylactically effective amount of an mRNA or vaccine composition of the invention. Methods and kits for preparing the mRNA or vaccine composition are also provided.

### BRIEF DESCRIPTION OF THE SEQUENCE LISTING

Sequences relevant to the present invention are included in Table X, Table 2 and Table 3.

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “an inhibitor” includes two or more such inhibitors, or reference to “an oligonucleotide” includes two or more such oligonucleotide and the like.

A “subject” as used herein includes any mammal, preferably a human.

A “polynucleotide” is used herein interchangeably with “nucleic acid” to indicate a polymer of nucleosides. Typically a polynucleotide of this invention is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide, unless specified it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e. the succession of letters used as abbreviations for bases) that biochemically characterizes a specific nucleic acid. A polynucleotide sequence presented herein is presented in a 5′ to 3′ direction unless otherwise indicated.

A “polypeptide” is used herein in its broadest sense to refer to a compound of two or more subunit amino acids, amino acid analogs, or other peptidomimetics. The term “polypeptide” may include short peptide sequences and also longer polypeptides and proteins. However, smaller parts of longer polypeptides and proteins are typically described as “peptides” or “peptide fragments” of such longer polypeptides and proteins. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

As used herein, the term “amino acid” refers to either natural and/or unnatural or synthetic amino acids, including both D or L optical isomers, and amino acid analogs and peptidomimetics.

All publications, patents and patent applications cited herein, whether supra or infra, are hereby incorporated by reference in their entirety.

### Immune System Checkpoints and Polypeptide Components Thereof

Effector T cell activation is normally triggered by the T cell receptor recognising antigenic peptide presented by the MHC complex. The type and level of activation achieved is then determined by the balance between signals which stimulate and signals which inhibit the effector T cell response. The term “immune system checkpoint” is used herein to refer to any molecular interaction which alters the balance in favour of inhibition of the effector T cell response. That is, a molecular interaction which, when it occurs, negatively regulates the activation of an effector T cell. Such an interaction might be direct, such as the interaction between a ligand and a cell surface receptor which transmits an inhibitory signal into an effector T cell. Or it might be indirect, such as the blocking or inhibition of an interaction between a ligand and a cell surface receptor which would otherwise transmit an activatory signal into the effector T cell, or an interaction which promotes the upregulation of an inhibitory molecule or cell, or the depletion by an enzyme of a metabolite required by the effector T cell, or any combination thereof.

Examples of immune system checkpoints include:
a. The interaction between IDO1 and its substrate;
b. The interaction between PD1 and PDL1 and/or PD1 and PDL2;
c. The interaction between Arginase1 or Arginase 2 and its substrate;
d. The interaction between TDO and its substrate;
e. The interaction between TGFb1 and its receptors;
f. The interaction between CTLA4 and CD86 and/or CTLA4 and CD80;
g. The interaction between B7-H3 and/or B7-H4 and their respective ligands;
h. The interaction between HVEM and BTLA;
i. The interaction between GAL9 and TIM3;
j. The interaction between MHC class I or II and LAG3; and
k. The interaction between MHC class I or II and KIR

Checkpoint (a), namely the interaction between IDO1 and its substrate, is a preferred checkpoint for the purposes of the present invention. This checkpoint is the metabolic pathway in cells of the immune system requiring the essential amino acid tryptophan. A lack of tryptophan results in the general suppression of effector T cell functions and promotes the conversion of naïve T cells into regulatory (i.e. immunosuppressive) T cells (Tregs). The protein IDO1 is upregulated in cells of many tumours and is responsible for degrading the level of tryptophan. IDO1 is an enzyme that catalyzes the conversion of L-tryptophan to N-formylkynurenine and is thus the first and rate limiting enzyme of tryptophan catabolismthrough the Kynurenine pathway. Therefore, IDO1 (which may be referred to as IDO) is a polypeptide component of an immune system checkpoint which may preferably be targeted in the method of the invention. The full length sequence of IDO is provided in Table X. A preferred mRNA typically includes an ORF encoding at least one immunogenic peptide fragment of IDO. Said fragment may typically comprise or consist of at least 8 or 9 consecutive amino acids of the polypeptide. Said fragment may typically consist of up to 50 consecutive amino acids of IDO1, up to 40 consecutive amino acids of IDO1, up to 30 consecutive amino acids of IDO1, or up to 25 consecutive amino acids of IDO1. The consecutive amino acid sequence of IDO1 may be altered by one or more conservative substitutions, provided the immunogenic properties of the starting sequence are retained. Preferred fragments of IDO are disclosed in WO2009/143843, WO2017/149150, WO2019/101954 and PCT/EP2021/074064 each of which is herein incorporated by reference. Preferred immunogenic peptide fragments of IDO are also shown in Table X. A preferred immunogenic peptide fragment comprises or consists of the sequence of any one of SEQ ID NOs: 2 to 13. The most preferred immunogenic peptide fragment comprises or consists of the sequence of SEQ ID NO: 2 (The peptide consisting of this sequence may be referred to as IO102).

Checkpoint (b), namely the interaction between PD1 and either of its ligands PD-L1 and PD-L2, is another preferred checkpoint for the purposes of the present invention. PD1 is expressed on effector T cells. Engagement with either PD-L1 or PD-L2 results in a signal which downregulates activation. The ligands are expressed by some tumours. PD-L1 in particular is expressed by many solid tumours, including melanoma. These tumours may therefore down regulate immune mediated anti-tumour effects through activation of the inhibitory PD-1 receptors on T cells. By blocking the interaction between PD1 and one or both of its ligands, a checkpoint of the immune response may be removed, leading to augmented anti-tumour T cell responses. Therefore PD1, PD-L1 and PD-L2 are each polypeptide components of an immune system checkpoint which may preferably be targeted in the method of the invention. PD-L1 and PD-L2 are preferred, with PD-L1 most preferred.

The full length sequence of PD-L1 is provided in Table X. A preferred mRNA typically includes an ORF encoding at least one immunogenic peptide fragment of PD-L1. Said fragment may typically comprise or consist of at least 8 or 9 consecutive amino acids of the polypeptide. Said fragment may typically consist of up to 50 consecutive amino acids of PD-L1, up to 40 consecutive amino acids of PD-L1, up to 30 consecutive amino acids of PD-L1, or up to 25 consecutive amino acids of PD-L1. The consecutive amino acid sequence of PD-L1 may be altered by one or more conservative substitutions, provided the immunogenic properties of the starting sequence are retained. Preferred fragments of PD-L1 are disclosed in WO2013/056716, WO2017/220602, WO2017/149150, WO2019/101954 and PCT/EP2021/074064 each of which is herein incorporated by reference. Preferred immunogenic peptide fragments of PD-L1 are also shown in Table X. A preferred immunogenic peptide fragment comprises or consists of the sequence of any one of SEQ ID NOs: 15 to 100. Particularly preferred are an immunogenic peptide fragment comprising or consisting of the sequence of any one of SEQ ID NOs: 15 to 25, most preferably of SEQ ID NO: 15, 16 or 17. The most preferred immunogenic peptide fragment of PD-L1 comprises or consists of the sequence of SEQ ID NO: 15 (The peptide consisting of this sequence may be referred to as IO103).

The full length sequence of PD-L2 is provided in Table X. A preferred mRNA typically includes an ORF encoding at least one immunogenic peptide fragment of PD-L2. Said fragment may typically comprise or consist of at least 8 or 9 consecutive amino acids of the polypeptide. Said fragment may typically consist of up to 50 consecutive amino acids of PD-L2, up to 40 consecutive amino acids of PD-L2, up to 30 consecutive amino acids of PD-L2, or up to 25 consecutive amino acids of PD-L2. The consecutive amino acid sequence of PD-L2 may be altered by one or more conservative substitutions, provided the immunogenic properties of the starting sequence are retained. Preferred fragments of PD-L2 are disclosed in WO2018/077629 which is herein incorporated by reference. Preferred immunogenic peptide fragments of PD-L2 are also shown in Table X. A preferred immunogenic peptide fragment comprises or consists of the sequence of any one of SEQ ID NOs: 102, 103 or 104. The most preferred immunogenic peptide fragment comprises or consists of the sequence of SEQ ID NO: 102.

Checkpoint (c), namely the interaction between Arginase1 or Arginase 2 and its substrate, is another preferred checkpoint for the purposes of the present invention. Arginase1 and 2 are enzymes that catalyses a reaction which converts the amino acid L-arginine into L-ornithine and urea. This depletes the microenvironment of arginine and leads to a suppression of tumor-specific cytotoxic T-cell responses. Increased Arginase activity has been detected in the cancer cells of patients with breast, lung, colon or prostate cancer. It has been shown both in vitro and in vivo that mouse macrophages transfected with a rat Arginase gene promote the proliferation of co-cultured tumour cells. In the clinical setting the induction of Arginase 1 and/or 2 specific immune responses could in addition to the killing of cancer cells support anti-cancer immune responses in general by suppressing the immune suppressive function of Arginase expressing cells especially MDSC and tumor-associated macrophages (TAMs). Therefore Arginase1 and Arginase2 are each polypeptide components of an immune system checkpoint which may preferably be targeted in the method of the invention. Arginase 1 is most preferred.

The full length sequence of Arginase1 is provided in Table X. A preferred mRNA typically includes an ORF encoding at least one immunogenic peptide fragment of Arginase1. Said fragment may typically comprise or consist of at least 8 or 9 consecutive amino acids of the polypeptide. Said fragment may typically consist of up to 50 consecutive amino acids of Arginase1, up to 40 consecutive amino acids of Arginase1, up to 30 consecutive amino acids of Arginase1, or up to 25 consecutive amino acids of Arginase1. The consecutive amino acid sequence of Arginase1 may be altered by one or more conservative substitutions, provided the immunogenic properties of the starting sequence are retained. Preferred fragments of Arginase1 are disclosed in WO2018/065563 and WO2020/064744 each of which is herein incorporated by reference. Preferred immunogenic peptide fragments of Arginase1 are also shown in Table X. A preferred immunogenic peptide fragment comprises or consists of the sequence of any one of SEQ ID NOs: 106 to 158. Particularly preferred are an immunogenic peptide fragment comprising or consisting of the sequence of any one of SEQ ID NOs: 106 to 111, most preferably of SEQ ID NO: 106, 107 or 108. The most preferred immunogenic peptide fragment of Arginase1 comprises or consists of the sequence of SEQ ID NO: 106 (The peptide consisting of this sequence may be referred to as IO112).

The full length sequence of Arginase2 is provided in Table X. A preferred mRNA typically includes an ORF encoding at least one immunogenic peptide fragment of Arginase2. Said fragment may typically comprise or consist of at least 8 or 9 consecutive amino acids of the polypeptide. Said fragment may typically consist of up to 50 consecutive amino acids of Arginase2, up to 40 consecutive amino acids of Arginase2, up to 30 consecutive amino acids of Arginase2, or up to 25 consecutive amino acids of Arginase2. The fragment may comprise or consist of 9-19 consecutive amino acids of Arginase2. The consecutive amino acid sequence of Arginase2 may be altered by one or more conservative substitutions, provided the immunogenic properties of the starting sequence are retained. Preferred fragments of Arginase2 are disclosed in WO2018/065563, WO2020/099582, and GB2202547.2 each of which is herein incorporated by reference. Preferred immunogenic peptide fragments of Arginase2 are also shown in Table X. A preferred immunogenic peptide fragment comprises or consists of the sequence of any one of SEQ ID NOs: 160 to 220. Particularly preferred are an immunogenic peptide fragment comprising or consisting of the sequence of any one of SEQ ID NOs: 160 to 182, most preferably of SEQ ID NO: 160, 161, 162, 163 or 164. The most preferred immunogenic peptide fragment of Arginase2 comprises or consists of the sequence of SEQ ID NO: 160 (The peptide consisting of this sequence may be referred to as A2L2), or a peptide of 9 to 19 consecutive amino acids of Arginase2 which include those of SEQ ID NO: 163 or 164.

Checkpoint (d), namely the interaction between TDO and its substrate, is also a preferred checkpoint for the purposes of the present invention. Although by distinct mechanisms and sharing no sequence homology, both TDO and IDO catalyze the first and rate-limiting step of tryptophan oxidation yielding kynurenine. Thus checkpoint (d) is similar to checkpoint (a) and TDO is a polypeptide component of an immune system checkpoint which may preferably be targeted in the method of the invention. The full length sequence of TDO is provided in Table X. A preferred mRNA typically includes an ORF encoding at least one immunogenic peptide fragment of TDO. Said fragment may typically comprise or consist of at least 8 or 9 consecutive amino acids of the polypeptide. Said fragment may typically consist of up to 50 consecutive amino acids of TDO, up to 40 consecutive amino acids of TDO, up to 30 consecutive amino acids of TDO, or up to 25 consecutive amino acids of TDO. The consecutive amino acid sequence of TDO may be altered by one or more conservative substitutions, provided the immunogenic properties of the starting sequence are retained. Preferred fragments of TDO are disclosed in WO2016/041560 which is herein incorporated by reference. Preferred immunogenic peptide fragments of TDO are also shown in Table X. A preferred immunogenic peptide fragment comprises or consists of the sequence of any one of SEQ ID NOs: 222 to 238. The most preferred immunogenic peptide fragment comprises or consists of up to 25 consecutive amino acids of TDO which include at least the sequence of any one of SEQ ID NOs: 222 to 238.

Checkpoint (e), namely the interaction between TGFb and its receptors, is also a preferred checkpoint for the purposes of the present invention. TGFb is a multifunctional cytokine with a key role in the regulation of the immune system. There are four isoforms, of which isoform 1 (TGFb1) is particularly important in T-cell immunity. In the context of cancer, TGFb1 disarms various immune cells like cytotoxic T-cells (CTLs), tumor-associated neutrophils and Natural Killer (NK) cells. It also contributes to tumor vascularization and metastasis. Consequently, TGFb1 is a key inhibitory molecule in the tumor microenvironment (TME), contributing to a down-regulation of the immune system's anti-tumor machinery and enabling immune-evasion by cancer cells. Therefore TGFb1 is a polypeptide component of an immune system checkpoint which may preferably be targeted in the method of the invention. TGFb1 is a dimeric cytokine which shares a cysteine knot structure connected together by intramolecular disulfide bonds. TGFb1 is synthesized as a monomeric 390-amino acid precursor protein, which is referred to interchangeably as: TGFb1 pre-protein; TGFb1 precursor; full-length TGFb1; pre-pro-TGFb1. The TGFb1 pre-protein monomer has a molecular weight of about 25 kDa. The TGFb1 protein monomer has three distinct domains: the signal peptide (SP: amino acids 1-29), the latency associated peptide (LAP: amino acids 30-278) and the mature peptide (mature TGFb1: amino acids 279-390). The sequences of full length TGFb1, TGFb1 SP, TGFb1 LAP and mature TGFb1 are shown in Table X. A preferred mRNA typically includes an ORF encoding at least one immunogenic peptide fragment of full length TGFb1, TGFb1 SP, TGFb1 LAP or mature TGFb1. Said fragment may typically comprise or consist of at least 8 or 9 consecutive amino acids of the polypeptide. Said fragment may typically consist of up to 50 consecutive amino acids of the polypeptide, up to 40 consecutive amino acids of the polypeptide, up to 30 consecutive amino acids of the polypeptide, or up to 25 consecutive amino acids of the polypeptide. The consecutive amino acid sequence of the polypeptide may be altered by one or more conservative substitutions, provided the immunogenic properties of the starting sequence are retained. Preferred fragments of the polypeptide are disclosed in WO2020/245264 which is herein incorporated by reference. Preferred immunogenic peptide fragments of TGFb1 are also shown in Table X. A preferred immunogenic peptide fragment comprises or consists of the sequence of any one of SEQ ID NOs: 243 to 271, preferably SEQ ID NOs: 243 to 247, most preferably SEQ ID NOs: 243 or 244. The most preferred immunogenic peptide fragment comprises or consists of the sequence of SEQ ID NO: 243. (The peptide consisting of this sequence may be referred to as TGFb15).

Another preferred checkpoint for the purposes of the present invention is checkpoint (f), namely the interaction between the T cell receptor CTLA-4 and its ligands, the B7 proteins (B7-1 and B7-2). CTLA-4 is ordinarily upregulated on the T cell surface following initial activation, and ligand binding results in a signal which inhibits further/continued activation. CTLA-4 competes for binding to the B7 proteins with the receptor CD28, which is also expressed on the T cell surface but which upregulates activation. Thus, by blocking the CTLA-4 interaction with the B7 proteins, but not the CD28 interaction with the B7 proteins, one of the normal check points of the immune response may be removed, leading to augmented anti-tumour T cell responses. Therefore CTLA4 and its ligands are examples of polypeptide components of an immune system checkpoint which may preferably be targeted in the method of the invention. A preferred mRNA typically includes an ORF encoding at least one immunogenic peptide fragment of CTLA4 or one of its ligands.

The same applies to the polypeptide components of any one of checkpoints (g) to (k). A preferred mRNA typically includes an ORF encoding at least one immunogenic peptide fragment of a polypeptide component of any one of checkpoints (g) to (k).

In some embodiments, the ORF encodes at least one immunogenic polypeptide fragment of checkpoints (a) and (b). In some embodiments, the ORF encodes at least one immunogenic polypeptide fragment of checkpoints (a) and (c). In some embodiments, the ORF encodes at least one immunogenic polypeptide fragment of checkpoints (a) and (d). In some embodiments, the ORF encodes at least one immunogenic polypeptide fragment of checkpoints (a) and (e). In some embodiments, the ORF encodes at least one immunogenic polypeptide fragment of checkpoints (a) and (f). In some embodiments, the ORF encodes at least one immunogenic polypeptide fragment of checkpoints (a) and (g). In some embodiments, the ORF encodes at least one immunogenic polypeptide fragment of checkpoints (a) and (h). In some embodiments, the ORF encodes at least one immunogenic polypeptide fragment of checkpoints (a) and (i). In some embodiments, the ORF encodes at least one immunogenic polypeptide fragment of checkpoints (a) and (j). In some embodiments, the ORF encodes at least one immunogenic polypeptide fragment of checkpoints (a) and (k). In some embodiments, the ORF encodes at least one immunogenic polypeptide fragment of checkpoints (b) and (c). In some embodiments, the ORF encodes at least one immunogenic polypeptide fragment of checkpoints (b) and (d). In some embodiments, the ORF encodes at least one immunogenic polypeptide fragment of checkpoints (b) and (e). In some embodiments, the ORF encodes at least one immunogenic polypeptide fragment of checkpoints (b) and (f). In some embodiments, the ORF encodes at least one immunogenic polypeptide fragment of checkpoints (b) and (g). In some embodiments, the ORF encodes at least one immunogenic polypeptide fragment of checkpoints (b) and (h). In some embodiments, the ORF encodes at least one immunogenic polypeptide fragment of checkpoints (b) and (i). In some embodiments, the ORF encodes at least one immunogenic polypeptide fragment of checkpoints (b) and (j). In some embodiments, the ORF encodes at least one immunogenic polypeptide fragment of checkpoints (b) and (k). In some embodiments, the ORF encodes at least one immunogenic polypeptide fragment of checkpoints (c) and (d). In some embodiments, the ORF encodes at least one immunogenic polypeptide fragment of checkpoints (c) and (e). In some embodiments, the ORF encodes at least one immunogenic polypeptide fragment of checkpoints (c) and (f). In some embodiments, the ORF encodes at least one immunogenic polypeptide fragment of checkpoints (c) and (g). In some embodiments, the ORF encodes at least one immunogenic polypeptide fragment of checkpoints (c) and (h). In some embodiments, the ORF encodes at least one immunogenic polypeptide fragment of checkpoints (c) and (i). In some embodiments, the ORF encodes at least one immunogenic polypeptide fragment of checkpoints (c) and (j). In some embodiments, the ORF encodes at least one immunogenic polypeptide fragment of checkpoints (c) and (k). In some embodiments, the ORF encodes at least one immunogenic polypeptide fragment of checkpoints (d) and (e). In some embodiments, the ORF encodes at least one immunogenic polypeptide fragment of checkpoints (d) and (f). In some embodiments, the ORF encodes at least one immunogenic polypeptide fragment of checkpoints (d) and (g). In some embodiments, the ORF encodes at least one immunogenic polypeptide fragment of checkpoints (d) and (h). In some embodiments, the ORF encodes at least one immunogenic polypeptide fragment of checkpoints (d) and (i). In some embodiments, the ORF encodes at least one immunogenic polypeptide fragment of checkpoints (d) and (j). In some embodiments, the ORF encodes at least one immunogenic polypeptide fragment of checkpoints (d) and (k). In some embodiments, the ORF encodes at least one immunogenic polypeptide fragment of checkpoints (e) and (f). In some embodiments, the ORF encodes at least one immunogenic polypeptide fragment of checkpoints (e) and (g). In some embodiments, the ORF encodes at least one immunogenic polypeptide fragment of checkpoints (e) and (h). In some embodiments, the ORF encodes at least one immunogenic polypeptide fragment of checkpoints (e) and (i). In some embodiments, the ORF encodes at least one immunogenic polypeptide fragment of checkpoints (e) and (j). In some embodiments, the ORF encodes at least one immunogenic polypeptide fragment of checkpoints (e) and (k). In some embodiments, the ORF encodes at least one immunogenic polypeptide fragment of checkpoints (f) and (g). In some embodiments, the ORF encodes at least one immunogenic polypeptide fragment of checkpoints (f) and (h). In some embodiments, the ORF encodes at least one immunogenic polypeptide fragment of checkpoints (f) and (i). In some embodiments, the ORF encodes at least one immunogenic polypeptide fragment of checkpoints (f) and (j). In some embodiments, the ORF encodes at least one immunogenic polypeptide fragment of checkpoints (f) and (k). In some embodiments, the ORF encodes at least one immunogenic polypeptide fragment of checkpoints (g) and (h). In some embodiments, the ORF encodes at least one immunogenic polypeptide fragment of checkpoints (g) and (i). In some embodiments, the ORF encodes at least one immunogenic polypeptide fragment of checkpoints (g) and (j). In some embodiments, the ORF encodes at least one immunogenic polypeptide fragment of checkpoints (g) and (k). In some embodiments, the ORF encodes at least one immunogenic polypeptide fragment of checkpoints (h) and (i). In some embodiments, the ORF encodes at least one immunogenic polypeptide fragment of checkpoints (h) and (j). In some embodiments, the ORF encodes at least one immunogenic polypeptide fragment of checkpoints (h) and (k). In some embodiments, the ORF encodes at least one immunogenic polypeptide fragment of checkpoints (i) and (j). In some embodiments, the ORF encodes at least one immunogenic polypeptide fragment of checkpoints (i) and (k). In some embodiments, the ORF encodes at least one immunogenic polypeptide fragment of checkpoints (j) and (k).

mRNA Molecules

The invention relates to an mRNA comprising:
An open reading frame (ORF) encoding at least one immunogenic peptide fragment of a polypeptide component of an immune system checkpoint;
A 5′ terminal cap at the 5′ end;
A 5′ untranslated region (UTR) which is included 5′ of the ORF;
A 3′ UTR which is included 3′ of the ORF; and
A 3′ tailing sequence at the 3′ end.

In the mRNA sequences encoding an immunogenic fragment may each be interspersed by a cleavage sensitive site.

The ORF may include multiple copies of each sequence encoding a different immunogenic peptide fragment, optionally at least 2, 3, 4, 5, 10, 15, 20, 25, 30, 35, 40, 45, 50 or more copies of each said sequence, and preferably wherein the ORF encodes at least 2, 3, 4, 5, 10 or more different immunogenic peptide fragments. In some embodiments, different immunogenic peptide fragments are different parts of the same immune system checkpoint component polypeptide. In some embodiments, different immunogenic peptide fragments are fragments of different immune system checkpoint component polypeptides. In some embodiments, different immunogenic peptide fragments are both different parts of the same immune system checkpoint component polypeptide and fragments of different immune system checkpoint component polypeptides.

Preferred mRNA encoding the fragments of immune system checkpoint component polypeptides are shown in Table 2. A preferred mRNA comprises an ORF consisting of or comprising the sequence of any one of SEQ ID NOs: 272-281.

The mRNA may be described as a mRNA vaccine against cancer, or a mRNA cancer vaccine. mRNA vaccines are described in International Patent Application No. WO2015/164674 herein incorporated by reference in its entirety. The mRNA cancer vaccines of the invention may be compositions, including pharmaceutical compositions. The invention also encompasses methods for the preparation, manufacture, formulation, and/or use of mRNA cancer vaccines.

The generation and delivery of immunogenic peptide fragments so that they are presented effectively on MHC Molecules in order to elicit a desired immune response in an individual can be challenging. In some embodiments the mRNA of the invention solves this problem by leading to the expression of polypeptides comprising multiple immunogenic peptide fragments preferably interspersed with cleavage sites recognised by proteases that are abundant in Antigen Presenting Cells (APCs). These methods mimic antigen processing and may lead to a more effective antigen presentation than can be achieved with peptide antigens. The fact that the immunogenic peptide fragments are expressed from RNA as intracellular peptides may provide advantages over delivery as exogenous peptides. The RNA is delivered intra-cellularly and expresses the epitopes in proximity to the appropriate cellular machinery for processing the epitopes such that they will be recognized by the appropriate immune cells. Additionally, a targeting sequence may allow more specificity in the delivery of the peptide epitopes. For example, a C-terminus Ubiquitin Ligase targeting protein (FBox Protein) may be used to target the polypeptide processing to the proteasome and more closely mimic the MHC processing. The constructs of the invention also may include linkers such as proteolytic cleavage sites optimized for APCs. These proteolytic sites provide an advantage because they enhance the processing of the peptides in APCs. When the mRNA cancer vaccine is delivered to a cell, the mRNA will be processed into a polypeptide by the intracellular machinery which can then process the polypeptide into the immunogenic peptide fragments capable of stimulating a desired immune response.

In some embodiments, the mRNA cancer vaccine encodes multiple immunogenic peptide fragments. This may be described as a poly-epitopic mRNA vaccine because each encoded immunogenic peptide fragment comprises at least one epitope. The RNA sequences that code for the immunogenic peptide fragments may be interspersed by sequences that code for amino acid sequences recognized by proteolytic enzymes. Thus, in some embodiments an mRNA cancer vaccine is an mRNA having an open reading frame encoding a propeptide, since the encoded polypeptide sequence includes multiple immunogenic peptide fragments linked together either directly or through a linker such as a cleavage sensitive site. An exemplary propeptide has the following peptide sequence:

Tₘ - Yₒ- (X1-Yo- X2-Yo- . . . Xn)- Yₒ - Tₘ

### Where:

T is a targeting sequence and m=0-1. A targeting sequence may be included at either the N terminus, the C terminus, or both ends of the central peptide region. If a polypeptide has more than one targeting sequence, those sequences may be the same or different.
X_{1, 2} etc are each independently an immunogenic peptide fragment sequence, and n=0-1000. Each immunogenic peptide fragment sequence designated by an X may represent a unique immunogenic peptide fragment sequence in the propeptide or it may refer to a copy of a immunogenic peptide fragment sequence. Thus, the propeptide encoded by the mRNA may be composed of multiple immunogenic peptide fragment sequences each of which is unique and/or it may include more than 1 copy of each unique immunogenic peptide fragment sequence(s). In some embodiments a propeptide may have at least 2, at least 3, at least 4, at least 5, at least 10, at least 20, at least 30, at least 40, at least 50, or more copies of each unique immunogenic peptide fragment sequence. Preferably the propeptide has at least 2, 3, 4, 5, 10 or more different immunogenic peptide fragment sequences.
Y is a linker sequence, preferably a cleavage sensitive sequence, and o=0-5. Each immunogenic peptide fragment sequence may optionally have one or more linkers, optionally cleavage sensitive sites adjacent to the N and/or C terminal end. In a multiepitope design, two or more of the immunogenic peptide fragment sequences may have a cleavage sensitive site between them. Alternatively two or more of the immunogenic peptide fragment sequences may be connected directly to one another or through a linker that is not a cleavage sensitive site. The targeting sequence may also be connected to the immunogenic peptide fragment sequence through a cleavage sensitive site or it may be connected directly to the immunogenic peptide fragment sequence through a linker that is not a cleavage sensitive site.

The invention primarily relates to mRNA encoding at least one immunogenic peptide fragment of a polypeptide component of an immune system checkpoint. Preferred RNA sequences are shown in Table 2. However the mRNA sequences of the invention may be replaced with corresponding “counterpart” DNA sequences. DNA sequences may be either single- and double-stranded forms (and complements of each single-stranded molecule). Suitable “counterpart” DNA sequences are shown in Table 3.

In some embodiments, the vaccine compositions provided herein comprise two or more mRNA polynucleotides, each encoding a different immunogenic peptide fragment. In some embodiments, the vaccine compositions comprise two, three, or four mRNA polynucleotides.

In some embodiments, the vaccine composition comprises a first mRNA polynucleotide encoding a first immunogenic polypeptide fragment and a second mRNA polynucleotide encoding a second immunogenic polypeptide fragment. In some embodiments, the first and second immunogenic polypeptide fragments are fragments of the proteins as follows:

| First fragment | Second fragment |
|---|---|
| IDO | PD1 |
| IDO | PDL1 |
| IDO | Arginase1 |
| IDO | Arginase2 |
| IDO | TDO |
| IDO | TFGβ |
| PD1 | Arginase1 |
| PD1 | Arginase2 |
| PDL1 | Arginase1 |
| PDL1 | Arginase2 |
| PD1 | TDO |
| PDL1 | TDO |
| PD1 | TFGβ |
| PDL1 | TFGβ |
| Arginase1 | TDO |
| Arginase2 | TDO |
| Arginase1 | TFGβ |
| Arginase2 | TFGβ |
| TDO | TFGβ |

In some embodiments, the first and second immunogenic polypeptide fragments are up to 50 consecutive amino acids of the proteins according to the table above.

In some embodiments, the first and second immunogenic polypeptide fragments comprise or consist of the amino acid sequences according to the following table:

| First fragment | Second fragment |
|---|---|
| SEQ ID NOs: 2-13 | SEQ ID NOs: 15-100 |
| SEQ ID NOs: 2-13 | SEQ ID NOs: 102-104 |
| SEQ ID NO: 2 | SEQ ID NO: 15 |
| SEQ ID NO: 2 | SEQ ID NO: 16 |
| SEQ ID NO: 2 | SEQ ID NO: 102-104 |
| SEQ ID NOs: 2-13 | SEQ ID NO: 106-158 |
| SEQ ID NOs: 2-13 | SEQ ID NO: 106 |
| SEQ ID NOs: 2-13 | SEQ ID NOs: 160-220 |
| SEQ ID NOs: 2-13 | SEQ ID NO: 160 |
| SEQ ID NO: 2 | SEQ ID NOs: 106-158 |
| SEQ ID NO: 2 | SEQ ID NO: 106 |
| SEQ ID NO: 2 | SEQ ID NOs: 160-220 |
| SEQ ID NO: 2 | SEQ ID NO: 160 |
| SEQ ID NOs: 2-13 | SEQ ID NOs: 222-238 |
| SEQ ID NO: 2 | SEQ ID NOs: 222-238 |
| SEQ ID NOs: 2-13 | SEQ ID NOs: 240 to 271 |
| SEQ ID NO: 2 | SEQ ID NOs: 240 to 271 |
| SEQ ID NOs: 15 to 100 | SEQ ID NOs: 106 to 158 |
| SEQ ID NOs: 15 to 100 | SEQ ID NO: 106 |
| SEQ ID NO: 15 | SEQ ID NOs: 106 to 158 |
| SEQ ID NO: 15 | SEQ ID NO: 106 |
| SEQ ID NO: 16 | SEQ ID NOs: 106 to 158 |
| SEQ ID NO: 16 | SEQ ID NO: 106 |
| SEQ ID NOs: 15 to 100 | SEQ ID NOs: 160 to 220 |
| SEQ ID NOs: 15 to 100 | SEQ ID NO: 160 |
| SEQ ID NO: 15 | SEQ ID NOs: 160 to 220 |
| SEQ ID NO: 15 | SEQ ID NO: 160 |
| SEQ ID NO: 16 | SEQ ID NOs: 160 to 220 |
| SEQ ID NO: 16 | SEQ ID NO: 160 |
| SEQ ID NOs: 102-104 | SEQ ID NOs: 160 to 220 |
| SEQ ID NOs: 102-104 | SEQ ID NO: 160 |
| SEQ ID NOs: 15 to 100 | SEQ ID NOs: 222-238 |
| SEQ ID NO: 15 | SEQ ID NOs: 222-238 |
| SEQ ID NO: 16 | SEQ ID NOs: 222-238 |
| SEQ ID NOs: 102-104 | SEQ ID NOs: 222-238 |
| SEQ ID NOs: 15 to 100 | SEQ ID NOs: 240-271 |
| SEQ ID NO: 15 | SEQ ID NOs: 240-271 |
| SEQ ID NO: 16 | SEQ ID NOs: 240-271 |
| SEQ ID NO: 102-104 | SEQ ID NOs: 240-271 |
| SEQ ID NOs: 106 to 158 | SEQ ID NOs: 222-238 |
| SEQ ID NO: 106 | SEQ ID NOs: 222-238 |
| SEQ ID NOs: 160 to 220 | SEQ ID NOs: 222-238 |
| SEQ ID NO: 160 | SEQ ID NOs: 222-238 |
| SEQ ID NOs: 106 to 158 | SEQ ID NOs: 240 to 271 |
| SEQ ID NO: 106 | SEQ ID NOs: 240 to 271 |
| SEQ ID NOs: 160 to 220 | SEQ ID NOs: 240 to 271 |
| SEQ ID NO: 160 | SEQ ID NOs: 240 to 271 |
| SEQ ID NOs: 222 to 238 | SEQ ID NOs: 240 to 271 |

In some embodiments, the vaccine composition comprises a first mRNA polynucleotide encoding a first immunogenic polypeptide fragment, a second mRNA polynucleotide encoding a second immunogenic polypeptide fragment, and a third mRNA polynucleotide encoding a third immunogenic peptide fragment of a polypeptide component of an immune system checkpoint.

In some embodiments, the first immunogenic polypeptide fragment is IDO; and preferably wherein the immunogenic peptide fragment thereof is up to 50 consecutive amino acids of IDO, optionally comprising or consisting of the sequence of any one of SEQ ID NOs: 2 to 13, preferably SEQ ID NO: 2; the second immunogenic polypeptide fragment is PDL1 or PDL2, preferably PDL1; and preferably wherein the immunogenic peptide fragment thereof is up to 50 consecutive amino acids of PDL1, optionally comprising or consisting of the sequence of any one of SEQ ID NOs: 15 to 100, preferably SEQ ID NOs: 15 or 16; or alternatively PDL2, and preferably wherein the immunogenic peptide fragment thereof is up to 50 consecutive amino acids of PDL2, optionally comprising or consisting of the sequence of any one of SEQ ID NOs: 102 to 104; and the third immunogenic polypeptide fragment is Arginase1 or Arginase2, preferably Arginase1; and preferably wherein the immunogenic peptide fragment thereof is up to 50 consecutive amino acids of Arginase1, optionally comprising or consisting of the sequence of any one of SEQ ID NOs: 106 to 158, preferably SEQ ID NO: 106; or alternatively Arginase2, and preferably wherein the immunogenic peptide fragment thereof is up to 50 consecutive amino acids of Arginase2, comprising or consisting of the sequence of any one of SEQ ID NOs: 160 to 220; preferably SEQ ID NO: 150.

In some embodiments, the vaccine composition comprises a first mRNA polynucleotide encoding a first immunogenic polypeptide fragment and a second mRNA polynucleotide encoding a second immunogenic polypeptide fragment. In some embodiments, the first polynucleotide comprises at least one copy of a nucleic acid encoding SEQ ID NO: 2 (IO102) and the second polynucleotide comprises at least one copy of a nucleic acid encoding SEQ ID NO: 15 (IO103) and/or 16 (IO104.1). In some embodiments, the first polynucleotide comprises at least one copy of a nucleic acid encoding SEQ ID NO: 2 (IO102) and the second polynucleotide comprises at least one copy of a nucleic acid encoding SEQ ID NO: 106 (IO112). In some embodiments, the first polynucleotide comprises at least one copy of a nucleic acid encoding SEQ ID NO: 15 (IO103) and/or 16 (IO104.1) and the second polynucleotide comprises at least one copy of a nucleic acid encoding SEQ ID NO: 106 (IO112).

In some embodiments, the vaccine composition comprises a first mRNA polynucleotide encoding a first immunogenic polypeptide fragment, a second mRNA polynucleotide encoding a second immunogenic polypeptide fragment, and a third mRNA polynucleotide encoding a third immunogenic peptide fragment of a polypeptide component of an immune system checkpoint. In some embodiments, the first polynucleotide comprises at least one copy of a nucleic acid encoding SEQ ID NO: 2 (IO102) and the second polynucleotide comprises at least one copy of a nucleic acid encoding SEQ ID NO: 15 (IO103) and/or 16 (IO104.1) and the third polynucleotide comprises at least one copy of a nucleic acid encoding SEQ ID NO: 106 (IO112).

In some embodiments, the first, second, and/or third polynucleotide each comprise one or more of SEQ ID NO: 272, 273, 274, 275, 276, 277, 278, 279, 280 and 281. In some embodiments, the first polynucleotide comprises at least one copy of SEQ ID NO: 272 (IO102) and the second polynucleotide comprises at least one copy of SEQ ID NO: 273 (IO103). In some embodiments, the first polynucleotide comprises at least one copy of SEQ ID NO: 272 (IO102) and the second polynucleotide comprises at least one copy of SEQ ID NO: 277 (IO112). In some embodiments, the first polynucleotide comprises one copy of SEQ ID NO: 273 (IO103) and the second polynucleotide comprises at least one copy of SEQ ID NO: 277 (IO112). In some embodiments, the first polynucleotide comprises one copy of SEQ ID NO: 272 (IO102) and the second polynucleotide comprises at least one copy of SEQ ID NO: 273 (IO103), and the third polynucleotide comprises at least one copy of SEQ ID NO: 277 (IO112).

### Targeting Sequences

The mRNA may encode one or more targeting sequence. This may be an endosomal targeting sequence, for example a portion of the transmembrane domain of lysosome associated membrane protein (LAMP-1) or a portion of the transmembrane domain of invariant chain (Ii). The targeting sequence may be a ubiquitination signal that is attached at either or both ends of the encoded peptide. In other embodiments, the targeting sequence is a ubiquitination signal that is attached at an internal site of the encoded peptide and/or to either end. Thus, the RNA may include a nucleic acid sequence encoding a ubiquitination signal at either or both ends of the nucleotides encoding the immunogenic peptide fragment(s).

Ubiquitination, a post-translational modification, is the process of attaching ubiquitin to a substrate target protein. A ubiquitination signal is a peptide sequence which enables the targeting and processing of a peptide to one or more proteasomes. By targeting and processing the peptide through the use of a ubiquitination signal the intracellular processing of the peptide can more closely recapitulate antigen processing in Antigen Presenting Cells (APCs). The number of ubiquitins added to an antigen can enhance the efficacy of the processing step. For instance, in polyubiquitination, additional ubiquitin molecules are added after the first has been attached to the peptide. The resulting ubiquitin chain is created by the linking of the glycine residue of the ubiquitin molecule to a lysine of the ubiquitin bound to the peptide. Each ubiquitin contains seven lysine residues and an N-terminal that can serve as sites for ubiquitination. When four or more ubiquitin molecules are attached to a lysine residue on the peptide antigen, the 26S proteasome recognizes the complex, internalizes it, and degrades the protein into small peptides.

### Cleavage Sensitive Sequences

The immunogenic peptide fragment sequences may be connected in some embodiments by a cleavage sensitive site. A cleavage sensitive site is a peptide which is susceptible to cleavage by an enzyme or protease. These sites are also called protease cleavage sites. Preferably the protease is an intracellular enzyme. The protease may be a serine protease, a threonine protease, a cysteine protease, an aspartate protease, a glutamic acid protease, or a metalloprotease. In some preferred embodiments the protease is a protease found in an Antigen Presenting Cell (APC). Thus, protease cleavage sites correspond to high abundance (highly expressed) proteases in APCs. A cleavage sensitive site that is sensitive to an APC enzyme is referred to as an APC cleavage sensitive site. Proteases expressed in APCs include but are not limited to Cysteine proteases, such as: Cathepsin B, Cathepsin H, Cathepsin L, Cathepsin S, Cathepsin F, Cathepsin Z, Cathepsin V, Cathepsin O, Cathepsin C, and Cathepsin K, and Aspartic proteases such as Cathepsin D, Cathepsin E, and Asparaginyl endopeptidase.

The cleavage sensitive site may preferably be a cathepsin B or S sensitive site. Exemplary cathepsin B sensitive sites include but are not limited to those described in WO2017/020026 (which is herein incorporated by reference) (see SEQ ID NOs: 12 to 407 of WO2017/020026). Exemplary cathepsin S sensitive sites include but are not limited to those described in WO2017/020026 (see SEQ ID NOs: 3 to 5, 408 to 1122 of WO2017/020026). Other cathepsin sensitive sites are known in the art or can easily be determined experimentally using digestion assays with no more than routine experimentation.

### Modifications of Nucleotides

The mRNA cancer vaccines of the present invention comprise one or more polynucleotides, which encode one or more immunogenic peptide fragment sequences. Exemplary polynucleotides may include at least one chemical modification. The polynucleotides can include various substitutions and/or insertions. As used herein in a polynucleotide, the terms “chemical modification” or, as appropriate, “chemically modified” refer to modification with respect to adenosine (A), guanosine (G), uridine (U), thymidine (T) or cytidine (C) ribo- or deoxyribnucleosides in one or more of their position, pattern, percent or population.

A modified polynucleotide may, when introduced to a cell or organism, exhibit reduced degradation in the cell or organism, as compared to an unmodified polynucleotide. A modified polynucleotide may, when introduced into a call or organism, exhibit reduced immunogenicity in the cell or organism (e.g., a reduced innate response.). Modifications of polynucleotides are well known in the art and include, for example, those listed in WO2017/020026 (which is herein incorporated by reference). Generally, the modifications discussed in this section are not intended to refer to the ribonucleotide modifications in naturally occurring 5′-terminal mRNA cap moieties.

The polynucleotide may comprise modifications which are naturally occurring, non-naturally occurring or the polynucleotide can comprise both naturally and non-naturally occurring modifications. The polynucleotides of the mRNA cancer vaccines of the invention can include any useful modification, such as to the sugar, the nucleobase, or the internucleoside linkage (e.g. to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications according to the present invention may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein. Non-natural modified nucleotides may be introduced to polynucleotides, e.g., of the mRNA cancer vaccines, or nucleic acids during synthesis or post-synthesis of the chains to achieve desired functions or properties. The modifications may be on internucleotide lineage, the purine or pyrimidine bases, or sugar. The modification may be introduced at the terminal of a chain or anywhere else in the chain; with chemical synthesis or with a polymerase enzyme. Any of the regions of the polynucleotides may be chemically modified.

The present disclosure provides for modified nucleosides and nucleotides. As described herein “nucleoside” is defined as a compound containing a sugar molecule (e.g., a pentose or ribose) or a derivative thereof in combination with an organic base (e.g., a purine or pyrimidine) or a derivative thereof (also referred to herein as “nucleobase”). As described herein, “nucleotide” is defined as a nucleoside including a phosphate group. The modified nucleotides may by synthesized by any useful method, as described herein (e.g., chemically, enzymatically, or recombinantly to include one or more modified or non-natural nucleosides). The polynucleotides may comprise a region or regions of linked nucleosides. Such regions may have variable backbone linkages. The linkages may be standard phosphodiester linkages, in which case the polynucleotides would comprise regions of nucleotides.

The modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil. Any combination of base/sugar or linker may be incorporated into the polynucleotides of the invention.

The mRNA of the invention may at least one chemical modification, which is preferably selected from pseudouridine, Nl-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-seudouridine, 5-aza-uridine, dihydropseudouridine, 5-methyluridine, 5-methoxyuridine, and 2′-O-methyl uridine.

### 5′ Caps and 3′ Tails

The present invention involves a messenger RNA (mRNA). As used herein, “messenger RNA” (mRNA) refers to any polynucleotide which encodes at least one peptide or polypeptide of interest and which is capable of being translated to produce the encoded peptide polypeptide of interest in vitro, in vivo, in situ or ex vivo. The basic components of an mRNA molecule include at least a coding region, a 5′UTR, a 3′UTR, a 5′ terminal cap and a 3′ tailing sequence. The mRNA of the invention typically includes all of these features.

A “5′ untranslated region (UTR)” is a region of an mRNA that is directly upstream (i.e., 5′) from the start codon (i.e., the first codon of an mRNA transcript translated by a ribosome) that does not encode a protein or peptide.

A “3′ untranslated region (UTR)” is a region of an mRNA that is directly downstream (i.e., 3′) from the stop codon (i.e., the codon of an mRNA transcript that signals a termination of translation) that does not encode a protein or peptide.

An “open reading frame” is a continuous stretch of DNA beginning with a start codon (e.g., methionine (ATG)), and ending with a stop codon (e.g., TAA, TAG or TGA) and encodes a protein or peptide.

A 5′ terminal cap is a specially altered nucleotide on the 5′ end of some primary transcripts such as messenger RNA, which promotes stability and translation. It usually consists of a guanine nucleotide connected to mRNA via an unusual 5′ to 5′ triphosphate linkage. This guanosine is methylated on the 7 position directly after capping in vivo by a methyltransferase. It may thus be referred to as a 7-methylguanylate cap, abbreviated as m7G. A preferred 5′ terminal cap is m7G(5′)ppp(5′)NlmpNp.

The 3′ tailing sequence is a polyA tail, a polyA-G quartet and/or a stem loop sequence. The 3′ tailing sequence is typically between 40 and 200 nucleotides in length. In some embodiments, the 3′ tailing sequence is a polyA tail. A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo, etc.) the poly(A) tail functions to protect mRNA from enzymatic degradation, e.g., in the cytoplasm, and aids in transcription termination, export of the mRNA from the nucleus, and translation.

In some embodiments, the polynucleotide includes from about 200 to about 3,000 nucleotides (e.g., from 200 to 500, from 200 to 1,000, from 200 to 1,500, from 200 to 3,000, from 500 to 1,000, from 500 to 1,500, from 500 to 2,000, from 500 to 3,000, from 1,000 to 1,500, from 1,000 to 2,000, from 1,000 to 3,000, from 1,500 to 3,000, and from 2,000 to 3,000).

The polynucleotides of the present invention may function as mRNA but are distinguished from wild-type mRNA in functional and/or structural features. mRNA cancer vaccines of the present invention may be encoded by in vitro translated (IVT) polynucleotides. An “in vitro transcription template (IVT),” as used herein, refers to deoxyribonucleic acid (DNA) suitable for use in an IVT reaction for the production of messenger RNA (mRNA). In some embodiments, an IVT template encodes a 5′ untranslated region, contains an open reading frame, and encodes a 3′ untranslated region and a polyA tail. The particular nucleotide sequence composition and length of an IVT template will depend on the mRNA of interest encoded by the template.

Methods of Preparing mRNA

Also disclosed are a method for preparing an mRNA cancer vaccine. mRNA may be prepared by any suitable technique known in the art, via any appropriate synthesis route. IVT methods are preferred. In vitro transcription (IVT) methods permit template-directed synthesis of RNA molecules of almost any sequence. The size of the RNA molecules that can be synthesized using IVT methods range from short oligonucleotides to long nucleic acid polymers of several thousand bases. IVT methods permit synthesis of large quantities of RNA transcript (e.g., from microgram to milligram quantities) (Beckert et ah, Synthesis of RNA by in vitro transcription, Methods Mol Biol. 703:29-41 (2011); Rio et al. RNA: A Laboratory Manual. Cold Spring Harbor: Cold Spring Harbor Laboratory Press, 2011, 205-220.; Cooper, Geoffery M. The Cell: A Molecular Approach. 4th ed. Washington D.C.: ASM Press, 2007. 262-299). Generally, IVT utilizes a DNA template featuring a promoter sequence upstream of a sequence of interest. The promoter sequence is most commonly of bacteriophage origin (ex. the T7, T3 or SP6 promoter sequence) but many other promotor sequences can be tolerated including those designed de novo. Transcription of the DNA template is typically best achieved by using the RNA polymerase corresponding to the specific bacteriophage promoter sequence. Exemplary RNA polymerases include, but are not limited to T7 RNA polymerase, T3 RNA polymerase, or SP6 RNA polymerase, among others. IVT is generally initiated at a dsDNA but can proceed on a single strand. Suitable methods include, for example, those listed in WO2017/020026 (which is herein incorporated by reference).

### Codon Optimisation

The mRNA disclosed herein may be in whole or in part codon optimized for human expression and/or for reducing immune recognition. Codon optimization methods are known in the art and may be useful in efforts to achieve various results, such as to match codon frequencies in target and host organisms to ensure proper folding, bias GC content to increase mRNA stability or reduce secondary structures, minimize tandem repeat codons or base runs that may impair gene construction or expression, customize transcriptional and translational control regions, insert or remove protein trafficking sequences, remove/add post translation modification sites in encoded protein (e.g. glycosylation sites), add, remove or shuffle protein domains, insert or delete restriction sites, modify ribosome binding sites and mRNA degradation sites, to adjust translational rates to allow the various domains of the protein to fold properly, or to reduce or eliminate problem secondary structures within the polynucleotide. Codon optimization tools, algorithms and services are known in the art, non-limiting examples include services from GeneArt (Life Technologies), DNA2.0 (Menlo Park CA) and/or proprietary methods. Preferably, the ORF sequence is optimized using optimization algorithms.

A codon optimized sequence may share less than 95%, 90%, 85%, 80%, or 75% sequence identity to a naturally-occurring or wild-type sequence (e.g., a naturally-occurring or wild-type mRNA sequence encoding a polypeptide or protein of interest. A codon optimized sequence may share between 65% and 85% sequence identity to a naturally-occurring or wild-type sequence (e.g., a naturally-occurring or wild-type mRNA sequence encoding a polypeptide or protein of interest.

An exemplary codon optimized RNA may be one in which the levels of G/C are enhanced. The G/C-content of nucleic acid molecules may influence the stability of the RNA. RNA having an increased amount of guanine (G) and/or cytosine (C) residues may be functionally more stable than nucleic acids containing a large amount of adenine (A) and thymine (T) or uracil (U) nucleotides. WO02/098443 (herein incorporated by reference) discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

### Method for the Prevention or Treatment of Cancer

Also disclosed is a method for preventing or treating cancer, comprising administering the mRNA described herein.

The cancer may be prostate cancer, brain cancer, breast cancer, colorectal cancer, pancreatic cancer, ovarian cancer, lung cancer, cervical cancer, liver cancer, head/neck/throat cancer, skin cancer, bladder cancer or a hematologic cancer. The cancer may take the form of a tumour or a blood born cancer. The tumour may be solid. The tumour is typically malignant and may be metastatic. The tumour may be an adenoma, an adenocarcinoma, a blastoma, a carcinoma, a desmoid tumour, a desmopolastic small round cell tumour, an endocrine tumour, a germ cell tumour, a lymphoma, a leukaemia, a sarcoma, a Wilms tumour, a lung tumour, a colon tumour, a lymph tumour, a breast tumour or a melanoma.

Types of blastoma include hepatblastoma, glioblastoma, neuroblastoma or retinoblastoma. Types of carcinoma include colorectal carcinoma or heptacellular carcinoma, pancreatic, prostate, gastric, esophegal, cervical, and head and neck carcinomas, and adenocarcinoma. Types of sarcoma include Ewing sarcoma, osteosarcoma, rhabdomyosarcoma, or any other soft tissue sarcoma. Types of melanoma include Lentigo maligna, Lentigo maligna melanoma, Superficial spreading melanoma, Acral lentiginous melanoma, Mucosal melanoma, Nodular melanoma, Polypoid melanoma, Desmoplastic melanoma, Amelanotic melanoma, Soft-tissue melanoma, Melanoma with small nevus-like cells, Melanoma with features of a Spitz nevus and Uveal melanoma. Types of lymphoma and leukaemia include Precursor T-cell leukemia/lymphoma, acute myeloid leukaemia, chronic myeloid leukaemia, acute lymphcytic leukaemia, Follicular lymphoma, Diffuse large B cell lymphoma, Mantle cell lymphoma, chronic lymphocytic leukemia/lymphoma, MALT lymphoma, Burkitt's lymphoma, Mycosis fungoides, Peripheral T-cell lymphoma, Nodular sclerosis form of Hodgkin lymphoma, Mixed-cellularity subtype of Hodgkin lymphoma. Types of lung tumour include tumours of non-small-cell lung cancer (adenocarcinoma, squamous-cell carcinoma and large-cell carcinoma) and small-cell lung carcinoma.

The method of the invention works by activating or augmenting the T cell anti-cancer response in a subject. This is achieved by increasing cancer or tumour-specific effector T cell activation, by blocking or inhibiting one or more immune checkpoints. The method of the invention blocks or inhibits said one or more immune checkpoints by administering the mRNA, which results in expression of immunogenic peptide fragments of a component of the checkpoint, thereby eliciting an immune response in the subject against said component, and thereby blocking or inhibiting the activity of the checkpoint. Thus it may alternatively be described as a vaccine against the said component of the said checkpoint. The component of the checkpoint which is targeted by the said immune response is preferably expressed by tumour cells and may also be expressed by normal cells which have an immune inhibitory effect. Accordingly, the said immune response has a double effect in that it both blocks and inhibits the activity of the checkpoint and also directly attacks the tumour.

The mRNA may advantageously encode immunogenic peptide fragments of more than one checkpoint component, from the same or different checkpoints. By blocking or inhibiting multiple immune system checkpoints, the method of the invention will result in a greater anti-tumour response with fewer side-effects or complications as compared to alternative methods. The anti-tumour response is typically greater than that which would be expected if only a single checkpoint were targeted. In addition, there are less likely to be reductions in efficacy due to anti-drug responses, since the first approach (the vaccine) will actively benefit from such a response, which may also result in a long lasting effect. These benefits apply even if both approaches target the same immune system checkpoint.

The mRNA or mRNA cancer vaccine may be administered by any route, for example intranasally, intravenously (IV), intradermally, intramuscularly (IM), or intraperitoneally. In some embodiments, the administration is a single administration. IV and IM are preferred. In some embodiments, the subject is administered the vaccine more than once.

The mRNA cancer vaccine may be used as a therapeutic or a prophylactic. The effective amount of the polynucleotides of the mRNA cancer vaccines of the invention provided to a cell, a tissue or a subject may be enough for immune activation, and in particular antigen specific immune activation. A prophylactically effective dose may be a therapeutically effective dose that prevents advancement of cancer at a clinically acceptable level. The administration may be a single administration or the subject may be administered the vaccine more than once.

Provided are compositions and methods for treatment or prevention of a disease or condition in humans and other mammals. The active therapeutic agents include the mRNA cancer vaccines, cells containing mRNA cancer vaccines or polypeptides translated from the polynucleotides contained in the mRNA cancer vaccines.

The mRNA cancer vaccines may be induced for translation of a polypeptide (e.g., antigen or immunogen) in a cell, tissue or organism. Such translation can be in vivo, ex vivo, in culture, or in vitro. The cell, tissue or organism is contacted with an effective amount of a composition containing a mRNA cancer vaccine which contains a polynucleotide that has at least one a translatable region encoding immunogenic peptide fragments as described herein. An “effective amount” of the mRNA cancer vaccine is provided based, at least in part, on the target tissue, target cell type, means of administration, physical characteristics of the polynucleotide (e.g., size, and extent of modified nucleosides) and other components of the mRNA cancer vaccine, and other determinants. In general, an effective amount of the mRNA cancer vaccine composition provides an induced or boosted immune response as a function of production of the encoded immunogenic peptide fragment(s) in the cell, preferably more efficient than a composition containing a corresponding unmodified polynucleotide encoding the same antigen or a peptide antigen. Increased production may be demonstrated by increased cell transfection (i.e., the percentage of cells transfected with the mRNA cancer vaccine), increased protein translation from the polynucleotide, decreased nucleic acid degradation (as demonstrated, e.g., by increased duration of protein translation from a modified polynucleotide), or altered antigen specific immune response of the host cell.

The method may also comprises the administration of one or more other therapeutic agents. The other agent may preferably be an “immunomodulatory agent”, which as used herein means any agent which, when administered to a subject, blocks or inhibits the action of an immune system checkpoint, resulting in the upregulation of an immune effector response in the subject, typically a T cell effector response, which preferably comprises an anti-tumour T cell effector response. The immunomodulatory agent used in the method of the present invention may block or inhibit any of the immune system checkpoints described above. The agent may be an antibody or any other suitable agent which results in said blocking or inhibition. The agent may thus be referred to generally as an inhibitor of a said checkpoint.

An “antibody” as used herein includes whole antibodies and any antigen binding fragment (i.e., “antigen-binding portion”) or single chains thereof. An antibody may be a polyclonal antibody or a monoclonal antibody and may be produced by any suitable method. Examples of binding fragments encompassed within the term “antigen-binding portion” of an antibody include a Fab fragment, a F(ab′)2 fragment, a Fab′ fragment, a Fd fragment, a Fv fragment, a dAb fragment and an isolated complementarity determining region (CDR). Single chain antibodies such as scFv and heavy chain antibodies such as VHH and camel antibodies are also intended to be encompassed within the term “antigen-binding portion” of an antibody.

Preferred antibodies which block or inhibit the CTLA-4 interaction with B7 proteins include ipilumumab, tremelimumab, or any of the antibodies disclosed in WO2014/207063. Other molecules include polypeptides, or soluble mutant CD86 polypeptides.

Preferred antibodies which block or inhibit the PD1 interaction with PD-L1 include Nivolumab, Pembrolizumab, Lambrolizumab, Pidilzumab, and AMP-224. Anti-PD-L1 antibodies include MEDI-4736 and MPDL3280A. Pembrolizumab and Nivolumab are particularly preferred.

Other suitable inhibitors include small molecule inhibitors (SMI), which are typically small organic molecules. Preferred inhibitors of IDO1 include Epacadostat (INCB24360), Indoximod, GDC-0919 (NLG919) and F001287. Other inhibitors of IDO1 include 1-methyltryptophan (1MT).

An immunodmodulatory agent, such as an antibody or SMI, may be formulated with a pharmaceutically acceptable excipient or other auxiliary substance for administration to a subject. Suitable excipients and auxiliary substances are known in the art. Suitable forms for preparation, packaging and sale of the immunotherapeutic composition are also known in the art.

The mRNA cancer vaccine and other therapeutic agent may be administered simultaneously or sequentially. When the other therapeutic agents are administered simultaneously they can be administered in the same or separate formulations, but are administered at the same time. The other therapeutic agents are administered sequentially with one another and with the mRNA cancer vaccine, when the administration of the other therapeutic agents and the mRNA cancer vaccine is temporally separated. The separation in time between the administration of these compounds may be a matter of minutes or it may be longer, e.g. hours, days, weeks, months.

The present invention provides methods comprising administering mRNA cancer vaccines and in accordance with the invention to a subject in need thereof. The exact amount required will vary from subject to subject, depending on the species, age, and general condition of the subject, the severity of the disease, the particular composition, its mode of administration, its mode of activity, and the like. Compositions in accordance with the invention are typically formulated in dosage unit form for ease of administration and uniformity of dosage. It will be understood, however, that the total daily usage of the compositions of the present invention may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the art.

Compositions in accordance with the present invention may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect (see e.g., the range of unit doses described in International Publication No WO2013078199, herein incorporated by reference in its entirety).

The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. A mRNA cancer vaccine pharmaceutical composition described herein can be formulated into a dosage form described herein, such as an intranasal, intratracheal, or injectable (e.g., intravenous, intraocular, intravitreal, intramuscular, intradermal, intracardiac, intraperitoneal, and subcutaneous).

The RNA (e.g., mRNA) vaccine compositions may be administered at dosage levels sufficient to deliver 0.0001 mg/kg to 100 mg/kg, 0.001 mg/kg to 0.05 mg/kg, 0.005 mg/kg to 0.05 mg/kg, 0.001 mg/kg to 0.005 mg/kg, 0.05 mg/kg to 0.5 mg/kg, 0.01 mg/kg to 50 mg/kg, 0.1 mg/kg to 40 mg/kg, 0.5 mg/kg to 30 mg/kg, 0.01 mg/kg to 10 mg/kg, 0.1 mg/kg to 10 mg/kg, or 1 mg/kg to 25 mg/kg, of subject body weight per day, one or more times a day, per week, per month, etc. to obtain the desired therapeutic, diagnostic, prophylactic, or imaging effect (see e.g., the range of unit doses described in International Publication No WO2013078199, herein incorporated by reference in its entirety). The desired dosage may be delivered three times a day, two times a day, once a day, every other day, every third day, every week, every two weeks, every three weeks, every four weeks, every 2 months, every three months, every 6 months, etc. In certain embodiments, the desired dosage may be delivered using multiple administrations (e.g., two, three, four, five, six, seven, eight, nine, ten, eleven, twelve, thirteen, fourteen, or more administrations).

When multiple administrations are employed, split dosing regimens such as those described herein may be used. In some embodiments, the RNA vaccine compositions may be administered at dosage levels sufficient to deliver 0.0005 mg/kg to 0.01 mg/kg, e.g., about 0.0005 mg/kg to about 0.0075 mg/kg, e.g., about 0.0005 mg/kg, about 0.001 mg/kg, about 0.002 mg/kg, about 0.003 mg/kg, about 0.004 mg/kg or about 0.005 mg/kg. In some embodiments, the RNA vaccine compositions may be administered once or twice (or more) at dosage levels sufficient to deliver 0.025 mg/kg to 0.250 mg/kg, 0.025 mg/kg to 0.500 mg/kg, 0.025 mg/kg to 0.750 mg/kg, or 0.025 mg/kg to 1.0 mg/kg.

RNA vaccine compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.0100 mg, 0.025 mg, 0.050 mg, 0.075 mg, 0.100 mg, 0.125 mg, 0.150 mg, 0.175 mg, 0.200 mg, 0.225 mg, 0.250 mg, 0.275 mg, 0.300 mg, 0.325 mg, 0.350 mg, 0.375 mg, 0.400 mg, 0.425 mg, 0.450 mg, 0.475 mg, 0.500 mg, 0.525 mg, 0.550 mg, 0.575 mg, 0.600 mg, 0.625 mg, 0.650 mg, 0.675 mg, 0.700 mg, 0.725 mg, 0.750 mg, 0.775 mg, 0.800 mg, 0.825 mg, 0.850 mg, 0.875 mg, 0.900 mg, 0.925 mg, 0.950 mg, 0.975 mg, or 1.0 mg. Higher and lower dosages and frequency of administration are encompassed by the present disclosure. For example, a the RNA vaccine composition may be administered three or four times.

RNA vaccine compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.010 mg, 0.025 mg, 0.100 mg or 0.400 mg.

An mRNA vaccine for use in a method of vaccinating a subject may be administered to the subject as a single dosage of between 10 μg/kg and 400 μg/kg of the nucleic acid vaccine in an effective amount to vaccinate the subject. The RNA vaccine for use in a method of vaccinating a subject may be administered to the subject a single dosage of between 10 μg and 400 μg of the nucleic acid vaccine in an effective amount to vaccinate the subject.

### Compositions, Formulations, Encapsulations

Also disclosed are pharmaceutical compositions including mRNA cancer vaccines and mRNA cancer vaccine compositions and/or complexes optionally in combination with one or more pharmaceutically acceptable excipients. The vaccines may be formulated or administered alone or in conjunction with one or more other components. For instance the vaccine compositions of the invention may comprise other components including, but not limited to, adjuvants. Optionally the vaccine is free of adjuvants.

Pharmaceutical compositions may optionally comprise one or more additional active substances, e.g. therapeutically and/or prophylactically active substances. Pharmaceutical compositions of the present invention may be sterile and/or pyrogen-free. General considerations in the formulation and/or manufacture of pharmaceutical agents may be found, for example, in Remington: The Science and Practice of Pharmacy 21st ed., Lippincott Williams & Wilkins, 2005 (incorporated herein by reference in its entirety). Compositions may typically be administered to humans, human patients or subjects. For the purposes of the present disclosure, the phrase “active ingredient” generally refers to the mRNA cancer vaccines or the polynucleotides contained therein, e.g., polynucleotides, for example, mRNAs, encoding immunogenic peptide fragments, to be delivered as described herein.

Formulations of the pharmaceutical compositions described herein may be prepared by any method known or hereafter developed in the art. In general, such preparatory methods include the step of bringing the active ingredient into association with an excipient and/or one or more other accessory ingredients, and then, if necessary and/or desirable, dividing, shaping and/or packaging the product into a desired single- or multi-dose unit. Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

mRNA cancer vaccines may be formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles, peptides, proteins, cells transfected with mRNA cancer vaccines (e.g., for transplantation into a subject), hyaluronidase, nanoparticle mimics and combinations thereof.

The mRNA and/or compositions disclosed herein may include stabilising elements. Naturally-occurring eukaryotic mRNA molecules have been found to contain stabilizing elements, including, but not limited to the 5′ and 3′ UTRs, the 5′ cap and the 3′ tail discussed elsewhere in this document. Other stabilizing elements that may be included in mRNA as disclosed herein may include for instance a histone stem-loop. In some embodiments, the histone stem-loop is generally derived from histone genes, and includes an intramolecular base pairing of two neighbored partially or entirely reverse complementary sequences separated by a spacer, consisting of a short sequence, which forms the loop of the structure. The mRNA may have one or more AU-rich sequences removed. Such sequences may be destabilising. The RNA vaccine may or may not contain a enhancer and/or promoter sequence, which may be modified or unmodified or which may be activated or inactivated.

In preferred embodiments, the mRNA cancer vaccine may be formulated in a lipid-polycation complex. The formation of the lipid-polycation complex may be accomplished by methods known in the art and/or as described in U.S. Pub. No. 20120178702, herein incorporated by reference in its entirety. As a non-limiting example, the polycation may include a cationic peptide or a polypeptide such as, but not limited to, polylysine, poly ornithine and/or polyarginine and the cationic peptides described in International Pub. No. WO2012013326 or US Patent Pub. No. US20130142818; each of which is herein incorporated by reference in its entirety. In another embodiment, the mRNA cancer vaccines may be formulated in a lipid-polycation complex which may further include a non-cationic lipid such as, but not limited to, cholesterol or dioleoyl-phosphatidylethanolamine (DOPE). The liposome formulation may be influenced by, but not limited to, the selection of the cationic lipid component, the degree of cationic lipid saturation, the nature of the PEGylation, ratio of all components and biophysical parameters such as size. In one example by Semple et al. (Semple et al. Nature Biotech. 2010 28:172-176; herein incorporated by reference in its entirety), the liposome formulation was composed of 57.1% cationic lipid, 7.1% dipalmitoylphosphatidylcholine, 34.3% cholesterol, and 1.4% PEG-c-DMA. As another example, changing the composition of the cationic lipid could more effectively deliver siRNA to various antigen presenting cells (Basha et al. Mol Ther. 2011 19:2186-2200; herein incorporated by reference in its entirety).

Liposome formulations may comprise from about 35 to about 45% cationic lipid, from about 40% to about 50% cationic lipid, from about 50% to about 60% cationic lipid and/or from about 55% to about 65% cationic lipid. In some embodiments, the ratio of lipid to mRNA in liposomes may be from about 5:1 to about 20:1, from about 10:1 to about 25:1, from about 15:1 to about 30:1 and/or at least 30:1. The ratio of PEG in the lipid nanoparticle (LNP) formulations may be increased or decreased and/or the carbon chain length of the PEG lipid may be modified from C 14 to C18 to alter the pharmacokinetics and/or biodistribution of the LNP formulations. As a non-limiting example, LNP formulations may contain from about 0.5% to about 3.0%, from about 1.0% to about 3.5%, from about 1.5% to about 4.0%, from about 2.0% to about 4.5%, from about 2.5% to about 5.0% and/or from about 3.0% to about 6.0% of the lipid molar ratio of PEG-c-DOMG (R-3-[(co-methoxy-poly(ethyleneglycol) 2000) carbamoyl)]-1,2-dimyristyloxypropyl-3-amine) (also referred to herein as PEG-DOMG) as compared to the cationic lipid, DSPC and cholesterol. In another embodiment the PEG-c-DOMG may be replaced with a PEG lipid such as, but not limited to, PEG-DSG(1,2-Distearoyl-sn-glycerol, methoxypolyethylene glycol), PEG-DMG(1,2-Dimyristoyl-sn-glycerol) and/or PEG-DPG(1,2-Dipalmitoyl-sn-glycerol, methoxypolyethylene glycol). The cationic lipid may be selected from any lipid known in the art such as, but not limited to, DLin-MC3-DMA, D Lin-DMA, C 12-200 and DLin-KC2-DMA.

The mRNA cancer vaccine formulation comprising the polynucleotide may be a nanoparticle which may comprise at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In another aspect, the lipid may be a cationic lipid such as, but not limited to, DLin-DMA, DLin-D-DMA, DLin-MC3-DMA, DLin-KC2-DMA, DODMA and amino alcohol lipids. The amino alcohol cationic lipid may be the lipids described in and/or made by the methods described in US Patent Publication No. US20130150625, herein incorporated by reference in its entirety. As a non-limiting example, the cationic lipid may be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in US20130150625); or any pharmaceutically acceptable salt or stereoisomer thereof.

Lipid nanoparticle formulations typically comprise a lipid, in particular, an ionizable cationic lipid, for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), and further comprise a neutral lipid, a sterol and a molecule capable of reducing particle aggregation, for example a PEG or PEG-modified lipid. The lipid nanoparticle formulation may consist essentially of (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319); (ii) a neutral lipid selected from DSPC, DPPC, POPC, DOPE and SM; (iii) a sterol, e.g., cholesterol; and (iv) a PEG-lipid, e.g., PEG-DMG or PEG-cDMA, in a molar ratio of about 20-60% cationic lipid:5-25% neutral lipid:25-55% sterol; 0.5-15% PEG-lipid. The formulation may include from about 25% to about 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., from about 35 to about 65%, from about 45 to about 65%, about 60%, about 57.5%, about 50% or about 40% on a molar basis.

The formulation may include from about 0.5% to about 15% on a molar basis of the neutral lipid e.g., from about 3 to about 12%, from about 5 to about 10% or about 15%, about 10%, or about 7.5% on a molar basis. Exemplary neutral lipids include, but are not limited to, DSPC, POPC, DPPC, DOPE and SM. The formulation may include from about 5% to about 50% on a molar basis of the sterol (e.g., about 15 to about 45%, about 20 to about 40%, about 40%, about 38.5%, about 35%, or about 31% on a molar basis. An exemplary sterol is cholesterol. The formulation may include from about 0.5% to about 20% on a molar basis of the PEG or PEG-modified lipid (e.g., about 0.5 to about 10%, about 0.5 to about 5%, about 1.5%, about 0.5%, about 1.5%, about 3.5%, or about 5% on a molar basis. The PEG or PEG modified lipid may comprise a PEG molecule of an average molecular weight of 2,000 Da, or an average molecular weight of less than 2,000, for example around 1,500 Da, around 1,000 Da, or around 500 Da. Exemplary PEG-modified lipids include, but are not limited to, PEG-distearoyl glycerol (PEG-DMG) (also referred herein as PEG-C14 or C14-PEG), PEG-CDMA (further discussed in Reyes et al. J. Controlled Release, 107, 276-287 (2005) the contents of which are herein incorporated by reference in its entirety)

The formulations herein may include 25-75% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 0.5-15% of the neutral lipid, 5-50% of the sterol, and 0.5-20% of the PEG or PEG-modified lipid on a molar basis. In one embodiment, the formulations of the inventions include 35-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 3-12% of the neutral lipid, 15-45% of the sterol, and 0.5-10% of the PEG or PEG-modified lipid on a molar basis. The formulations herein may include 45-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 5-10% of the neutral lipid, 25-40% of the sterol, and 0.5-10% of the PEG or PEG-modified lipid on a molar basis. The formulations herein may include about 60% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 7.5% of the neutral lipid, about 31% of the sterol, and about 1.5% of the PEG or PEG-modified lipid on a molar basis.

The formulations herein may include about 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 10% of the neutral lipid, about 38.5% of the sterol, and about 1.5% of the PEG or PEG-modified lipid on a molar basis.

The formulations herein may include include about 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 10% of the neutral lipid, about 35% of the sterol, about 4.5% or about 5% of the PEG or PEG-modified lipid, and about 0.5% of the targeting lipid on a molar basis.

The formulations herein may include about 40% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 15% of the neutral lipid, about 40% of the sterol, and about 5% of the PEG or PEG-modified lipid on a molar basis. In one embodiment, the formulations include about 57.2% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 7.1% of the neutral lipid, about 34.3% of the sterol, and about 1.4% of the PEG or PEG-modified lipid on a molar basis.

The formulations herein may include about 57.5% of a cationic lipid selected from the PEG lipid is PEG-CDMA (PEG-CDMA is further discussed in Reyes et al. (J. Controlled Release, 107, 276-287 (2005), the contents of which are herein incorporated by reference in its entirety), about 7.5% of the neutral lipid, about 31.5% of the sterol, and about 3.5% of the PEG or PEG-modified lipid on a molar basis.

In preferred embodiments, lipid nanoparticle formulation consists essentially of a lipid mixture in molar ratios of about 20-70% cationic lipid:5-45% neutral lipid:20-55% cholesterol: 0.5-15% PEG-modified lipid; more preferably in a molar ratio of about 20-60% cationic lipid:5-25% neutral lipid:25-55% cholesterol: 0.5-15% PEG-modified lipid. In particular embodiments, the molar lipid ratio is approximately 50/10/38.5/1.5 (mol % cationic lipid/neutral lipid, e.g., DSPC/Chol/PEG-modified lipid, e.g., PEG-DMG, PEG-DSG or PEG-DPG), 57.2/7.1134.3/1.4 (mol % cationic lipid/neutral lipid, e.g., DPPC/Chol/PEG-modified lipid, e.g., PEG-cDMA), 40/15/40/5 (mol % cationic lipid/neutral lipid, e.g., DSPC/Chol/PEG-modified lipid, e.g., PEG-DMG), 50/10/35/4.5/0.5 (mol % cationic lipid/neutral lipid, e.g., DSPC/Chol/PEG-modified lipid, e.g., PEG-DSG), 50/10/35/5 (cationic lipid/neutral lipid, e.g., DSPC/Chol/PEG-modified lipid, e.g., PEG-DMG), 40/10/40/10 (mol % cationic lipid/neutral lipid, e.g., DSPC/Chol/PEG-modified lipid, e.g., PEG-DMG or PEG-cDMA), 35/15/40/10 (mol % cationic lipid/neutral lipid, e.g., DSPC/Chol/PEG-modified lipid, e.g., PEG-DMG or PEG-cDMA) or 52/13/30/5 (mol % cationic lipid/neutral lipid, e.g., DSPC/Chol/PEG-modified lipid, e.g., PEG-DMG or PEG-cDMA).

Exemplary lipid nanoparticle compositions and methods of making same are described, for example, in Semple et al. (2010) Nat. Biotechnol. 28:172-176; Jayarama et al. (2012), Angew. Chem. Int. Ed., 51:8529-8533; and Maier et al. (2013) Molecular Therapy 21, 1570-1578 (the contents of each of which are incorporated herein by reference in their entirety).

The lipid nanoparticle formulations described herein may comprise a cationic lipid, a PEG lipid and a structural lipid and optionally comprise a non-cationic lipid. As a non-limiting example, the lipid nanoparticle may comprise about 40-60% of cationic lipid, about 5-15% of a non-cationic lipid, about 1-2% of a PEG lipid and about 30-50% of a structural lipid. As another non-limiting example, the lipid nanoparticle may comprise about 50% cationic lipid, about 10% non-cationic lipid, about 1.5% PEG lipid and about 38.5% structural lipid. As yet another non-limiting example, the lipid nanoparticle may comprise about 55% cationic lipid, about 10% non-cationic lipid, about 2.5% PEG lipid and about 32.5% structural lipid. In one embodiment, the cationic lipid may be any cationic lipid described herein such as, but not limited to, DLin-KC2-DMA, DLin-MC3-DMA and L319.

The lipid nanoparticle formulations described herein may be 4 component lipid nanoparticles. The lipid nanoparticle may comprise a cationic lipid, a non-cationic lipid, a PEG lipid and a structural lipid. As a non-limiting example, the lipid nanoparticle may comprise about 40-60% of cationic lipid, about 5-15% of a non-cationic lipid, about 1-2% of a PEG lipid and about 30-50% of a structural lipid. As another non-limiting example, the lipid nanoparticle may comprise about 50% cationic lipid, about 10% non-cationic lipid, about 1.5% PEG lipid and about 38.5% structural lipid. As yet another non-limiting example, the lipid nanoparticle may comprise about 55% cationic lipid, about 10% non-cationic lipid, about 2.5% PEG lipid and about 32.5% structural lipid. In one embodiment, the cationic lipid may be any cationic lipid described herein such as, but not limited to, DLin-KC2-DMA, DLin-MC3-DMA and L319.

The lipid nanoparticle formulations described herein may comprise a cationic lipid, a non-cationic lipid, a PEG lipid and a structural lipid. As a non-limiting example, the lipid nanoparticle comprise about 50% of the cationic lipid DLin-KC2-DMA, about 10% of the non-cationic lipid DSPC, about 1.5% of the PEG lipid PEG-DOMG and about 38.5% of the structural lipid cholesterol. As a non-limiting example, the lipid nanoparticle comprise about 50% of the cationic lipid DLin-MC3-DMA, about 10% of the non-cationic lipid DSPC, about 1.5% of the PEG lipid PEG-DOMG and about 38.5% of the structural lipid cholesterol. As a non-limiting example, the lipid nanoparticle comprise about 50% of the cationic lipid DLin-MC3-DMA, about 10% of the non-cationic lipid DSPC, about 1.5% of the PEG lipid PEG-DMG and about 38.5% of the structural lipid cholesterol. As yet another non-limiting example, the lipid nanoparticle comprise about 55% of the cationic lipid L319, about 10% of the non-cationic lipid DSPC, about 2.5% of the PEG lipid PEG-DMG and about 32.5% of the structural lipid cholesterol.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

The mRNA cancer vaccine composition may comprise the polynucleotide described herein, formulated in a lipid nanoparticle comprising MC3, Cholesterol, DSPC and PEG2000-DMG, the buffer trisodium citrate, sucrose and water for injection. As a non-limiting example, the composition comprises: 2.0 mg/mL of drug substance (e.g., polynucleotides encoding H10N8 influenza virus), 21.8 mg/mL of MC3, 10.1 mg/mL of cholesterol, 5.4 mg/mL of DSPC, 2.7 mg/mL of PEG2000-DMG, 5.16 mg/mL of trisodium citrate, 71 mg/mL of sucrose and about 1.0 mL of water for injection.

RNA vaccines can be formulated using one or more liposomes, lipoplexes, or lipid nanoparticles. Pharmaceutical compositions of RNA vaccines may include liposomes. Liposomes are artificially-prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations. The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

As a non-limiting example, liposomes such as synthetic membrane vesicles may be prepared by the methods, apparatus and devices described in US Patent Publication No. US20130177638, US20130177637, US20130177636, US20130177635, US20130177634, US20130177633, US20130183375, US20130183373 and US20130183372, the contents of each of which are herein incorporated by reference in its entirety. Pharmaceutical compositions described herein may include, without limitation, liposomes such as those formed from 1,2-dioleyloxy-N,N-dimethylaminopropane (DODMA) liposomes, DiLa2 liposomes from Marina Biotech (Bothell, WA), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety) and liposomes which may deliver small molecule drugs such as, but not limited to, DOXIL® from Janssen Biotech, Inc. (Horsham, PA).

Pharmaceutical compositions described herein may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; U.S. Patent Publication No US20130122104; all of which are incorporated herein in their entireties). The original manufacture method by Wheeler et al. was a detergent dialysis method, which was later improved by Jeffs et al. and is referred to as the spontaneous vesicle formation method. The liposome formulations are composed of 3 to 4 lipid components in addition to the polynucleotide.

As an example a liposome can contain, but is not limited to, 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al. As another example, certain liposome formulations may contain, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al.

Liposome formulations may comprise from about about 25.0% cholesterol to about 40.0% cholesterol, from about 30.0% cholesterol to about 45.0% cholesterol, from about 35.0% cholesterol to about 50.0% cholesterol and/or from about 48.5% cholesterol to about 60% cholesterol. In a preferred embodiment, formulations may comprise a percentage of cholesterol selected from the group consisting of 28.5%, 31.5%, 33.5%, 36.5%, 37.0%, 38.5%, 39.0% and 43.5%. In some embodiments, formulations may comprise from about 5.0% to about 10.0% DSPC and/or from about 7.0% to about 15.0% DSPC.

Pharmaceutical compositions may include liposomes which may be formed to deliver polynucleotides which may encode at least one immunogen (antigen) or any other polypeptide of interest. The RNA vaccine may be encapsulated by the liposome and/or it may be contained in an aqueous core which may then be encapsulated by the liposome (see International Pub. Nos. WO2012031046, WO2012031043, WO2012030901 and WO2012006378 and US Patent Publication No. US20130189351, US20130195969 and US20130202684; the contents of each of which are herein incorporated by reference in their entirety).

Liposomes may be formulated for targeted delivery. As a non-limiting example, the liposome may be formulated for targeted delivery to the liver. The liposome used for targeted delivery may include, but is not limited to, the liposomes described in and methods of making liposomes described in US Patent Publication No. US20130195967, the contents of which are herein incorporated by reference in its entirety. In another embodiment, the polynucleotide which may encode an immunogen (antigen) may be formulated in a cationic oil-in-water emulsion where the emulsion particle comprises an oil core and a cationic lipid which can interact with the polynucleotide anchoring the molecule to the emulsion particle (see International Pub. No. WO2012006380; herein incorporated by reference in its entirety).

RNA vaccines may also be formulated in a water-in-oil emulsion comprising a continuous hydrophobic phase in which the hydrophilic phase is dispersed. As a non-limiting example, the emulsion may be made by the methods described in International Publication No. WO201087791, the contents of which are herein incorporated by reference in its entirety. The lipid formulation may include at least cationic lipid, a lipid which may enhance transfection and a least one lipid which contains a hydrophilic head group linked to a lipid moiety (International Pub. No. WO2011076807 and U.S. Pub. No. 20110200582; the contents of each of which is herein incorporated by reference in their entirety). In another embodiment, the polynucleotides encoding an immunogen may be formulated in a lipid vesicle which may have crosslinks between functionalized lipid bilayers (see U.S. Pub. No. 20120177724, the contents of which is herein incorporated by reference in its entirety).

Polynucleotides may be formulated in a lipsome as described in International Patent Publication No. WO2013086526, the contents of which is herein incorporated by reference in its entirety. The RNA vaccines may be encapsulated in a liposome using reverse pH gradients and/or optimized internal buffer compositions as described in International Patent Publication No. WO2013086526, the contents of which is herein incorporated by reference in its entirety.

RNA vaccine pharmaceutical compositions may be formulated in liposomes such as, but not limited to, DiLa2 liposomes (Marina Biotech, Bothell, WA), SMARTICLES® (Marina Biotech, Bothell, WA), neutral DOPC (1,2-dioleoyl-sn-glycero-3-phosphocholine) based liposomes (e.g., siRNA delivery for ovarian cancer (Landen et al. Cancer Biology & Therapy 2006 5(12) 1708-1713); herein incorporated by reference in its entirety) and hyaluronan-coated liposomes (Quiet Therapeutics, Israel).

The cationic lipid may be a low molecular weight cationic lipid such as those described in U.S. patent application No. 20130090372, the contents of which are herein incorporated by reference in its entirety.

The RNA vaccines may be formulated in a lipid vesicle which may have crosslinks between functionalized lipid bilayers.

The RNA vaccines may be formulated in a liposome comprising a cationic lipid. The liposome may have a molar ratio of nitrogen atoms in the cationic lipid to the phophates in the RNA (N:P ratio) of between 1:1 and 20:1 as described in International Publication No. WO2013006825, herein incorporated by reference in its entirety. In another embodiment, the liposome may have a N:P ratio of greater than 20:1 or less than 1:1.

The RNA vaccines may be formulated in a lipid-polycation complex. The formation of the lipid-polycation complex may be accomplished by methods known in the art and/or as described in U.S. Pub. No. 20120178702, herein incorporated by reference in its entirety. As a non-limiting example, the polycation may include a cationic peptide or a polypeptide such as, but not limited to, polylysine, polyornithine and/or polyarginine and the cationic peptides described in International Pub. No. WO2012013326 or US Patent Pub. No. US20130142818; each of which is herein incorporated by reference in its entirety. In another embodiment, the RNA vaccines may be formulated in a lipid-polycation complex which may further include a non-cationic lipid such as, but not limited to, cholesterol or dioleoyl phosphatidylethanolamine (DOPE).

The RNA vaccines may be formulated in an aminoalcohol lipidoid. Aminoalcohol lipidoids which may be used in the present invention may be prepared by the methods described in U.S. Pat. No. 8,450,298, herein incorporated by reference in its entirety. The liposome formulation may be influenced by, but not limited to, the selection of the cationic lipid component, the degree of cationic lipid saturation, the nature of the PEGylation, ratio of all components and biophysical parameters such as size. In one example by Semple et al. (Semple et al. Nature Biotech. 2010 28:172-176; herein incorporated by reference in its entirety), the liposome formulation was composed of 57.1% cationic lipid, 7.1% dipalmitoylphosphatidylcholine, 34.3% cholesterol, and 1.4% PEG-c-DMA. As another example, changing the composition of the cationic lipid could more effectively deliver siRNA to various antigen presenting cells (Basha et al. Mol Ther. 2011 19:2186-2200; herein incorporated by reference in its entirety). In some embodiments, liposome formulations may comprise from about 35 to about 45% cationic lipid, from about 40% to about 50% cationic lipid, from about 50% to about 60% cationic lipid and/or from about 55% to about 65% cationic lipid. In some embodiments, the ratio of lipid to mRNA in liposomes may be from about about 5:1 to about 20:1, from about 10:1 to about 25:1, from about 15:1 to about 30:1 and/or at least 30:1. In some embodiments, the ratio of PEG in the lipid nanoparticle (LNP) formulations may be increased or decreased and/or the carbon chain length of the PEG lipid may be modified from C 14 to C18 to alter the pharmacokinetics and/or biodistribution of the LNP formulations. As a non-limiting example, LNP formulations may contain from about 0.5% to about 3.0%, from about 1.0% to about 3.5%, from about 1.5% to about 4.0%, from about 2.0% to about 4.5%, from about 2.5% to about 5.0% and/or from about 3.0% to about 6.0% of the lipid molar ratio of PEG-c-DOMG (R-3-[(co-methoxy-poly(ethyleneglycol) 2000) carbamoyl)]-1,2-dimyristyloxypropyl-3-amine) (also referred to herein as PEG-DOMG) as compared to the cationic lipid, DSPC and cholesterol. In another embodiment the PEG-c-DOMG may be replaced with a PEG lipid such as, but not limited to, PEG-DSG(1,2-Distearoyl-sn-glycerol, methoxypolyethylene glycol), PEG-DMG(1,2-Dimyristoyl-sn-glycerol) and/or PEG-DPG(1,2-Dipalmitoyl-sn-glycerol, methoxypolyethylene glycol). The cationic lipid may be selected from any lipid known in the art such as, but not limited to, DLin-MC3-DMA, D Lin-DMA, C 12-200 and DLin-KC2-DMA.

The RNA vaccines may be formulated in a lipid nanoparticle such as those described in International Publication No. WO2012170930, the contents of which is herein incorporated by reference in its entirety.

The RNA vaccine formulation comprising the polynucleotide may be a nanoparticle which may comprise at least one lipid. The lipid may be selected from, but is not limited to, DLin-DMA, DLin-K-DMA, 98N12-5, C 12-200, DLin-MC3-DMA, DLin-KC2-DMA, DODMA, PLGA, PEG, PEG-DMG, PEGylated lipids and amino alcohol lipids. In another aspect, the lipid may be a cationic lipid such as, but not limited to, DLin-DMA, DLin-D-DMA, DLin-MC3-DMA, DLin-KC2-DMA, DODMA and amino alcohol lipids. The amino alcohol cationic lipid may be the lipids described in and/or made by the methods described in US Patent Publication No. US20130150625, herein incorporated by reference in its entirety. As a non-limiting example, the cationic lipid may be 2-amino-3-[(9Z,12Z)-octadeca-9, 12-dien-1-yloxy]-2-{[(9Z,2Z)-octadeca-9, 12-dien-1-yloxy]methyl jpropan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in US20130150625); or any pharmaceutically acceptable salt or stereoisomer thereof.

Lipid nanoparticle formulations typically comprise a lipid, in particular, an ionizable cationic lipid, for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), and further comprise a neutral lipid, a sterol and a molecule capable of reducing particle aggregation, for example a PEG or PEG-modified lipid.

The lipid nanoparticle formulation may consist essentially of (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319); (ii) a neutral lipid selected from DSPC, DPPC, POPC, DOPE and SM; (iii) a sterol, e.g., cholesterol; and (iv) a PEG-lipid, e.g., PEG-DMG or PEG-cDMA, in a molar ratio of about 20-60% cationic lipid:5-25% neutral lipid:25-55% sterol; 0.5-15% PEG-lipid. In one embodiment, the formulation includes from about 25% to about 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., from about 35 to about 65%, from about 45 to about 65%, about 60%, about 57.5%, about 50% or about 40% on a molar basis.

In one embodiment, the formulation includes from about 0.5% to about 15% on a molar basis of the neutral lipid e.g., from about 3 to about 12%, from about 5 to about 10% or about 15%, about 10%, or about 7.5% on a molar basis. Exemplary neutral lipids include, but are not limited to, DSPC, POPC, DPPC, DOPE and SM. In one embodiment, the formulation includes from about 5% to about 50% on a molar basis of the sterol (e.g., about 15 to about 45%, about 20 to about 40%, about 40%, about 38.5%, about 35%, or about 31% on a molar basis. An exemplary sterol is cholesterol. In one embodiment, the formulation includes from about 0.5% to about 20% on a molar basis of the PEG or PEG-modified lipid (e.g., about 0.5 to about 10%, about 0.5 to about 5%, about 1.5%, about 0.5%, about 1.5%, about 3.5%, or about 5% on a molar basis. In one embodiment, the PEG or PEG modified lipid comprises a PEG molecule of an average molecular weight of 2,000 Da. In other embodiments, the PEG or PEG modified lipid comprises a PEG molecule of an average molecular weight of less than 2,000, for example around 1,500 Da, around 1,000 Da, or around 500 Da. Exemplary PEG-modified lipids include, but are not limited to, PEG-distearoyl glycerol (PEG-DMG) (also referred herein as PEG-C14 or C14-PEG), PEG-CDMA (further discussed in Reyes et al. J. Controlled Release, 107, 276-287 (2005) the contents of which are herein incorporated by reference in its entirety)

In one embodiment, the formulations include 25-75% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 0.5-15% of the neutral lipid, 5-50% of the sterol, and 0.5-20% of the PEG or PEG-modified lipid on a molar basis. In one embodiment, the formulations include 35-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 3-12% of the neutral lipid, 15-45% of the sterol, and 0.5-10% of the PEG or PEG-modified lipid on a molar basis.

In one embodiment, the formulations include 45-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 5-10% of the neutral lipid, 25-40% of the sterol, and 0.5-10% of the PEG or PEG-modified lipid on a molar basis.

In one embodiment, the formulations include about 60% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 7.5% of the neutral lipid, about 31% of the sterol, and about 1.5% of the PEG or PEG-modified lipid on a molar basis.

In one embodiment, the formulations include about 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 10% of the neutral lipid, about 38.5% of the sterol, and about 1.5% of the PEG or PEG-modified lipid on a molar basis.

In one embodiment, the formulations include about 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 10% of the neutral lipid, about 35% of the sterol, about 4.5% or about 5% of the PEG or PEG-modified lipid, and about 0.5% of the targeting lipid on a molar basis.

In one embodiment, the formulations of include about 40% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 15% of the neutral lipid, about 40% of the sterol, and about 5% of the PEG or PEG-modified lipid on a molar basis.

In one embodiment, the formulations include about 57.2% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 7.1% of the neutral lipid, about 34.3% of the sterol, and about 1.4% of the PEG or PEG-modified lipid on a molar basis.

In one embodiment, the formulations include about 57.5% of a cationic lipid selected from the PEG lipid is PEG-CDMA (PEG-CDMA is further discussed in Reyes et al. (J. Controlled Release, 107, 276-287 (2005), the contents of which are herein incorporated by reference in its entirety), about 7.5% of the neutral lipid, about 31.5% of the sterol, and about 3.5% of the PEG or PEG-modified lipid on a molar basis. In preferred embodiments, lipid nanoparticle formulation consists essentially of a lipid mixture in molar ratios of about 20-70% cationic lipid:5-45% neutral lipid:20-55% cholesterol: 0.5-15% PEG-modified lipid; more preferably in a molar ratio of about 20-60% cationic lipid:5-25% neutral lipid:25-55% cholesterol: 0.5-15% PEG-modified lipid. In particular embodiments, the molar lipid ratio is approximately 50/10/38.5/1.5 (mol % cationic lipid/neutral lipid, e.g., DSPC/Chol/PEG-modified lipid, e.g., PEG-DMG, PEG-DSG or PEG-DPG), 57.2/7.1134.3/1.4 (mol % cationic lipid/neutral lipid, e.g., DPPC/Chol/PEG-modified lipid, e.g., PEG-cDMA), 40/15/40/5 (mol % cationic lipid/neutral lipid, e.g., DSPC/Chol/PEG-modified lipid, e.g., PEG-DMG), 50/10/35/4.5/0.5 (mol % cationic lipid/neutral lipid, e.g., DSPC/Chol/PEG-modified lipid, e.g., PEG-DSG), 50/10/35/5 (cationic lipid/neutral lipid, e.g., DSPC/Chol/PEG-modified lipid, e.g., PEG-DMG), 40/10/40/10 (mol % cationic lipid/neutral lipid, e.g., DSPC/Chol/PEG-modified lipid, e.g., PEG-DMG or PEG-cDMA), 35/15/40/10 (mol % cationic lipid/neutral lipid, e.g., DSPC/Chol/PEG-modified lipid, e.g., PEG-DMG or PEG-cDMA) or 52/13/30/5 (mol % cationic lipid/neutral lipid, e.g., DSPC/Chol/PEG-modified lipid, e.g., PEG-DMG or PEG-cDMA).

Exemplary lipid nanoparticle compositions and methods of making same are described, for example, in Semple et al. (2010) Nat. Biotechnol. 28:172-176; Jayarama et al. (2012), Angew. Chem. Int. Ed., 51:8529-8533; and Maier et al. (2013) Molecular Therapy 21, 1570-1578 (the contents of each of which are incorporated herein by reference in their entirety).

In one embodiment, the lipid nanoparticle formulations described herein may comprise a cationic lipid, a PEG lipid and a structural lipid and optionally comprise a non-cationic lipid. As a non-limiting example, the lipid nanoparticle may comprise about 40-60% of cationic lipid, about 5-15% of a non-cationic lipid, about 1-2% of a PEG lipid and about 30-50% of a structural lipid. As another non-limiting example, the lipid nanoparticle may comprise about 50% cationic lipid, about 10% non-cationic lipid, about 1.5% PEG lipid and about 38.5% structural lipid. As yet another non-limiting example, the lipid nanoparticle may comprise about 55% cationic lipid, about 10% non-cationic lipid, about 2.5% PEG lipid and about 32.5% structural lipid. In one embodiment, the cationic lipid may be any cationic lipid described herein such as, but not limited to, DLin-KC2-DMA, DLin-MC3-DMA and L319.

In one embodiment, the lipid nanoparticle formulations described herein may be 4 component lipid nanoparticles. The lipid nanoparticle may comprise a cationic lipid, a non-cationic lipid, a PEG lipid and a structural lipid. As a non-limiting example, the lipid nanoparticle may comprise about 40-60% of cationic lipid, about 5-15% of a non-cationic lipid, about 1-2% of a PEG lipid and about 30-50% of a structural lipid. As another non-limiting example, the lipid nanoparticle may comprise about 50% cationic lipid, about 10% non-cationic lipid, about 1.5% PEG lipid and about 38.5% structural lipid. As yet another non-limiting example, the lipid nanoparticle may comprise about 55% cationic lipid, about 10% non-cationic lipid, about 2.5% PEG lipid and about 32.5% structural lipid. In one embodiment, the cationic lipid may be any cationic lipid described herein such as, but not limited to, DLin-KC2-DMA, DLin-MC3-DMA and L319.

In one embodiment, the lipid nanoparticle formulations described herein may comprise a cationic lipid, a non-cationic lipid, a PEG lipid and a structural lipid. As a non-limiting example, the lipid nanoparticle comprise about 50% of the cationic lipid DLin-KC2-DMA, about 10% of the non-cationic lipid DSPC, about 1.5% of the PEG lipid PEG-DOMG and about 38.5% of the structural lipid cholesterol. As a non-limiting example, the lipid nanoparticle comprise about 50% of the cationic lipid DLin-MC3-DMA, about 10% of the non-cationic lipid DSPC, about 1.5% of the PEG lipid PEG-DOMG and about 38.5% of the structural lipid cholesterol. As a non-limiting example, the lipid nanoparticle comprise about 50% of the cationic lipid DLin-MC3-DM A, about 10% of the non-cationic lipid DSPC, about 1.5% of the PEG lipid PEG-DMG and about 38.5% of the structural lipid cholesterol. As yet another non-limiting example, the lipid nanoparticle comprise about 55% of the cationic lipid L319, about 10% of the non-cationic lipid DSPC, about 2.5% of the PEG lipid PEG-DMG and about 32.5% of the structural lipid cholesterol.

In one embodiment, the cationic lipid may be selected from, but not limited to, a cationic lipid described in International Publication Nos. WO2012040184, WO2011153120, WO2011149733, WO2011090965, WO2011043913, WO2011022460, WO2012061259, WO2012054365, WO2012044638, WO2010080724, WO201021865, WO2008103276, WO2013086373 and WO2013086354, U.S. Pat. Nos. 7,893,302, 7,404,969, 8,283,333, and 8,466,122 and US Patent Publication No. US20100036115, US20120202871, US20130064894, US20130129785, US20130150625, US20130178541 and US20130225836; the contents of each of which are herein incorporated by reference in their entirety. In another embodiment, the cationic lipid may be selected from, but not limited to, formula A described in International Publication Nos. WO2012040184, WO2011153120, WO2011149733, WO2011090965, WO2011043913, WO2011022460, WO2012061259, WO2012054365, WO2012044638 and WO2013116126 or US Patent Publication No. US20130178541 and US20130225836; the contents of each of which is herein incorporated by reference in their entirety. In yet another embodiment, the cationic lipid may be selected from, but not limited to, formula CLI-CLXXIX of International Publication No. WO2008103276, formula CLI-CLXXIX of U.S. Pat. No. 7,893,302, formula CLI-CLXXXXII of U.S. Pat. No. 7,404,969 and formula I-VI of US Patent Publication No. US20100036115, formula I of US Patent Publication No US20130123338; each of which is herein incorporated by reference in their entirety. As a non-limiting example, the cationic lipid may be selected from (20Z,23Z)—N,N-dimethylnonacosa-20,23-dien-10-amine, (17Z,20Z)—N,N-dimemylhexacosa-17,20-dien-9-amine, (1Z,19Z)—N5N-dimethylpentacosa-16,19-dien-8-amine, (13Z,16Z)—N,N-dimethyldocosa-13,16-dien-5-amine, (12Z,15Z)—N,N-dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)—N,N-dimethylheptacosa-18,21-dien-10-amine, (15Z,18Z)—N,N-dimethyltetracosa-15,18-dien-5-amine, (14Z,17Z)—N,N-dimethyltricosa-14,17-dien-4-amine, (19Z,22Z)—N,N-dimeihyloctacosa-19,22-dien-9-amine, (18Z,21 Z)—N,N-dimethylheptacosa-18,21-dien-8-amine, (17Z,20Z)—N,N-dimethylhexacosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21 Z,24Z)—N,N-dimethyltriaconta-21,24-dien-9-amine, (18Z)—N,N-dimetylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methylnonacosa-20,23-dien-10-amine, 1-[(HZ,14Z)-1-nonylicosa-11,14-dien-1-yl]pyrrolidine, (20Z)—N,N-dimethylheptacos-20-en-10-amine, (15Z)—N,N-dimethyl eptacos-15-en-10-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-10-amine, (22Z)—N,N-dimethylhentriacont-22-en-10-amine, (16Z)—N,N-dimethylpentacos-16-en-8-amine, (12Z, 15Z)—N,N-dimethyl-2-nonylhenicosa-12, 15-dien-1-amine, (13Z, 16Z)—N,N-dimethyl-3-nonyldocosa-13,16-dien-1-amine, N,N-dimethyl-1-[(1S,2R)-2-octylcyclopropyl] eptadecan-8-amine, 1-[(1 S,2R)-2-hexylcyclopropyl]-N,N-dimethylnonadecan-10-amine, N,N-dimethyl-1-[(IS,2R)-2-octylcyclopropyl] nonadecan-10-amine, N,N-dimethyl-21-[(IS,2R)-2-octylcyclopropyl] henicosan-10-amine,N,N-dimethyl-1-[(1 S,2S)-2-{[(1R,2R)-2-pentylcyclopropyl]methyl} cyclopropyl] nonadecan-10-amine,N,N-dimethyl-1-[(1 S,2R)-2-octylcyclopropyl] hexadecan-8-amine, N,N-dimethyl-[(1R,2S)-2-undecyIcyclopropyl] tetradecan-5-amine, N,N-dimethyl-3-{7-[(IS,2R)-2-octylcyclopropyl] heptyl} dodecan-1-amine, 1-[(1R,2S)-2-hepty lcyclopropyl]-N,N-dimethyloctadecan-9-amine, 1-[(1 S,2R)-2-decylcyclopropyl]-N,N-dimethylpentadecan-6-amine, N,N-dimethyl-1-[(IS,2R)-2-octylcyclopropyl] pentadecan-8-amine, R—N,N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z, 12Z)-octadeca-9, 12-dien-1-yloxy]-1-[(octyloxy)methyl]ethyl} azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2S)-1-[(1 1Z,14Z)-icosa-11,14-dien-1-yloxy]-N,N-dimethyl-3-(pentyloxy)propan-2-amine, (2S)-1-(hexyloxy)-3-[(1 1Z,14Z)-icosa-11,14-dien-1-yloxy]-N,N-dimethylpropan-2-amine, 1-[(1 1Z,14Z)-icosa-11,14-dien-1-yloxy]-N,N-dimethyl-3-(octyloxy)propan-2-amine, 1-[(13Z, 16Z)-docosa-13, 16-dien-1-yloxy]-N,N-dimethyl-3-(octyloxy)propan-2-amine, (2S)-1-[(13Z, 16Z)-docosa-13, 16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H (1-metoylo ctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl} cyclopropyl] octyl} oxy)propan-2-amine, N,N-dimethyl-1-{[8-(2-oc 1 ylcyclopropyl)octyl]oxy}-3-(octyloxy)propan-2-amine and (11E,20Z,23Z)—N,N-dimethylnonacosa-11,20,2-trien-10-amine or a pharmaceutically acceptable salt or stereoisomer thereof.

In one embodiment, the lipid may be a cleavable lipid such as those described in International Publication No. WO2012170889, herein incorporated by reference in its entirety. In another embodiment, the lipid may be a cationic lipid such as, but not limited to, Formula (I) of U.S. Patent Application No. US20130064894, the contents of which are herein incorporated by reference in its entirety.

The cationic lipid may be synthesized by methods known in the art and/or as described in International Publication Nos. WO2012040184, WO2011153120, WO2011149733, WO2011090965, WO2011043913, WO2011022460, WO2012061259, WO2012054365, WO2012044638, WO2010080724, WO201021865, WO2013086373 and WO2013086354; the contents of each of which are herein incorporated by reference in their entirety.

The cationic lipid may be a trialkyl cationic lipid. Non-limiting examples of trialkyl cationic lipids and methods of making and using the trialkyl cationic lipids are described in International Patent Publication No. WO2013126803, the contents of which are herein incorporated by reference in its entirety.

LNP formulations of the RNA vaccines may contain PEG-c-DOMG at 3% lipid molar ratio. In another embodiment, the LNP formulations of the RNA vaccines may contain PEG-c-DOMG at 1.5% lipid molar ratio. Pharmaceutical compositions of the RNA vaccines may include at least one of the PEGylated lipids described in International Publication No. WO2012099755, the contents of which is herein incorporated by reference in its entirety.

The LNP formulation may contain PEG-DMG 2000 (1,2-dimyristoyl-sn-glycero-3-phophoethanolamine-N-[methoxy (polyethylene glycol)-2000). In one embodiment, the LNP formulation may contain PEG-DMG 2000, a cationic lipid known in the art and at least one other component. In another embodiment, the LNP formulation may contain PEG-DMG 2000, a cationic lipid known in the art, DSPC and cholesterol. As a non-limiting example, the LNP formulation may contain PEG-DMG 2000, DLin-DMA, DSPC and cholesterol. As another non-limiting example the LNP formulation may contain PEG-DMG 2000, DLin-DMA, DSPC and cholesterol in a molar ratio of 2:40:10:48 (see e.g., Geall et al., Nonviral delivery of self-amplifying RNA vaccines, PNAS 2012; PMID: 22908294; herein incorporated by reference in its entirety).

An LNP formulation may be formulated by the methods described in International Publication Nos. WO2011127255 or WO2008103276, the contents of each of which is herein incorporated by reference in their entirety. As a non-limiting example, the RNA vaccines described herein may be encapsulated in LNP formulations as described in WO2011127255 and/or WO2008103276; each of which is herein incorporated by reference in their entirety.

The RNA vaccines described herein may be formulated in a nanoparticle to be delivered by a parenteral route as described in U.S. Pub. No. US20120207845; the contents of which are herein incorporated by reference in its entirety. In one embodiment, the RNA vaccines may be formulated in a lipid nanoparticle made by the methods described in US Patent Publication No US20130156845 or International Publication No WO2013093648 or WO2012024526, each of which is herein incorporated by reference in its entirety. The lipid nanoparticles described herein may be made in a sterile environment by the system and/or methods described in US Patent Publication No. US20130164400, herein incorporated by reference in its entirety.

An LNP formulation may be formulated in a nanoparticle such as a nucleic acid-lipid particle described in U.S. Pat. No. 8,492,359, the contents of which are herein incorporated by reference in its entirety. As a non-limiting example, the lipid particle may comprise one or more active agents or therapeutic agents; one or more cationic lipids comprising from about 50 mol % to about 85 mol % of the total lipid present in the particle; one or more non-cationic lipids comprising from about 13 mol % to about 49.5 mol % of the total lipid present in the particle; and one or more conjugated lipids that inhibit aggregation of particles comprising from about 0.5 mol % to about 2 mol % of the total lipid present in the particle. The nucleic acid in the nanoparticle may be the polynucleotides described herein and/or are known in the art.

An LNP formulation may be formulated by the methods described in International Publication Nos. WO2011127255 or WO2008103276, the contents of each of which are herein incorporated by reference in their entirety. As a non-limiting example, modified RNA described herein may be encapsulated in LNP formulations as described in WO2011127255 and/or WO2008103276; the contents of each of which are herein incorporated by reference in their entirety. LNP formulations described herein may comprise a polycationic composition. As a non-limiting example, the polycationic composition may be selected from formula 1-60 of US Patent Publication No. US20050222064; the content of which is herein incorporated by reference in its entirety.

LNP formulations comprising a polycationic composition may be used for the delivery of the modified RNA described herein in vivo and/or in vitro. In one embodiment, the LNP formulations described herein may additionally comprise a permeability enhancer molecule. Non-limiting permeability enhancer molecules are described in US Patent Publication No. US20050222064; the content of which is herein incorporated by reference in its entirety.

The RNA vaccine pharmaceutical compositions may be formulated in liposomes such as, but not limited to, DiLa2 liposomes (Marina Biotech, Bothell, WA), SMARTICLES® (Marina Biotech, Bothell, WA), neutral DOPC (1,2-dioleoyl-sn-glycero-3-phosphocholine) based liposomes (e.g., siRNA delivery for ovarian cancer (Landen et al. Cancer Biology & Therapy 2006 5(12) 1708-1713); herein incorporated by reference in its entirety) and hyaluronan-coated liposomes (Quiet Therapeutics, Israel). In one embodiment, the RNA vaccines may be formulated in a lyophilized gel-phase liposomal composition as described in US Publication No. US2012060293, herein incorporated by reference in its entirety.

The RNA vaccine may be associated with a cationic or polycationic compounds, including protamine, nucleoline, spermine or spermidine, or other cationic peptides or proteins, such as poly-L-lysine (PLL), polyarginine, basic polypeptides, cell penetrating peptides (CPPs), including HIV-binding peptides, HIV-1 Tat (HIV), Tat-derived peptides, Penetratin, VP22 derived or analog peptides, Pestivirus Erns, HSV, VP22 (Herpes simplex), MAP, KALA or protein transduction domains (PTDs), PpT620, prolin-rich peptides, arginine-rich peptides, lysine-rich peptides, MPG-peptide(s), Pep-1, L-oligomers, Calcitonin peptide(s), Antennapedia-derived peptides (particularly from Drosophila antennapedia), pAntp, plsl, FGF, Lactoferrin, Transportan, Buforin-2, Bac715-24, SynB, SynB (1), pVEC, hCT-derived peptides, SAP, histones, cationic polysaccharides, for example chitosan, polybrene, cationic polymers, e.g. polyethyleneimine (PEI), cationic lipids, e.g. DOTMA: [1-(2,3-sioleyloxy)propyl)]-N,N,N-trimethylammonium chloride, DMRIE, di-C14-amidine, DOTIM, SAINT, DC-Choi, BGTC, CTAP, DOPC, DODAP, DOPE: Dioleylphosphatidylethanol-amine, DOSPA, DODAB, DOIC, DMEPC, DOGS: Dioctadecylamidoglicylspermin, DIMRI: Dimyristooxypropyl dimethyl hydroxyethyl ammonium bromide, DOTAP: dioleoyloxy-3-(trimethylammonio)propane, DC-6-14:0,0-ditetradecanoyl-N-.alpha.-trimethylammonioacetyl)diethanolamine chloride, CLIP 1: rac-[(2,3-dioctadecyloxypropyl)(2-hydroxyethyl)]-dimethylammonium chloride, CLIP6: rac-[2(2,3-dihexadecyloxypropyloxymethyloxy)ethyl]-trimethylammonium, CLIP9: rac-[2(2,3-dihexadecyloxypropyloxysuccinyloxy)ethyl]-trimethylammo-nium, oligofectamine, or cationic or polycationic polymers, e.g. modified polyaminoacids, such as beta-aminoacid-polymers or reversed polyamides, etc., modified polyethylenes, such as PVP (poly(N-ethyl-4-vinylpyridinium bromide)), etc., modified acrylates, such as pDMAEMA (poly(dimethylaminoethyl methylacrylate)), etc., modified amidoamines such as pAMAM (poly(amidoamine)), etc., modified polybetaminoester (PBAE), such as diamine end modified 1,4 butanediol diacrylate-co-5-amino-1-pentanol polymers, etc., dendrimers, such as polypropylamine dendrimers or pAMAM based dendrimers, etc., polyimine(s), such as PEL poly(ethyleneimine), poly(propyleneimine), etc., polyallylamine, sugar backbone based polymers, such as cyclodextrin based polymers, dextran based polymers, chitosan, etc., silan backbone based polymers, such as PMOXA-PDMS copolymers, etc., blockpolymers consisting of a combination of one or more cationic blocks (e.g. selected from a cationic polymer as mentioned above) and of one or more hydrophilic or hydrophobic blocks (e.g. polyethyleneglycole); etc.

Alternatively the RNA vaccine is not associated with a cationic or polycationic compounds.

The nanoparticle formulations may comprise a phosphate conjugate. The phosphate conjugate may increase in vivo circulation times and/or increase the targeted delivery of the nanoparticle. Phosphate conjugates for use with the present invention may be made by the methods described in International Application No. WO2013033438 or US Patent Publication No. US20130196948, the contents of each of which are herein incorporated by reference in its entirety. As a non-limiting example, the phosphate conjugates may include a compound of any one of the formulas described in International Application No. WO2013033438, herein incorporated by reference in its entirety.

The nanoparticle formulation may comprise a polymer conjugate. The polymer conjugate may be a water soluble conjugate. The polymer conjugate may have a structure as described in U.S. patent application No. 20130059360, the contents of which are herein incorporated by reference in its entirety. In one aspect, polymer conjugates with the polynucleotides of the present invention may be made using the methods and/or segmented polymeric reagents described in U.S. patent application No. 20130072709, herein incorporated by reference in its entirety. In another aspect, the polymer conjugate may have pendant side groups comprising ring moieties such as, but not limited to, the polymer conjugates described in US Patent Publication No. US20130196948, the contents of which is herein incorporated by reference in its entirety.

The nanoparticle formulations may comprise a conjugate to enhance the delivery of nanoparticles of the present invention in a subject. Further, the conjugate may inhibit phagocytic clearance of the nanoparticles in a subject. In one aspect, the conjugate may be a “self peptide designed from the human membrane protein CD47 (e.g., the “self particles described by Rodriguez et al (Science 2013 339, 971-975), herein incorporated by reference in its entirety). As shown by Rodriguez et al. the self peptides delayed macrophage-mediated clearance of nanoparticles which enhanced delivery of the nanoparticles. In another aspect, the conjugate may be the membrane protein CD47 (e.g., see Rodriguez et al. Science 2013 339, 971-975, herein incorporated by reference in its entirety). Rodriguez et al. showed that, similarly to “self peptides, CD47 can increase the circulating particle ratio in a subject as compared to scrambled peptides and PEG coated nanoparticles.

RNA vaccines may be formulated in nanoparticles which comprise a conjugate to enhance the delivery of the nanoparticles of the present invention in a subject. The conjugate may be the CD47 membrane or the conjugate may be derived from the CD47 membrane protein, such as the “self peptide described previously. In another aspect the nanoparticle may comprise PEG and a conjugate of CD47 or a derivative thereof. In yet another aspect, the nanoparticle may comprise both the “self peptide described above and the membrane protein CD47. A “self peptide and/or CD47 protein may be conjugated to a viruslike particle or pseudovirion, as described herein for delivery of the RNA vaccines.

The formulations herein may include RNA vaccine pharmaceutical compositions comprising the polynucleotides of the present invention and a conjugate which may have a degradable linkage. Non-limiting examples of conjugates include an aromatic moiety comprising an ionizable hydrogen atom, a spacer moiety, and a water-soluble polymer. As a non-limiting example, pharmaceutical compositions comprising a conjugate with a degradable linkage and methods for delivering such pharmaceutical compositions are described in US Patent Publication No. US20130184443, the contents of which are herein incorporated by reference in its entirety.

The nanoparticle formulations may be a carbohydrate nanoparticle comprising a carbohydrate carrier and a RNA vaccine. As a non-limiting example, the carbohydrate carrier may include, but is not limited to, an anhydride-modified phytoglycogen or glycogen-type material, phtoglycogen octenyl succinate, phytoglycogen beta-dextrin, anhydride-modified phytoglycogen beta-dextrin. (See e.g., International Publication No. WO2012109121; the contents of which are herein incorporated by reference in its entirety).

Nanoparticle formulations may be coated with a surfactant or polymer in order to improve the delivery of the particle. In one embodiment, the nanoparticle may be coated with a hydrophilic coating such as, but not limited to, PEG coatings and/or coatings that have a neutral surface charge. The hydrophilic coatings may help to deliver nanoparticles with larger payloads such as, but not limited to, RNA vaccines within the central nervous system. As a non-limiting example nanoparticles comprising a hydrophilic coating and methods of making such nanoparticles are described in US Patent Publication No. US20130183244, the contents of which are herein incorporated by reference in its entirety. In one embodiment, the lipid nanoparticles of the present invention may be hydrophilic polymer particles. Non-limiting examples of hydrophilic polymer particles and methods of making hydrophilic polymer particles are described in US Patent Publication No. US20130210991, the contents of which are herein incorporated by reference in its entirety. In another embodiment, the lipid nanoparticles of the present invention may be hydrophobic polymer particles.

Lipid nanoparticle formulations may be improved by replacing the cationic lipid with a biodegradable cationic lipid which is known as a rapidly eliminated lipid nanoparticle (reLNP). Ionizable cationic lipids, such as, but not limited to, DLinDMA, DLin-KC2-DMA, and DLin-MC3-DMA, have been shown to accumulate in plasma and tissues over time and may be a potential source of toxicity. The rapid metabolism of the rapidly eliminated lipids can improve the tolerability and therapeutic index of the lipid nanoparticles by an order of magnitude from a 1 mg/kg dose to a 10 mg/kg dose in rat. Inclusion of an enzymatically degraded ester linkage can improve the degradation and metabolism profile of the cationic component, while still maintaining the activity of the reLNP formulation. The ester linkage can be internally located within the lipid chain or it may be terminally located at the terminal end of the lipid chain. The internal ester linkage may replace any carbon in the lipid chain. In one embodiment, the internal ester linkage may be located on either side of the saturated carbon.

In one embodiment, an immune response may be elicited by delivering a lipid nanoparticle which may include a nanospecies, a polymer and an immunogen. (U.S. Publication No. 20120189700 and International Publication No. WO2012099805; each of which is herein incorporated by reference in their entirety). The polymer may encapsulate the nanospecies or partially encapsulate the nanospecies. The immunogen may be a recombinant protein, a modified RNA and/or a polynucleotide described herein. In one embodiment, the lipid nanoparticle may be formulated for use in a vaccine such as, but not limited to, against a pathogen. Lipid nanoparticles may be engineered to alter the surface properties of particles so the lipid nanoparticles may penetrate the mucosal barrier. Mucus is located on mucosal tissue such as, but not limited to, oral (e.g., the buccal and esophageal membranes and tonsil tissue), ophthalmic, gastrointestinal (e.g., stomach, small intestine, large intestine, colon, rectum), nasal, respiratory (e.g., nasal, pharyngeal, tracheal and bronchial membranes), genital (e.g., vaginal, cervical and urethral membranes). Nanoparticles larger than 10-200 nm which are preferred for higher drug encapsulation efficiency and the ability to provide the sustained delivery of a wide array of drugs have been thought to be too large to rapidly diffuse through mucosal barriers. Mucus is continuously secreted, shed, discarded or digested and recycled so most of the trapped particles may be removed from the mucosal tissue within seconds or within a few hours. Large polymeric nanoparticles (200 nm-500 nm in diameter) which have been coated densely with a low molecular weight polyethylene glycol (PEG) diffused through mucus only 4 to 6-fold lower than the same particles diffusing in water (Lai et al. PNAS 2007 104(5): 1482-487; Lai et al. Adv Drug Deliv Rev. 2009 61(2): 158-171; each of which is herein incorporated by reference in their entirety). The transport of nanoparticles may be determined using rates of permeation and/or fluorescent microscopy techniques including, but not limited to, fluorescence recovery after photobleaching (FRAP) and high resolution multiple particle tracking (MPT). As a non-limiting example, compositions which can penetrate a mucosal barrier may be made as described in U.S. Pat. No. 8,241,670 or International Patent Publication No. WO2013110028, the contents of each of which are herein incorporated by reference in its entirety.

The lipid nanoparticle engineered to penetrate mucus may comprise a polymeric material (i.e. a polymeric core) and/or a polymer-vitamin conjugate and/or a tri-block copolymer. The polymeric material may include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates. The polymeric material may be biodegradable and/or biocompatible. Non-limiting examples of biocompatible polymers are described in International Patent Publication No. WO2013116804, the contents of which are herein incorporated by reference in its entirety. The polymeric material may additionally be irradiated. As a non-limiting example, the polymeric material may be gamma irradiated (See e.g., International App. No. WO201282165, herein incorporated by reference in its entirety). Non-limiting examples of specific polymers include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poly(ortho) esters, poly(butyric acid), poly(valeric acid), poly(lactide-co-caprolactone), PEG-PLGA-PEG and trimethylene carbonate, polyvinylpyrrolidone. The lipid nanoparticle may be coated or associated with a co-polymer such as, but not limited to, a block co-polymer (such as a branched polyether-polyamide block copolymer described in International Publication No. WO2013012476, herein incorporated by reference in its entirety), and (poly(ethylene glycol))-(poly(propylene oxide))-(poly(ethylene glycol)) triblock copolymer (see e.g., US Publication 20120121718 and US Publication 20100003337 and U.S. Pat. No. 8,263,665; each of which is herein incorporated by reference in their entirety). The co-polymer may be a polymer that is generally regarded as safe (GRAS) and the formation of the lipid nanoparticle may be in such a way that no new chemical entities are created. For example, the lipid nanoparticle may comprise poloxamers coating PLGA nanoparticles without forming new chemical entities which are still able to rapidly penetrate human mucus (Yang et al. Angew. Chem. Int. Ed. 2011 50:2597-2600; the contents of which are herein incorporated by reference in its entirety).

A non-limiting scalable method to produce nanoparticles which can penetrate human mucus is described by Xu et al. (See e.g., J Control Release 2013, 170(2): 279-86; the contents of which are herein incorporated by reference in its entirety). The vitamin of the polymer-vitamin conjugate may be vitamin E. The vitamin portion of the conjugate may be substituted with other suitable components such as, but not limited to, vitamin A, vitamin E, other vitamins, cholesterol, a hydrophobic moiety, or a hydrophobic component of other surfactants (e.g., sterol chains, fatty acids, hydrocarbon chains and alkylene oxide chains).

The lipid nanoparticle engineered to penetrate mucus may include surface altering agents such as, but not limited to, polynucleotides, anionic proteins (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin β4 dornase alfa, neltenexine, erdosteine) and various DNases including rhDNase. The surface altering agent may be embedded or enmeshed in the particle's surface or disposed (e.g., by coating, adsorption, covalent linkage, or other process) on the surface of the lipid nanoparticle. (see e.g., US Publication 20100215580 and US Publication 20080166414 and US20130164343; the contents of each of which is herein incorporated by reference in their entirety).

The mucus penetrating lipid nanoparticles may comprise at least one polynucleotide described herein. The polynucleotide may be encapsulated in the lipid nanoparticle and/or disposed on the surface of the particle. The polynucleotide may be covalently coupled to the lipid nanoparticle. Formulations of mucus penetrating lipid nanoparticles may comprise a plurality of nanoparticles. Further, the formulations may contain particles which may interact with the mucus and alter the structural and/or adhesive properties of the surrounding mucus to decrease mucoadhesion which may increase the delivery of the mucus penetrating lipid nanoparticles to the mucosal tissue.

The mucus penetrating lipid nanoparticles may be a hypotonic formulation comprising a mucosal penetration enhancing coating. The formulation may be hypotonic for the epithelium to which it is being delivered. Non-limiting examples of hypotonic formulations may be found in International Patent Publication No. WO2013110028, the contents of which are herein incorporated by reference in its entirety. In order to enhance the delivery through the mucosal barrier the RNA vaccine formulation may comprise or be a hypotonic solution. Hypotonic solutions were found to increase the rate at which mucoinert particles such as, but not limited to, mucus-penetrating particles, were able to reach the vaginal epithelial surface (See e.g., Ensign et al. Biomaterials 2013 34(28): 6922-9; the contents of which is herein incorporated by reference in its entirety). In one embodiment, the RNA vaccine is formulated as a lipoplex, such as, without limitation, the ATUPLEX™ system, the DACC system, the DBTC system and other siRNA-lipoplex technology from Silence Therapeutics (London, United Kingdom), STEMFECT™ from STEMGENT® (Cambridge, MA), and polyethylenimine (PEI) or protamine-based targeted and non-targeted delivery of nucleic acids acids (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344; Kaufmann et al. Microvasc Res 2010 80:286-293 Weide et al. J Immunother. 2009 32:498-507; Weide et al. J Immunother. 2008 31:180-188; Pascolo Expert Opin. Biol. Ther. 4:1285-1294; Fotin-Mleczek et al., 2011 J. Immunother. 34:1-15; Song et al., Nature Biotechnol. 2005, 23:709-717; Peer et al., Proc Natl Acad Sci USA. 2007 6; 104:4095-4100; deFougerolles Hum Gene Ther. 2008 19:125-132; all of which are incorporated herein by reference in its entirety).

Such formulations may also be constructed or compositions altered such that they passively or actively are directed to different cell types in vivo, including but not limited to hepatocytes, immune cells, tumor cells, endothelial cells, antigen presenting cells, and leukocytes (Akinc et al. Mol Ther. 2010 18:1357-1364; Song et al., Nat Biotechnol. 2005 23:709-717; Judge et al., J Clin Invest. 2009 119:661-673; Kaufmann et al., Microvasc Res 2010 80:286-293; Santel et al., Gene Ther 2006 13:1222-1234; Santel et al., Gene Ther 2006 13:1360-1370; Gutbier et al., Pulm Pharmacol. Ther. 2010 23:334-344; Basha et al., Mol. Ther. 2011 19:2186-2200; Fenske and Cullis, Expert Opin Drug Deliv. 2008 5:25-44; Peer et al., Science. 2008 319:627-630; Peer and Lieberman, Gene Ther. 2011 18:1127-1133; all of which are incorporated herein by reference in its entirety). One example of passive targeting of formulations to liver cells includes the DLin-DMA, DLin-KC2-DMA and DLin-MC3-DMA-based lipid nanoparticle formulations which have been shown to bind to apolipoprotein E and promote binding and uptake of these formulations into hepatocytes in vivo (Akinc et al. Mol Ther. 2010 18:1357-1364; herein incorporated by reference in its entirety). Formulations can also be selectively targeted through expression of different ligands on their surface as exemplified by, but not limited by, folate, transferrin, N-acetylgalactosamine (GalNAc), and antibody targeted approaches (Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; Peer and Lieberman, Gene Ther. 2011 18:1127-1133; all of which are incorporated herein by reference in its entirety).

The RNA vaccine may be formulated as a solid lipid nanoparticle. A solid lipid nanoparticle (SLN) may be spherical with an average diameter between 10 to 1000 nm. SLN possess a solid lipid core matrix that can solubilize lipophilic molecules and may be stabilized with surfactants and/or emulsifiers. In a further embodiment, the lipid nanoparticle may be a self-assembly lipid-polymer nanoparticle (see Zhang et al., ACS Nano, 2008, 2(8), pp 1696-1702; the contents of which are herein incorporated by reference in its entirety). As a non-limiting example, the SLN may be the SLN described in International Patent Publication No. WO2013105101, the contents of which are herein incorporated by reference in its entirety. As another non-limiting example, the SLN may be made by the methods or processes described in International Patent Publication No. WO2013105101, the contents of which are herein incorporated by reference in its entirety. I11

Liposomes, lipoplexes, or lipid nanoparticles may be used to improve the efficacy of polynucleotides directed protein production as these formulations may be able to increase cell transfection by the RNA vaccine; and/or increase the translation of encoded protein. One such example involves the use of lipid encapsulation to enable the effective systemic delivery of polyplex plasmid DNA (Heyes et al., Mol Ther. 2007 15:713-720; herein incorporated by reference in its entirety). The liposomes, lipoplexes, or lipid nanoparticles may also be used to increase the stability of the polynucleotide.

RNA vaccines of the present invention can also be formulated for controlled release and/or targeted delivery. As used herein, “controlled release” refers to a pharmaceutical composition or compound release profile that conforms to a particular pattern of release to effect a therapeutic outcome. In one embodiment, the RRNA vaccines may be encapsulated into a delivery agent described herein and/or known in the art for controlled release and/or targeted delivery. As used herein, the term “encapsulate” means to enclose, surround or encase. As it relates to the formulation of the compounds of the invention, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of the pharmaceutical composition or compound of the invention may be enclosed, surrounded or encased within the delivery agent. “Partially encapsulated” means that less than 10, 10, 20, 30, 40 50 or less of the pharmaceutical composition or compound of the invention may be enclosed, surrounded or encased within the delivery agent.

Advantageously, encapsulation may be determined by measuring the escape or the activity of the pharmaceutical composition or compound of the invention using fluorescence and/or electron micrograph. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the invention are encapsulated in the delivery agent. A controlled release formulation may include, but is not limited to, tri-block co-polymers. As a non-limiting example, the formulation may include two different types of tri-block co-polymers (International Pub. No. WO2012131104 and WO2012131106; the contents of each of which is herein incorporated by reference in its entirety).

RNA vaccines may be encapsulated into a lipid nanoparticle or a rapidly eliminated lipid nanoparticle and the lipid nanoparticles or a rapidly eliminated lipid nanoparticle may then be encapsulated into a polymer, hydrogel and/or surgical sealant described herein and/or known in the art. As a non-limiting example, the polymer, hydrogel or surgical sealant may be PLGA, ethylene vinyl acetate (EVAc), poloxamer, GELSITE® (Nanotherapeutics, Inc. Alachua, FL), HYLENEX® (Halozyme Therapeutics, San Diego CA), surgical sealants such as fibrinogen polymers (Ethicon Inc. Cornelia, GA), TISSELL® (Baxter International, Inc Deerfield, IL), PEG-based sealants, and COSEAL®(Baxter International, Inc Deerfield, IL).

The lipid nanoparticle may be encapsulated into any polymer known in the art which may form a gel when injected into a subject. As another non-limiting example, the lipid nanoparticle may be encapsulated into a polymer matrix which may be biodegradable.

The RNA vaccine formulation for controlled release and/or targeted delivery may also include at least one controlled release coating. Controlled release coatings include, but are not limited to, OPADRY®, polyvinylpyrrolidone/vinyl acetate copolymer, polyvinylpyrrolidone, hydroxypropyl methylcellulose, hydroxypropyl cellulose, hydroxyethyl cellulose, EUDRAGIT RL®, EUDRAGIT RS® and cellulose derivatives such as ethylcellulose aqueous dispersions (AQUACOAT® and SURELEASE®). In one embodiment, the RNA vaccine controlled release and/or targeted delivery formulation may comprise at least one degradable polyester which may contain polycationic side chains. Degradeable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer. The RNA vaccine controlled release and/or targeted delivery formulation comprising at least one polynucleotide may comprise at least one PEG and/or PEG related polymer derivatives as described in U.S. Pat. No. 8,404,222, herein incorporated by reference in its entirety.

The RNA vaccine controlled release delivery formulation comprising at least one polynucleotide may be the controlled release polymer system described in US20130130348, herein incorporated by reference in its entirety. RNA vaccines of the present invention may be encapsulated in a therapeutic nanoparticle, referred to herein as “therapeutic nanoparticle RRNA vaccines.” Therapeutic nanoparticles may be formulated by methods described herein and known in the art such as, but not limited to, International Pub Nos. WO2010005740, WO2010030763, WO2010005721, WO2010005723, WO2012054923, US Pub. Nos. US20110262491, US20100104645, US20100087337, US20100068285, US20110274759, US20100068286, US20120288541, US20130123351 and US20130230567 and U.S. Pat. Nos. 8,206,747, 8,293,276, 8,318,208 and 8,318,211; the contents of each of which are herein incorporated by reference in their entirety. In another embodiment, therapeutic polymer nanoparticles may be identified by the methods described in US Pub No. US20120140790, the contents of which is herein incorporated by reference in its entirety.

A therapeutic nanoparticle RNA vaccine may be formulated for sustained release. As used herein, “sustained release” refers to a pharmaceutical composition or compound that conforms to a release rate over a specific period of time. The period of time may include, but is not limited to, hours, days, weeks, months and years. As a non-limiting example, the sustained release nanoparticle may comprise a polymer and a therapeutic agent such as, but not limited to, the polynucleotides of the present invention (see International Pub No. 2010075072 and US Pub No. US20100216804, US20110217377 and US20120201859, each of which is herein incorporated by reference in their entirety). In another non-limiting example, the sustained release formulation may comprise agents which permit persistent bioavailability such as, but not limited to, crystals, macromolecular gels and/or particulate suspensions (see US Patent Publication No US20130150295, the contents of which is herein incorporated by reference in its entirety). In one embodiment, the therapeutic nanoparticle RNA vaccines may be formulated to be target specific. As a non-limiting example, the therapeutic nanoparticles may include a corticosteroid (see International Pub. No. WO2011084518; herein incorporated by reference in its entirety). As a non-limiting example, the therapeutic nanoparticles may be formulated in nanoparticles described in International Pub No. WO2008121949, WO2010005726, WO2010005725, WO2011084521 and US Pub No. US20100069426, US20120004293 and US20100104655, each of which is herein incorporated by reference in their entirety.

The nanoparticles of the present invention may comprise a polymeric matrix. As a non-limiting example, the nanoparticle may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof. In one embodiment, the therapeutic nanoparticle comprises a diblock copolymer. In one embodiment, the diblock copolymer may include PEG in combination with a polymer such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof. In another embodiment, the diblock copolymer may comprise the diblock copolymers described in European Patent Publication No. the contents of which are herein incorporated by reference in its entirety. In yet another embodiment, the diblock copolymer may be a high-X diblock copolymer such as those described in International Patent Publication No. WO2013120052, the contents of which are herein incorporated by reference in its entirety.

As a non-limiting example the therapeutic nanoparticle comprises a PLGA-PEG block copolymer (see US Pub. No. US20120004293 and U.S. Pat. No. 8,236,330, each of which is herein incorporated by reference in their entirety). In another non-limiting example, the therapeutic nanoparticle is a stealth nanoparticle comprising a diblock copolymer of PEG and PLA or PEG and PLGA (see U.S. Pat. No. 8,246,968 and International Publication No. WO2012166923, the contents of each of which are herein incorporated by reference in its entirety). In yet another non-limiting example, the therapeutic nanoparticle is a stealth nanoparticle or a target-specific stealth nanoparticle as described in US Patent Publication No. US20130172406, the contents of which are herein incorporated by reference in its entirety.

The therapeutic nanoparticle may comprise a multiblock copolymer (See e.g., U.S. Pat. Nos. 8,263,665 and 8,287,910 and US Patent Pub. No. US20130195987; the contents of each of which are herein incorporated by reference in its entirety). In yet another non-limiting example, the lipid nanoparticle comprises the block copolymer PEG-PLGA-PEG (see e.g., the thermosensitive hydrogel (PEG-PLGA-PEG) was used as a TGF-betal gene delivery vehicle in Lee et al. Thermosensitive Hydrogel as a Tgf-βî Gene Delivery Vehicle Enhances Diabetic Wound Healing. Pharmaceutical Research, 2003 20(12): 1995-2000; as a controlled gene delivery system in Li et al. Controlled Gene Delivery System Based on Thermosensitive Biodegradable Hydrogel. Pharmaceutical Research 2003 20(6): 884-888; and Chang et al., Non-ionic amphiphilic biodegradable PEG-PLGA-PEG copolymer enhances gene delivery efficiency in rat skeletal muscle. J Controlled Release. 2007 118:245-253; each of which is herein incorporated by reference in its entirety).

The RNA vaccines of the present invention may be formulated in lipid nanoparticles comprising the PEG-PLGA-PEG block copolymer. The therapeutic nanoparticle may comprise a multiblock copolymer (See e.g., U.S. Pat. Nos. 8,263,665 and 8,287,910 and US Patent Pub. No. US20130195987; the contents of each of which are herein incorporated by reference in its entirety). The block copolymers described herein may be included in a polyion complex comprising a non-polymeric micelle and the block copolymer. (See e.g., U.S. Pub. No. 20120076836; herein incorporated by reference in its entirety).

The therapeutic nanoparticle may comprise at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

The therapeutic nanoparticles may comprise at least one poly(vinyl ester) polymer. The poly(vinyl ester) polymer may be a copolymer such as a random copolymer. As a non-limiting example, the random copolymer may have a structure such as those described in International Application No. WO2013032829 or US Patent Publication No US20130121954, the contents of which are herein incorporated by reference in its entirety. In one aspect, the poly(vinyl ester) polymers may be conjugated to the polynucleotides described herein. In another aspect, the poly(vinyl ester) polymer which may be used in the present invention may be those described in, herein incorporated by reference in its entirety.

The therapeutic nanoparticle may comprise at least one diblock copolymer. The diblock copolymer may be, but it not limited to, a poly(lactic) acid-poly(ethylene)glycol copolymer (see e.g., International Patent Publication No. WO2013044219; herein incorporated by reference in its entirety). As a non-limiting example, the therapeutic nanoparticle may be used to treat cancer (see International publication No. WO2013044219; herein incorporated by reference in its entirety). In one embodiment, the therapeutic nanoparticles may comprise at least one cationic polymer described herein and/or known in the art.

The therapeutic nanoparticles may comprise at least one amine-containing polymer such as, but not limited to polylysine, polyethylene imine, poly(amidoamine) dendrimers, poly(beta-amino esters) (See e.g., U.S. Pat. No. 8,287,849; herein incorporated by reference in its entirety) and combinations thereof. In another embodiment, the nanoparticles described herein may comprise an amine cationic lipid such as those described in International Patent Application No. WO2013059496, the contents of which are herein incorporated by reference in its entirety. In one aspect the cationic lipids may have an amino-amine or an amino-amide moiety.

The therapeutic nanoparticles may comprise at least one degradable polyester which may contain polycationic side chains. Degradeable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

The therapeutic nanoparticle may include a conjugation of at least one targeting ligand. The targeting ligand may be any ligand known in the art such as, but not limited to, a monoclonal antibody. (Kirpotin et al, Cancer Res. 2006 66:6732-6740; herein incorporated by reference in its entirety).

The therapeutic nanoparticle may be formulated in an aqueous solution which may be used to target cancer (see International Pub No. WO2011084513 and US Pub No. US20110294717, each of which is herein incorporated by reference in their entirety). The therapeutic nanoparticle RNA vaccines, e.g., therapeutic nanoparticles comprising at least one RNA vaccine may be formulated using the methods described by Podobinski et al in U.S. Pat. No. 8,404,799, the contents of which are herein incorporated by reference in its entirety.

The RNA vaccines may be encapsulated in, linked to and/or associated with synthetic nanocarriers. Synthetic nanocarriers include, but are not limited to, those described in International Pub. Nos. WO2010005740, WO2010030763, WO201213501, WO2012149252, WO2012149255, WO2012149259, WO2012149265, WO2012149268, WO2012149282, WO2012149301, WO2012149393, WO2012149405, WO2012149411, WO2012149454 and WO2013019669, and US Pub. Nos. US20110262491, US20100104645, US20100087337 and US20120244222, each of which is herein incorporated by reference in their entirety. The synthetic nanocarriers may be formulated using methods known in the art and/or described herein. As a non-limiting example, the synthetic nanocarriers may be formulated by the methods described in International Pub Nos. WO2010005740, WO2010030763 and WO201213501 and US Pub. Nos. US20110262491, US20100104645, US20100087337 and US2012024422, each of which is herein incorporated by reference in their entirety. In another embodiment, the synthetic nanocarrier formulations may be lyophilized by methods described in International Pub. No. WO2011072218 and U.S. Pat. No. 8,211,473; the content of each of which is herein incorporated by reference in their entirety. In yet another embodiment, formulations of the present invention, including, but not limited to, synthetic nanocarriers, may be lyophilized or reconstituted by the methods described in US Patent Publication No. US20130230568, the contents of which are herein incorporated by reference in its entirety. In one embodiment, the synthetic nanocarriers may contain reactive groups to release the polynucleotides described herein (see International Pub. No. WO20120952552 and US Pub No. US20120171229, each of which is herein incorporated by reference in their entirety).

The synthetic nanocarriers may contain an immuno stimulatory agent to enhance the immune response from delivery of the synthetic nanocarrier. As a non-limiting example, the synthetic nanocarrier may comprise a Thl immuno stimulatory agent which may enhance a Thl-based response of the immune system (see International Pub No. WO2010123569 and US Pub. No. US20110223201, each of which is herein incorporated by reference in its entirety). The synthetic nanocarriers may be formulated for targeted release. In one embodiment, the synthetic nanocarrier is formulated to release the polynucleotides at a specified pH and/or after a desired time interval. As a non-limiting example, the synthetic nanoparticle may be formulated to release the RNA vaccines after 24 hours and/or at a pH of 4.5 (see International Pub. Nos. WO2010138193 and WO2010138194 and US Pub Nos. US20110020388 and US20110027217, each of which is herein incorporated by reference in their entireties).

The synthetic nanocarriers may be formulated for controlled and/or sustained release of the polynucleotides described herein. As a non-limiting example, the synthetic nanocarriers for sustained release may be formulated by methods known in the art, described herein and/or as described in International Pub No. WO2010138192 and US Pub No. 20100303850, each of which is herein incorporated by reference in their entirety. In one embodiment, the RNA vaccine may be formulated for controlled and/or sustained release wherein the formulation comprises at least one polymer that is a crystalline side chain (CYSC) polymer. CYSC polymers are described in U.S. Pat. No. 8,399,007, herein incorporated by reference in its entirety.

The synthetic nanocarrier may be formulated for use as a vaccine. In one embodiment, the synthetic nanocarrier may encapsulate at least one polynucleotide which encode at least one antigen. As a non-limiting example, the synthetic nanocarrier may include at least one antigen and an excipient for a vaccine dosage form (see International Pub No. WO2011150264 and US Pub No. US20110293723, each of which is herein incorporated by reference in their entirety). As another non-limiting example, a vaccine dosage form may include at least two synthetic nanocarriers with the same or different antigens and an excipient (see International Pub No. WO2011150249 and US Pub No. US20110293701, each of which is herein incorporated by reference in their entirety). The vaccine dosage form may be selected by methods described herein, known in the art and/or described in International Pub No. WO2011150258 and US Pub No. US20120027806, each of which is herein incorporated by reference in their entirety).

The synthetic nanocarrier may comprise at least one polynucleotide which encodes at least one adjuvant. As non-limiting example, the adjuvant may comprise dimethyldioctadecylammonium-bromide, dimethyldioctadecylammonium-chloride, dimethyldioctadecylammonium-phosphate or dimethyldioctadecylammonium-acetate (DDA) and an apolar fraction or part of said apolar fraction of a total lipid extract of a mycobacterium (See e.g, U.S. Pat. No. 8,241,610; herein incorporated by reference in its entirety). In another embodiment, the synthetic nanocarrier may comprise at least one polynucleotide and an adjuvant. As a non-limiting example, the synthetic nanocarrier comprising and adjuvant may be formulated by the methods described in International Pub No. WO2011150240 and US Pub No. US20110293700, each of which is herein incorporated by reference in its entirety. The synthetic nanocarrier may encapsulate at least one polynucleotide which encodes a peptide, fragment or region from a virus. As a non-limiting example, the synthetic nanocarrier may include, but is not limited to, the nanocarriers described in International Pub No. WO2012024621, WO201202629, WO2012024632 and US Pub No. US20120064110, US20120058153 and US20120058154, each of which is herein incorporated by reference in their entirety. T synthetic nanocarrier may be coupled to a polynucleotide which may be able to trigger a humoral and/or cytotoxic T lymphocyte (CTL) response (See e.g., International Publication No. WO2013019669, herein incorporated by reference in its entirety). In one embodiment, the RNA vaccine may be encapsulated in, linked to and/or associated with zwitterionic lipids. Non-limiting examples of zwitterionic lipids and methods of using zwitterionic lipids are described in US Patent Publication No. US20130216607, the contents of which are herein incorporated by reference in its entirety. In one aspect, the zwitterionic lipids may be used in the liposomes and lipid nanoparticles described herein. In one embodiment, the RNA vaccine may be formulated in colloid nanocarriers as described in US Patent Publication No. US20130197100, the contents of which are herein incorporated by reference in its entirety.

The nanoparticle may be optimized for oral administration. The nanoparticle may comprise at least one cationic biopolymer such as, but not limited to, chitosan or a derivative thereof. As a non-limiting example, the nanoparticle may be formulated by the methods described in U.S. Pub. No. 20120282343; herein incorporated by reference in its entirety. In some embodiments, LNPs comprise the lipid KL52 (an amino-lipid disclosed in U.S. Application Publication No. 2012/0295832 expressly incorporated herein by reference in its entirety). Activity and/or safety (as measured by examining one or more of ALT/AST, white blood cell count and cytokine induction) of LNP administration may be improved by incorporation of such lipids. LNPs comprising KL52 may be administered intravenously and/or in one or more doses. In some embodiments, administration of LNPs comprising KL52 results in equal or improved mRNA and/or protein expression as compared to LNPs comprising MC3.

In some embodiments, RNA vaccine may be delivered using smaller LNPs. Such particles may comprise a diameter from below 0.1 um up to 100 nm such as, but not limited to, less than 0.1 um, less than 1.0 um, less than 5 um, less than 10 um, less than 15 um, less than 20 um, less than 25 um, less than 30 um, less than 35 um, less than 40 um, less than 50 um, less than 55 um, less than 60 um, less than 65 um, less than 70 um, less than 75 um, less than 80 um, less than 85 um, less than 90 um, less than 95 um, less than 100 um, less than 125 um, less than 150 um, less than 175 um, less than 200 um, less than 225 um, less than 250 um, less than 275 um, less than 300 um, less than 325 um, less than 350 um, less than 375 um, less than 400 um, less than 425 um, less than 450 um, less than 475 um, less than 500 um, less than 525 um, less than 550 um, less than 575 um, less than 600 um, less than 625 um, less than 650 um, less than 675 um, less than 700 um, less than 725 um, less than 750 um, less than 775 um, less than 800 um, less than 825 um, less than 850 um, less than 875 um, less than 900 um, less than 925 um, less than 950 um, less than 975 um.

RNA vaccines may be delivered using smaller LNPs which may comprise a diameter from about 1 nm to about 100 nm, from about 1 nm to about 10 nm, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 50 nM, from about 20 to about 50 nm, from about 30 to about 50 nm, from about 40 to about 50 nm, from about 20 to about 60 nm, from about 30 to about 60 nm, from about 40 to about 60 nm, from about 20 to about 70 nm, from about 30 to about 70 nm, from about 40 to about 70 nm, from about 50 to about 70 nm, from about 60 to about 70 nm, from about 20 to about 80 nm, from about 30 to about 80 nm, from about 40 to about 80 nm, from about 50 to about 80 nm, from about 60 to about 80 nm, from about 20 to about 90 nm, from about 30 to about 90 nm, from about 40 to about 90 nm, from about 50 to about 90 nm, from about 60 to about 90 nm and/or from about 70 to about 90 nm.

Such LNPs may be synthesized using methods comprising microfluidic mixers. Exemplary microfluidic mixers may include, but are not limited to a slit interdigitial micromixer including, but not limited to those manufactured by Microinnova (Allerheiligen bei Wildon, Austria) and/or a staggered herringbone micromixer (SHM) (Zhigaltsev, I. V. et al., Bottom-up design and synthesis of limit size lipid nanoparticle systems with aqueous and triglyceride cores using millisecond microfluidic mixing have been published (Langmuir. 2012. 28:3633-40; Belliveau, N. M. et al., Microfluidic synthesis of highly potent limit-size lipid nanoparticles for in vivo delivery of siRNA. Molecular Therapy-Nucleic Acids. 2012. I: e37; Chen, D. et al., Rapid discovery of potent siRNA-containing lipid nanoparticles enabled by controlled microfluidic formulation. J Am Chem Soc. 2012. 134(16): 6948-51; each of which is herein incorporated by reference in its entirety). In some embodiments, methods of LNP generation comprising SHM, further comprise the mixing of at least two input streams wherein mixing occurs by microstructure-induced chaotic advection (MICA). According to this method, fluid streams flow through channels present in a herringbone pattern causing rotational flow and folding the fluids around each other. This method may also comprise a surface for fluid mixing wherein the surface changes orientations during fluid cycling. Methods of generating LNPs using SHM include those disclosed in U.S. Application Publication Nos. 2004/0262223 and 2012/0276209, each of which is expressly incorporated herein by reference in their entirety.

The RNA vaccine of the present invention may be formulated in lipid nanoparticles created using a micromixer such as, but not limited to, a Slit Interdigital Micro structured Mixer (SIMM-V2) or a Standard Slit Interdigital Micro Mixer (SSIMM) or Caterpillar (CPMM) or Impinging-jet (IJMM) from the Institut fiir Mikrotechnik Mainz GmbH, Mainz Germany). The RNA vaccines of the present invention may be formulated in lipid nanoparticles created using microfluidic technology (see Whitesides, George M. The Origins and the Future of Microfluidic s. Nature, 2006 442:368-373; and Abraham et al. Chaotic Mixer for Microchannels. Science, 2002 295:647-651; each of which is herein incorporated by reference in its entirety). As a non-limiting example, controlled microfluidic formulation includes a passive method for mixing streams of steady pressure-driven flows in micro channels at a low Reynolds number (See e.g., Abraham et al. Chaotic Mixer for Microchannels. Science, 2002 295:647-651; which is herein incorporated by reference in its entirety). The RNA vaccines of the present invention may be formulated in lipid nanoparticles created using a micromixer chip such as, but not limited to, those from Harvard Apparatus (Holliston, MA) or Dolomite Microfluidics (Royston, UK). A micromixer chip can be used for rapid mixing of two or more fluid streams with a split and recombine mechanism.

The RNA vaccines of the invention may be formulated for delivery using the drug encapsulating microspheres described in International Patent Publication No. WO2013063468 or U.S. Pat. No. 8,440,614, each of which is herein incorporated by reference in its entirety. The microspheres may comprise a compound of the formula (I), (II), (III), (IV), (V) or (VI) as described in International Patent Publication No. WO2013063468, the contents of which are herein incorporated by reference in its entirety. In another aspect, the amino acid, peptide, polypeptide, lipids (APPL) are useful in delivering the RNA vaccines of the invention to cells (see International Patent Publication No. WO2013063468, the contents of which is herein incorporated by reference in its entirety).

The RNA vaccines of the invention may be formulated in lipid nanoparticles having a diameter from about 10 to about 200 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 50 to about 150 nm, about 50 to about 200 nm, about 60 to about 70 nm, about 60 to about 80 nm, about 60 to about 90 nm, about 60 to about 100 nm, about 60 to about 150 nm, about 60 to about 200 nm, about 70 to about 80 nm, about 70 to about 90 nm, about 70 to about 100 nm, about 70 to about 150 nm, about 70 to about 200 nm, about 80 to about 90 nm, about 80 to about 100 nm, about 80 to about 150 nm, about 80 to about 200 nm, about 90 to about 100 nm, about 90 to about 150 nm, and/or about 90 to about 200 nm.

The lipid nanoparticles may have a diameter from about 10 to 500 nm. In one embodiment, the lipid nanoparticle may have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

The lipid nanoparticle may be a limit size lipid nanoparticle as described in International Patent Publication No. WO2013059922, the contents of which are herein incorporated by reference in its entirety. The limit size lipid nanoparticle may comprise a lipid bilayer surrounding an aqueous core or a hydrophobic core; where the lipid bilayer may comprise a phospholipid such as, but not limited to, diacylphosphatidylcholine, a diacylphosphatidylethanolamine, a ceramide, a sphingomyelin, a dihydrosphingomyelin, a cephalin, a cerebroside, a C8-C20 fatty acid diacylphophatidylcholine, and 1-palmitoyl-2-oleoyl phosphatidylcholine (POPC). In another aspect the limit size lipid nanoparticle may comprise a polyethylene glycol-lipid such as, but not limited to, DLPE-PEG, DMPE-PEG, DPPC-PEG and DSPE-PEG.

The RNA vaccines may be delivered, localized and/or concentrated in a specific location using the delivery methods described in International Patent Publication No. WO2013063530, the contents of which are herein incorporated by reference in its entirety. As a non-limiting example, a subject may be administered an empty polymeric particle prior to, simultaneously with or after delivering the RNA vaccines to the subject. The empty polymeric particle undergoes a change in volume once in contact with the subject and becomes lodged, embedded, immobilized or entrapped at a specific location in the subject.

The RNA vaccines may be formulated in an active substance release system (See e.g., US Patent Publication No. US20130102545, the contents of which is herein incorporated by reference in its entirety). The active substance release system may comprise 1) at least one nanoparticle bonded to an oligonucleotide inhibitor strand which is hybridized with a catalytically active nucleic acid and 2) a compound bonded to at least one substrate molecule bonded to a therapeutically active substance (e.g., polynucleotides described herein), where the therapeutically active substance is released by the cleavage of the substrate molecule by the catalytically active nucleic acid.

The RNA vaccines may be formulated in a nanoparticle comprising an inner core comprising a non-cellular material and an outer surface comprising a cellular membrane. The cellular membrane may be derived from a cell or a membrane derived from a virus. As a non-limiting example, the nanoparticle may be made by the methods described in International Patent Publication No. WO2013052167, herein incorporated by reference in its entirety. As another non-limiting example, the nanoparticle described in International Patent Publication No. WO2013052167, herein incorporated by reference in its entirety, may be used to deliver the RNA vaccines described herein.

The RNA vaccines may be formulated in porous nanoparticle-supported lipid bilayers (protocells). Protocells are described in International Patent Publication No. WO2013056132, the contents of which are herein incorporated by reference in its entirety.

The RNA vaccines described herein may be formulated in polymeric nanoparticles as described in or made by the methods described in U.S. Pat. Nos. 8,420,123 and 8,518,963 and European Patent No. EP2073848B 1, the contents of each of which are herein incorporated by reference in their entirety. As a non-limiting example, the polymeric nanoparticle may have a high glass transition temperature such as the nanoparticles described in or nanoparticles made by the methods described in U.S. Pat. No. 8,518,963, the contents of which are herein incorporated by reference in its entirety. As another non-limiting example, the polymer nanoparticle for oral and parenteral formulations may be made by the methods described in European Patent No. EP2073848B 1, the contents of which are herein incorporated by reference in its entirety.

The RNA vaccines described herein may be formulated in nanoparticles used in imaging. The nanoparticles may be liposome nanoparticles such as those described in US Patent Publication No US20130129636, herein incorporated by reference in its entirety. As a non-limiting example, the liposome may comprise gadolinium (III) 2-{4,7-bis-carboxymethyl-10-[(N,N-distearylamidomethyl-N′-amido-methyl]-1,4,7,10-tetra-azacyclododec-1-yl}-acetic acid and a neutral, fully saturated phospholipid component (see e.g., US Patent Publication No US20130129636, the contents of which is herein incorporated by reference in its entirety).

The nanoparticles which may be used in the present invention may be formed by the methods described in U.S. Patent Application No. US20130130348, the contents of which is herein incorporated by reference in its entirety. The nanoparticles of the present invention may further include nutrients such as, but not limited to, those which deficiencies can lead to health hazards from anemia to neural tube defects (see e.g, the nanoparticles described in International Patent Publication No WO2013072929, the contents of which is herein incorporated by reference in its entirety). As a non-limiting example, the nutrient may be iron in the form of ferrous, ferric salts or elemental iron, iodine, folic acid, vitamins or micronutrients.

RNA vaccines of the present invention may be formulated in a swellable nanoparticle. The swellable nanoparticle may be, but is not limited to, those described in U.S. Pat. No. 8,440,231, the contents of which is herein incorporated by reference in its entirety. As a non-limiting embodiment, the swellable nanoparticle may be used for delivery of the RNA vaccines of the present invention to the pulmonary system (see e.g., U.S. Pat. No. 8,440,231, the contents of which is herein incorporated by reference in its entirety).

RNA vaccines of the present invention may be formulated in polyanhydride nanoparticles such as, but not limited to, those described in U.S. Pat. No. 8,449,916, the contents of which is herein incorporated by reference in its entirety.

The nanoparticles and microparticles of the present invention may be geometrically engineered to modulate macrophage and/or the immune response. In one aspect, the geometrically engineered particles may have varied shapes, sizes and/or surface charges in order to incorporated the polynucleotides of the present invention for targeted delivery such as, but not limited to, pulmonary delivery (see e.g., International Publication No WO2013082111, the contents of which is herein incorporated by reference in its entirety). Other physical features the geometrically engineering particles may have include, but are not limited to, fenestrations, angled arms, asymmetry and surface roughness, charge which can alter the interactions with cells and tissues. As a non-limiting example, nanoparticles of the present invention may be made by the methods described in International Publication No WO2013082111, the contents of which is herein incorporated by reference in its entirety.

Nanoparticles of the present invention may be water soluble nanoparticles such as, but not limited to, those described in International Publication No. WO2013090601, the contents of which is herein incorporated by reference in its entirety. The nanoparticles may be inorganic nanoparticles which have a compact and zwitterionic ligand in order to exhibit good water solubility. The nanoparticles may also have small hydrodynamic diameters (HD), stability with respect to time, pH, and salinity and a low level of non-specific protein binding. In one embodiment the nanoparticles of the present invention may be developed by the methods described in US Patent Publication No. US20130172406, the contents of which are herein incorporated by reference in its entirety. Nanoparticles of the present invention may be stealth nanoparticles or target-specific stealth nanoparticles such as, but not limited to, those described in US Patent Publication No. US20130172406; the contents of which is herein incorporated by reference in its entirety. The nanoparticles of the present invention may be made by the methods described in US Patent Publication No. US20130172406, the contents of which are herein incorporated by reference in its entirety.

The stealth or target-specific stealth nanoparticles may comprise a polymeric matrix. The polymeric matrix may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polyesters, polyanhydrides, polyethers, polyurethanes, polymethacrylates, polyacrylates, polycyanoacrylates or combinations thereof. The nanoparticle may be a nanoparticle-nucleic acid hybrid structure having a high density nucleic acid layer. As a non-limiting example, the nanoparticle-nucleic acid hybrid structure may made by the methods described in US Patent Publication No. US20130171646, the contents of which are herein incorporated by reference in its entirety. The nanoparticle may comprise a nucleic acid such as, but not limited to, polynucleotides described herein and/or known in the art.

Nanoparticles may be embedded in in the core a nanostructure or coated with a low density porous 3-D structure or coating which is capable of carrying or associating with at least one pay load within or on the surface of the nanostructure. Non-limiting examples of the nanostructures comprising at least one nanoparticle are described in International Patent Publication No. WO2013123523, the contents of which are herein incorporated by reference in its entirety.

Other formulations include those which are described in US20170136121 A1, U.S. Pat. No. 9,221,891 B2, EP2971033 B1 and US20160331828 A1 which are herein incorporated by reference in their entirety.

Also provided is a method of producing a an mRNA encoding at least one immunogenic peptide fragment, the method comprising:
(a) binding a first polynucleotide comprising an open reading frame encoding the immunogenic peptide fragment and a second polynucleotide comprising a 5′-UTR to a polynucleotide conjugated to a solid support;
(b) ligating the 3′-terminus of the second polynucleotide to the 5′-terminus of the first polynucleotide under suitable conditions, wherein the suitable conditions comprise a DNA Ligase, thereby producing a first ligation product;
(c) ligating the 5′ terminus of a third polynucleotide comprising a 3′-UTR to the 3′-terminus of the first ligation product under suitable conditions, wherein the suitable conditions comprise an RNA Ligase, thereby producing a second ligation product; and
(d) releasing the second ligation product from the solid support, thereby producing an mRNA encoding the immunogenic peptide fragment.

Also provided is a kit for preparing an mRNA cancer vaccine. The kit may have one or more containers housing one or more polynucleotides comprising a 5′-ORF, one or more polynucleotides comprising a 3′-ORF, one or more polynucleotides comprising a poly(A) tail, a ligase enzyme, and instructions for ligating one or more polynucleotides comprising an ORF encoding a patient specific epitope to the one or more polynucleotides comprising the a 5′-ORF, 3′-ORF, and poly(A) tail.

### SEQUENCE INFORMATION

TABLE X
sequences for human immune checkpoints

| SEQ | Reference no. | |
|---|---|---|
| NO. | or name | Sequence |
| (a) | Checkpoint = Interaction of IDO and substrate | |
| | Preferred polypeptide component = IDO | |
| 1 | Full length | MAHAMENSWTISKEYHIDEEVGFALPNPQENLPDFYNDWMFIAKHLPDLIESGQL |
| | IDO | RERVEKLNMLSIDHLTDHKSQRLARLVLGCITMAYVWGKGHGDVRKVLPRNIAVP |
| | | YCQLSKKLELPPILVYADCVLANWKKKDPNKPLTYENMDVLFSFRDGDCSKGFFL |
| | | VSLLVEIAAASAIKVIPTVFKAMQMQERDTLLKALLEIASCLEKALQVFHQIHDH |
| | | VNPKAFFSVLRIYLSGWKGNPQLSDGLVYEGFWEDPKEFAGGSAGQSSVFQCFDV |
| | | LLGIQQTAGGGHAAQFLQDMRRYMPPAHRNFLCSLESNPSVREFVLSKGDAGLRE |
| | | AYDACVKALVSLRSYHLQIVTKYILIPASQQPKENKTSEDPSKLEAKGTGGTDLM |
| | | NFLKTVRSTTEKSLLKEG |
| 2 | IO102 | DTLLKALLEIASCLEKALQVF |
| 3 | IO101 | ALLEIASCL |
| 4 | | QLRERVEKL |
| 5 | | FLVSLLVEI |
| 6 | | TLLKALLEI |
| 7 | | FIAKHLPDL |
| 8 | | VLSKGDAGL |
| 9 | | DLMNFLKTV |
| 10 | | VLLGIQQTA |
| 11 | | KVLPRNIAV |
| 12 | | KLNMLSIDHL |
| 13 | | SLRSYHLQIV |
| (b) | Checkpoint = Interaction of PD1 and PD-L1 | |
| | Preferred polypeptide component = PD-L1 | |
| 14 | Full length | MRIFAVFIFMTYWHLLNAFTVTVPKDLYVVEYGSNMTIECKFPVEKQLDLAALIV |
| | PD-L1 | YWEMEDKNIIQFVHGEEDLKVQHSSYRQRARLLKDQLSIGNAALQITDVKLQDAG |
| | | VYRCMISYGGADYKRITVKVNAPYNKINQRILVVDPVTSEHELTCQAEGYPKAEV |
| | | IWTSSDHQVLSGKTTTTNSKREEKLFNVTSTLRINTTTNEIFYCTFRRLDPEENH |
| | | TAELVIPELPLAHPPNERTHLVILGAILLCLGVALTFIFRLRKGRMMDVKKCGIQ |
| | | DTNSKKQSDTHLEET |
| 15 | PDlong1 | FMTYWHLLNAFTVTVPKDL |
| | IO103 | |
| 16 | PDlong2.1 | RTHLVILGAILLCLGVALTFIFRLRKGR |
| | IO104.1 | |
| 17 | PDlong2 / | VILGAILLCLGVALTFIFRLRKG |
| | IO104 | |
| 18 | PDL101 | LLNAFTVTV |
| 19 | PDL116 | FIFMTYWHLL |
| 20 | PDL1₁₅₋₂₃ | LLNAFTVTV |
| 21 | PDL102 | ILLCLGVAL |
| 22 | PDL103 | ILGAILLCL |
| 23 | PDL109 | HLVILGAIL |
| 24 | PDL111 | CLGVALTFI |
| 25 | PDL114 | VILGAILLCL |
| 26 | | VILGAILLCLGVALTFI |
| 27 | | VILGAILLCLGVALTFIF |
| 28 | | VILGAILLCLGVALTFIFR |
| 29 | | VILGAILLCLGVALTFIFRL |
| 30 | | VILGAILLCLGVALTFIFRLR |
| 31 | | VILGAILLCLGVALTFIFRLRK |
| 32 | | VILGAILLCLGVALTFIFRLRKGR |
| 33 | | VILGAILLCLGVALTFIFRLRKGRM |
| 34 | | VILGAILLCLGVALTFIFRLRKGRMM |
| 35 | | VILGAILLCLGVALTFIFRLRKGRMMD |
| 36 | | LVILGAILLCLGVALTFI |
| 37 | | LVILGAILLCLGVALTFIF |
| 38 | | LVILGAILLCLGVALTFIFR |
| 39 | | LVILGAILLCLGVALTFIFRL |
| 40 | | LVILGAILLCLGVALTFIFRLR |
| 41 | | LVILGAILLCLGVALTFIFRLRK |
| 42 | | LVILGAILLCLGVALTFIFRLRKG |
| 43 | | LVILGAILLCLGVALTFIFRLRKGR |
| 44 | | LVILGAILLCLGVALTFIFRLRKGRM |
| 45 | | LVILGAILLCLGVALTFIFRLRKGRMM |
| 46 | | LVILGAILLCLGVALTFIFRLRKGRMMD |
| 47 | | HLVILGAILLCLGVALTFI |
| 48 | | HLVILGAILLCLGVALTFIF |
| 49 | | HLVILGAILLCLGVALTFIFR |
| 50 | | HLVILGAILLCLGVALTFIFRL |
| 51 | | HLVILGAILLCLGVALTFIFRLR |
| 52 | | HLVILGAILLCLGVALTFIFRLRK |
| 53 | | HLVILGAILLCLGVALTFIFRLRKG |
| 54 | | HLVILGAILLCLGVALTFIFRLRKGR |
| 55 | | HLVILGAILLCLGVALTFIFRLRKGRM |
| 56 | | HLVILGAILLCLGVALTFIFRLRKGRMM |
| 57 | | HLVILGAILLCLGVALTFIFRLRKGRMMD |
| 58 | | THLVILGAILLCLGVALTFI |
| 59 | | THLVILGAILLCLGVALTFIF |
| 60 | | THLVILGAILLCLGVALTFIFR |
| 61 | | THLVILGAILLCLGVALTFIFRL |
| 62 | | THLVILGAILLCLGVALTFIFRLR |
| 63 | | THLVILGAILLCLGVALTFIFRLRK |
| 64 | | THLVILGAILLCLGVALTFIFRLRKG |
| 65 | | THLVILGAILLCLGVALTFIFRLRKGR |
| 66 | | THLVILGAILLCLGVALTFIFRLRKGRM |
| 67 | | THLVILGAILLCLGVALTFIFRLRKGRMM |
| 68 | | THLVILGAILLCLGVALTFIFRLRKGRMMD |
| 69 | | RTHLVILGAILLCLGVALTFI |
| 70 | | RTHLVILGAILLCLGVALTFIF |
| 71 | | RTHLVILGAILLCLGVALTFIFR |
| 72 | | RTHLVILGAILLCLGVALTFIFRL |
| 73 | | RTHLVILGAILLCLGVALTFIFRLR |
| 74 | | RTHLVILGAILLCLGVALTFIFRLRK |
| 75 | | RTHLVILGAILLCLGVALTFIFRLRKG |
| 76 | | RTHLVILGAILLCLGVALTFIFRLRKGRM |
| 77 | | RTHLVILGAILLCLGVALTFIFRLRKGRMM |
| 78 | | RTHLVILGAILLCLGVALTFIFRLRKGRMMD |
| 79 | | ERTHLVILGAILLCLGVALTFI |
| 80 | | ERTHLVILGAILLCLGVALTFIF |
| 81 | | ERTHLVILGAILLCLGVALTFIFR |
| 82 | | ERTHLVILGAILLCLGVALTFIFRL |
| 83 | | ERTHLVILGAILLCLGVALTFIFRLR |
| 84 | | ERTHLVILGAILLCLGVALTFIFRLRK |
| 85 | | ERTHLVILGAILLCLGVALTFIFRLRKG |
| 86 | | ERTHLVILGAILLCLGVALTFIFRLRKGR |
| 87 | | ERTHLVILGAILLCLGVALTFIFRLRKGRM |
| 88 | | ERTHLVILGAILLCLGVALTFIFRLRKGRMM |
| 89 | | ERTHLVILGAILLCLGVALTFIFRLRKGRMMD |
| 90 | | NERTHLVILGAILLCLGVALTFI |
| 91 | | NERTHLVILGAILLCLGVALTFIF |
| 92 | | NERTHLVILGAILLCLGVALTFIFR |
| 93 | | NERTHLVILGAILLCLGVALTFIFRL |
| 94 | | NERTHLVILGAILLCLGVALTFIFRLR |
| 95 | | NERTHLVILGAILLCLGVALTFIFRLRK |
| 96 | | NERTHLVILGAILLCLGVALTFIFRLRKG |
| 97 | | NERTHLVILGAILLCLGVALTFIFRLRKGR |
| 98 | | NERTHLVILGAILLCLGVALTFIFRLRKGRM |
| 99 | | NERTHLVILGAILLCLGVALTFIFRLRKGRMM |
| 100 | | NERTHLVILGAILLCLGVALTFIFRLRKGRMMD |
| (b) | Checkpoint = Interaction of PD1 and PD-L2 | |
| | Preferred polypeptide component = PD-L2 | |
| 101 | Full length | MIFLLLMLSLELQLHQIAALFTVTVPKELYIIEHGSNVTLECNFDTGSHVNLGAI |
| | PD-L2 | TASLQKVENDTSPHRERATLLEEQLPLGKASFHIPQVQVRDEGQYQCIIIYGVAW |
| | | DYKYLTLKVKASYRKINTHILKVPETDEVELTCQATGYPLAEVSWPNVSVPANTS |
| | | HSRTPEGLYQVTSVL |
| | | RLKPPPGRNFSCVFWNTHVRELTLASIDLQSQMEPRTHPTWLLHIFIPFCIIAFI |
| | | FIATVIALRKQLCQKLYSSKDTTKRPVTTTKREVNSAI |
| 102 | PDL2long1 | SLELQLHQIAALFTVTVPKEL |
| 103 | PDL2long2 | MIFLLLMLSLELQLHQIAALFTVTV |
| 104 | PDL2 201 | LLLMLSLEL |
| (c) | Checkpoint = Interaction of Arginasel and substrate | |
| | Preferred polypeptide component = Arginasel | |
| 105 | Full length | MSAKSRTIGI IGAPFSKGQP RGGVEEGPTV LRKAGLLEKL KEQECDVKDY |
| | Arginase1 | GDLPFADIPN DSPFQIVKNP RSVGKASEQL AGKVAEVKKN GRISLVLGGD |
| | | HSLAIGSISG HARVHPDLGV IWVDAHTDIN TPLTTTSGNL HGQPVSFLLK |
| | | ELKGKIPDVP GFSWVTPCIS AKDIVYIGLR DVDPGEHYIL KTLGIKYFSM |
| | | TEVDRLGIGK VMEETLSYLL GRKKRPIHLS FDVDGLDPSF TPATGTPVVG |
| | | GLTYREGLYI TEEIYKTGLL SGLDIMEVNP SLGKTPEEVT RTVNTAVAIT |
| | | LACFGLAREG NHKPIDYLNP PK |
| 106 | Arglong2 / | ISAKDIVYIGLRDVDPGEHYILKTLGIKYFSMTEVDRL |
| | IO112 | |
| 107 | Arglong3 | ISAKDIVYIGLRDVDPGEHYILKTLGIKYFSM |
| 108 | Arglong | ISAKDIVYIGLRDVDPGEHYILKTLGIKYFSMTEVDRLGIGK |
| 109 | Arg1-18 | AKDIVYIGLRDVDPGEHYIL |
| 110 | Arg1-19 | DVDPGEHYILKTLGIKYFSM |
| 111 | Arg1-20 | KTLGIKYFSMTEVDRLGIGK |
| 112 | Hotspot1 | GFSWVTPCISAKDIVYIGLRDVDPGEHYILKTLGIKYFSMTEVDRLGIGK |
| 113 | Arg1 | QLAGKVAEV |
| 114 | Arg2 | LVLGGDHSL |
| 115 | Arg3 | LLKELKGKI |
| 116 | Arg4 | ELKGKIPDV |
| 117 | Arg5 | VMEETLSYL |
| 118 | Arg6 | HLSFDVDGL |
| 119 | Arg7 | SLGKTPEEV |
| 120 | Arg8 | IVYIGLRDV |
| 121 | Arg9 | GLLSGLDIM |
| 122 | Arg10 | DIMEVNPSL |
| 123 | Arg11 | LLSGLDIMEV |
| 124 | Arg12 | NLHGQPVSFL |
| 125 | Arg13 | SLVLGGDHSL |
| 126 | Arg14 | YLLGRKKRPI |
| 127 | Arg15 | VLGGDHSLAI |
| 128 | Arg16 | FLLKELKGKI |
| 129 | Arg1-1 | MSAKSRTIGIIGAPFSKGQP |
| 130 | Arg1-2 | IGAPFSKGQPRGGVEEGPTV |
| 131 | Arg1-3 | RGGVEEGPTVLRKAGLLEKL |
| 132 | Arg1-4 | LRKAGLLEKLKEQECDVKDY |
| 133 | Arg1-5 | KEQECDVKDYGDLPFADIPN |
| 134 | Arg1-6 | GDLPFADIPNDSPFQIVKNP |
| 135 | Arg1-7 | DSPFQIVKNPRSVGKASEQL |
| 136 | Arg1-8 | RSVGKASEQLAGKVAEVKKN |
| 137 | Arg1-9 | AGKVAEVKKNGRISLVLGGD |
| 138 | Arg1-10 | GRISLVLGGDHSLAIGSISG |
| 139 | Arg1-11 | HSLAIGSISGHARVHPDLGV |
| 140 | Arg1-12 | HARVHPDLGVIWVDAHTDIN |
| 141 | Arg1-13 | IWVDAHTDINTPLTTTSGNL |
| 142 | Arg1-14 | TPLTTTSGNLHGQPVSFLLK |
| 143 | Arg1-15 | HGQPVSFLLKELKGKIPDVP |
| 144 | Arg1-16 | ELKGKIPDVPGFSWVTPCIS |
| 145 | Arg1-17 | GFSWVTPCISAKDIVYIGLR |
| 146 | Arg1-21 | TEVDRLGIGKVMEETLSYLL |
| 147 | Arg1-22 | VMEETLSYLLGRKKRPIHLS |
| 148 | Arg1-23 | GRKKRPIHLSFDVDGLDPSF |
| 149 | Arg1-24 | FDVDGLDPSFTPATGTPVVG |
| 150 | Arg1-25 | TPATGTPVVGGLTYREGLYI |
| 151 | Arg1-26 | GLTYREGLYITEEIYKTGLL |
| 152 | Arg1-27 | TEEIYKTGLLSGLDIMEVNP |
| 153 | Arg1-28 | SGLDIMEVNPSLGKTPEEVT |
| 154 | Arg1-29 | SLGKTPEEVTRTVNTAVAIT |
| 155 | Arg1-30 | RTVNTAVAITLACFGLAREG |
| 156 | Arg1-31 | LACFGLAREGNHKPIDYLNPPK |
| 157 | | GLYITEEIYKTGLLSGLDIM |
| 158 | | GFSWVTPCISAKDIVYIGLRDVDPGEHYIL |
| (c) | Checkpoint = Interaction of Arginase2 and substrate | |
| | Preferred polypeptide component = Arginase2 | |
| 159 | Full length | MSLRGSLSRL LQTRVHSILK KSVHSVAVIG APFSQGQKRK GVEHGPAAIR |
| | Arginase2 | EAGLMKRLSS LGCHLKDFGD LSFTPVPKDD LYNNLIVNPR SVGLANQELA |
| | | EVVSRAVSDG YSCVTLGGDH SLAIGTISGH ARHCPDLCVV WVDAHADINT |
| | | PLTTSSGNLH GQPVSFLLRE LQDKVPQLPG FSWIKPCISS ASIVYIGLRD |
| | | VDPPEHFILK NYDIQYFSMR DIDRLGIQKV MERTFDLLIG KRQRPIHLSF |
| | | DIDAFDPTLA PATGTPVVGG LTYREGMYIA EEIHNTGLLS ALDLVEVNPQ |
| | | LATSEEEAKT TANLAVDVIA SSFGQTREGG HIVYDQLPTP SSPDESENQA |
| | | RVRI |
| 160 | A2L2 | SLRGSLSRLLQTRVHSILKKSVHSVAVIGAPFS |
| 161 | A2L3 | SLSRLLQTRVHSILKKSVHSVAVIGAPFS |
| 162 | A2L1 | SLRGSLSRLLQTRVHSILKKSVHSVAVIGA |
| 163 | A2S05 | NLIVNPRSV |
| 164 | A2S14 | GLLSALDLV |
| 165 | A2S15 | LLSALDLVEV |
| 166 | A2S01 | SLRGSLSRL |
| 167 | A2S02 | LLQTRVHSI |
| 168 | A2S03 | ILKKSVHSV |
| 169 | A2S04 | GLMKRLSSL |
| 170 | A2S06 | LANQELAEV |
| 171 | A2S07 | VTLGGDHSL |
| 172 | A2S08 | FLLRELQDKV |
| 173 | A2S09 | LLRELQDKV |
| 174 | A2S10 | ELQDKVPQL |
| 175 | A2S11 | IVYIGLRDV |
| 176 | A2S12 | KVMERTFDL |
| 177 | A2S13 | YIAEEIHNT |
| 178 | ARG1 65-73 | QIVKNPRSV |
| 179 | N terminus | MSLRGSLSRLLQTRVHSILKKSVHSVAVIGAPFSQGQKRKGVEHGPAAIREAGLM |
| | incl signal | K |
| 180 | Arg2 181-209 | FSWIKPCISSASIVYIGLRDVDPPEHFIL |
| 181 | Arg2 178-209 | LPGFSWIKPCISSASIVYIGLRDVDPPEHFIL |
| 182 | Hotspot2 | GFSWIKPCISSASIVYIGLRDVDPPEHFILKNYDIQYFSMRDIDRLGIQK |
| 183 | ARG2 | MSLRGSLSRLLQTRVHSILK |
| 184 | ARG2 1 | LQTRVHSILKKSVHSVAVIG |
| 185 | ARG2 2 | KSVHSVAVIGAPFSQGQKRK |
| 186 | ARG2 3 | APFSQGQKRKGVEHGPAAIR |
| 187 | ARG2 4 | GVEHGPAAIREAGLMKRLSS |
| 188 | ARG2 5 | EAGLMKRLSSLGCHLKDFGD |
| 189 | ARG2 6 | LGCHLKDFGDLSFTPVPKDD |
| 190 | ARG2 7 | LSFTPVPKDDLYNNLIVNPR |
| 191 | ARG2 8 | LYNNLIVNPRSVGLANQELA |
| 192 | ARG2 9 | SVGLANQELAEVVSRAVSDG |
| 193 | ARG2 10 | EVVSRAVSDGYSCVTLGGDH |
| 194 | ARG2 11 | YSCVTLGGDHSLAIGTISGH |
| 195 | ARG2 12 | SLAIGTISGHARHCPDLCVV |
| 196 | ARG2 13 | ARHCPDLCVVWVDAHADINT |
| 197 | ARG2 14 | WVDAHADINTPLTTSSGNLH |
| 198 | ARG2 15 | PLTTSSGNLHGQPVSFLLRE |
| 199 | ARG2 16 | GQPVSFLLRELQDKVPQLPG |
| 200 | ARG2 17 | LQDKVPQLPGFSWIKPCISS |
| 201 | ARG2 18 | FSWIKPCISSASIVYIGLRD |
| 202 | ARG2 19 | ASIVYIGLRDVDPPEHFILK |
| 203 | ARG2 20 | VDPPEHFILKNYDIQYFSMR |
| 204 | ARG2 21 | NYDIQYFSMRDIDRLGIQKV |
| 205 | ARG2 22 | DIDRLGIQKVMERTFDLLIG |
| 206 | ARG2 23 | MERTFDLLIGKRQRPIHLSF |
| 207 | ARG2 24 | KRQRPIHLSFDIDAFDPTLA |
| 208 | ARG2 25 | DIDAFDPTLAPATGTPVVGG |
| 209 | ARG2 26 | PATGTPVVGGLTYREGMYIA |
| 210 | ARG2 27 | LTYREGMYIAEEIHNTGLLS |
| 211 | ARG2 28 | EEIHNTGLLSALDLVEVNPQ |
| 212 | ARG2 29 | ALDLVEVNPQLATSEEEAKT |
| 213 | ARG2 30 | LATSEEEAKTTANLAVDVIA |
| 214 | ARG2 31 | TANLAVDVIASSFGQTREGG |
| 215 | ARG2 32 | SSFGQTREGGHIVYDQLPTP |
| 216 | ARG2 33 | HIVYDQLPTPSSPDESENQARVRI |
| 217 | ARG2-E17 | GFSWIKPCISSASIVYIGLR |
| 218 | ARG2-E18 | SASIVYIGLRDVDPPEHFIL |
| 219 | ARG2-E19 | DVDPPEHFILKNYDIQYFSM |
| 220 | ARG2-E20 | KNYDIQYFSMRDIDRLGIQK |
| (d) | Checkpoint = Interaction of TDO and substrate | |
| | Preferred polypeptide component = TDO | |
| 221 | Full length | MSGCPFLGNNFGYTFKKLPVEGSEEDKSQTGVNRASKGGLIYGNYLHLEKVLNAQ |
| | TDO | ELQSETKGNKIHDEHLFIITHQAYELWFKQILWELDSVREIFQNGHVRDERNMLK |
| | | VVSRMHRVSVILKLLVQQFSILETMTALDFNDFREYLSPASGFQSLQFRLLENKI |
| | | GVLQNMRVPYNRRHYRDNFKGEENELLLKSEQEKTLLELVEAWLERTPGLEPHGF |
| | | NFWGKLEKNITRGLEEEFIRIQAKEESEEKEEQVAEFQKQKEVLLSLFDEKRHEH |
| | | LLSKGERRLSYRALQGALMIYFYREEPRFQVPFQLLTSLMDIDSLMTKWRYNHVC |
| | | MVHRMLGSKAGTGGSSGYHYLRSTVSDRYKVFVDLFNLSTYLIPRHWIPKMNPTI |
| | | HKFLYTAEYCDSSYFSSDESD |
| 222 | TDO1 | RLLENKIGVL |
| 223 | TDO2 | TLLELVEAWL |
| 224 | TDO3 | FIITHQAYEL |
| 225 | TDO4 | LIYGNYLHL |
| 226 | TDO4 | KLLVQQFSIL |
| 227 | TDO5 | KIHDEHLFII |
| 228 | TDO6 | LLKSEQEKTL |
| 229 | TDO6 | QLLTSLMDI |
| 230 | TDO7 | QILWELDSV |
| 231 | TDO8 | SILETMTAL |
| 232 | TDO9 | LLSKGERRL |
| 233 | TDO10 | DLFNLSTYL |
| 234 | TDO11 | KLEKNITRGL |
| 235 | TDO12 | LIPRHWIPKM |
| 236 | TDO13 | KMNPTIHKFL |
| 237 | TDO14 | VSVILKLLVQQFSILETMTA |
| 238 | TDO15 | RFQVPFQLLTSLMDIDSLMT |
| (e) | Checkpoint = The interaction between TGFb1 and its receptors | |
| | Preferred polypeptide component = TGFb1 | |
| 239 | Full length | MPPSGLRLLL LLLPLLWLLV LTPGRPAAGL STCKTIDMEL VKRKRIEAIR |
| | TGFb1 | GQILSKLRLA SPPSQGEVPP GPLPEAVLAL YNSTRDRVAG ESAEPEPEPE |
| | | ADYYAKEVTR VLMVETHNEI YDKFKQSTHS IYMFFNTSEL REAVPEPVLL |
| | | SRAELRLLRL KLKVEQHVEL YQKYSNNSWR YLSNRLLAPS DSPEWLSFDV |
| | | TGVVRQWLSR GGEIEGFRLS AHCSCDSRDN TLQVDINGFT TGRRGDLATI |
| | | HGMNRPFLLL MATPLERAQH LQSSRHRRAL DTNYCFSSTE KNCCVRQLYI |
| | | DFRKDLGWKW IHEPKGYHAN FCLGPCPYIW SLDTQYSKVL ALYNQHNPGA |
| | | SAAPCCVPQA LEPLPIVYYV GRKPKVEQLS NMIVRSCKCS |
| 240 | TGFb1 signal | MPPSGLRLLLLLLPLLWLLVLTPGRPAAG |
| | peptide (SP) | |
| 241 | TGFb1 LAP | LSTCKTIDMELVKRKRIEAIRGQILSKLRLASPPSQGEVPPGPLPEAVL |
| | | ALYNSTRDRVAGESAEPEPEPEADYYAKEVTRVLMVETHNEIYDKFKQS |
| | | THSIYMFFNTSELREAVPEPVLLSRAELRLLRLKLKVEQHVELYQKYSN |
| | | NSWRYLSNRLLAPSDSPEWLSFDVTGVVRQWLSRGGEIEGFRLSAHCSC |
| | | DSRDNTLQVDINGFTTGRRGDLATIHGMNRPFLLLMATPLERAQHLQSS |
| | | RHRR |
| 242 | Mature TGFb1 | ALDTNYCFSSTEKNCCVRQLYIDFRKDLGWKWIHEPKGYHANFCLGPCP |
| | | YIWSLDTQYSKVLALYNQHNPGASAAPCCVPQALEPLPIVYYVGRKPKV |
| | | EQLSNMIVRSCKCS |
| 243 | TGFb-15 | REAVPEPVLLSRAELRLLRL |
| 244 | TGFb-short | VLLSRAELRL |
| 245 | TGFb-15.1 | NTSELREAVPEPVLLSRAELRLLRL |
| 246 | TGFb-15.2 | REAVPEPVLLSRAELRLLRLKLKVE |
| 247 | TGFb-15.3 | NTSELREAVPEPVLLSRAELRLLRLKLKVE |
| 248 | TGFb-01 | MPPSGLRLLLLLLPLLWLLV |
| 249 | TGFb-A2-01 | LLLLLPLLWL |
| 250 | TGFb-02 | LLLPLLWLLVLTPGRPAAGL |
| 251 | TGFb-02.1 | LLLPLLWLLVLTPGRPAAGLSTCKT |
| 252 | TGFb-02.2 | LRLLLLLLPLLWLLVLTPGRPAAGL |
| 253 | TGFb-02.3 | LRLLLLLLPLLWLLVLTPGRPAAGLSTCKT |
| 254 | TGFb-03 | LTPGRPAAGLSTCKTIDMEL |
| 255 | TGFb-04 | STCKTIDMELVKRKRIEAIR |
| 256 | TGFb-05 | VKRKRIEAIRGQILSKLRLA |
| 257 | TGFb-05.1 | IDMELVKRKRIEAIRGQILSKLRLA |
| 258 | TGFb-05.2 | VKRKRIEAIRGQILSKLRLASPPSQ |
| 259 | TGFb-05.3 | IDMELVKRKRIEAIRGQILSKLRLASPPSQ |
| 260 | TGFb-06 | GQILSKLRLASPPSQGEVPP |
| 261 | TGFb-07 | SPPSQGEVPPGPLPEAVLAL |
| 262 | TGFb-08 | GPLPEAVLALYNSTRDRVAG |
| 263 | TGFb-09 | YNSTRDRVAGESAEPEPEPE |
| 264 | TGFb-10 | ESAEPEPEPEADYYAKEVTR |
| 265 | TGFb-11 | ADYYAKEVTRVLMVETHNEI |
| 266 | TGFb-12 | VLMVETHNEIYDKFKQSTHS |
| 267 | TGFb-13 | YDKFKQSTHSIYMFFNTSEL |
| 268 | TGFb-13.1 | THNEIYDKFKQSTHSIYMFFNTSEL |
| 269 | TGFb-13.2 | YDKFKQSTHSIYMFFNTSELREAVP |
| 270 | TGFb-13.3 | THNEIYDKFKQSTHSIYMFFNTSELREAVP |
| 271 | TGFb-14 | IYMFFNTSELREAVPEPVLL |

TABLE 2
predicted mRNA ORF sequences for encoding fragments of human
immune checkpoints

| SEQ | | |
|---|---|---|
| NO. | Target | Sequence |
| 272 | IDO (SEQ ID | GACACCCUGCUGAAGGCCCUGCUGGAGAUCGCCAGCUGCCUGGAGAAGGCCCUGC |
| | NO: 2) | AGGUGUUC |
| 273 | PDL1 (SEQ ID | UUCAUGACCUACUGGCACCUGCUGAACGCCUUCACCGUGACCGUGCCCAAGGACC |
| | NO: 15) | UG |
| 274 | PDL2 (SEQ ID | AGCCUGGAGCUGCAGCUGCACCAGAUCGCCGCCCUGUUCACCGUGACCGUGCCCA |
| | NO: 102) | AGGAGCUG |
| 275 | PDL2 (SEQ ID | AUGAUCUUCCUGCUGCUGAUGCUGAGCCUGGAGCUGCAGCUGCACCAGAUCGCCG |
| | NO: 103) | CCCUGUUCACCGUGACCGUG |
| 276 | PDL2 (SEQ ID | CUGCUGCUGAUGCUGAGCCUGGAGCUG |
| | NO: 104) | |
| 277 | Arg1 (SEQ ID | AUCAGCGCCAAGGACAUCGUGUACAUCGGCCUGAGGGACGUGGACCCCGGCGAGC |
| | NO: 106) | ACUACAUCCUGAAGACCCUGGGCAUCAAGUACUUCAGCAUGACCGAGGUGGACAG |
| | | GCUG |
| 278 | Arg1 (SEQ ID | AUCAGCGCCAAGGACAUCGUGUACAUCGGCCUGAGGGACGUGGACCCCGGCGAGC |
| | NO: 107) | ACUACAUCCUGAAGACCCUGGGCAUCAAGUACUUCAGCAUG |
| 279 | Arg1 (SEQ ID | AUCAGCGCCAAGGACAUCGUGUACAUCGGCCUGAGGGACGUGGACCCCGGCGAGC |
| | NO: 108) | ACUACAUCCUGAAGACCCUGGGCAUCAAGUACUUCAGCAUGACCGAGGUGGACAG |
| | | GCUGGGCAUCGGCAAG |
| 280 | Arg2 (SEQ ID | AGCCUGAGGGGCAGCCUGAGCAGGCUGCUGCAGACCAGGGUGCACAGCAUCCUGA |
| | NO: 160) | AGAAGAGCGUGCACAGCGUGGCCGUGAUCGGCGCCCCCUUCAGC |
| 281 | TGFb-15 (SEQ | AGGGAGGCCGUGCCCGAGCCCGUGCUGCUGAGCAGGGCCGAGCUGAGGCUGCUGA |
| | ID NO: 243) | GGCUG |

TABLE 3
DNA sequence counterparts to the predicted mRNA ORF sequences
described in Table 2

| SEQ | | |
|---|---|---|
| NO. | Target | Sequence |
| 282 | IDO (SEQ ID | gacaccctgctgaaggccctgctggagatcgccagctgcctggagaaggccctgc |
| | NO: 2) | aggtgttc |
| 283 | PDL1 (SEQ ID | ttcatgacctactggcacctgctgaacgccttcaccgtgaccgtgcccaaggacc |
| | NO: 15) | tg |
| 284 | PDL2 (SEQ ID | AGCCTGGAGCTGCAGCTGCACCAGATCGCCGCCCTGTTCACCGTGACCGTGCCCA |
| | NO: 102) | AGGAGCTG |
| 285 | PDL2 (SEQ ID | ATGATCTTCCTGCTGCTGATGCTGAGCCTGGAGCTGCAGCTGCACCAGATCGCCG |
| | NO: 103) | CCCTGTTCACCGTGACCGTG |
| 286 | PDL2 (SEQ ID | CTGCTGCTGATGCTGAGCCTGGAGCTG |
| | NO: 104) | |
| 287 | Arg1 (SEQ ID | ATCAGCGCCAAGGACATCGTGTACATCGGCCTGAGGGACGTGGACCCCGGCGAGC |
| | NO: 106) | ACTACATCCTGAAGACCCTGGGCATCAAGTACTTCAGCATGACCGAGGTGGACAG |
| | | GCTG |
| 288 | Arg1 (SEQ ID | ATCAGCGCCAAGGACATCGTGTACATCGGCCTGAGGGACGTGGACCCCGGCGAGC |
| | NO: 107) | ACTACATCCTGAAGACCCTGGGCATCAAGTACTTCAGCATG |
| 289 | Arg1 (SEQ ID | ATCAGCGCCAAGGACATCGTGTACATCGGCCTGAGGGACGTGGACCCCGGCGAGC |
| | NO: 108) | ACTACATCCTGAAGACCCTGGGCATCAAGTACTTCAGCATGACCGAGGTGGACAG |
| | | GCTGGGCATCGGCAAG |
| 290 | Arg2 (SEQ ID | AGCCTGAGGGGCAGCCTGAGCAGGCTGCTGCAGACCAGGGTGCACAGCATCCTGA |
| | NO: 160) | AGAAGAGCGTGCACAGCGTGGCCGTGATCGGCGCCCCCTTCAGC |
| 291 | TGFb-15 (SEQ | AGGGAGGCCGTGCCCGAGCCCGTGCTGCTGAGCAGGGCCGAGCTGAGGCTGCTGA |
| | ID NO: 243) | GGCTG |

The invention is illustrated by the following Examples, which should not be construed as limiting.

### EXAMPLES

### Example 1. Manufacture of Polynucleotides

Polynucleotides may be manufactured by any suitable method, including for example those disclosed in WO2014152027 (which is herein incorporated by reference in its entirety, together with each of its priority applications).

Polynucleotides may be purified by any suitable method, including for example those disclosed in WO2014152031 (which is herein incorporated by reference in its entirety, together with each of its priority applications).

Polynucleotides may be detected and characterised by any suitable method, including for example those disclosed in WO2014144039 (which is herein incorporated by reference in its entirety, together with each of its priority applications).

Polynucleotides may be characterised using a procedure selected from the group consisting of polynucleotide mapping, reverse transcriptase sequencing, charge distribution analysis, and detection of RNA impurities, wherein characterizing comprises determining the RNA transcript sequence, determining the purity of the RNA transcript, or determining the charge heterogeneity of the RNA transcript. Such methods are taught in, for example, WO2014144711 (which is herein incorporated by reference in its entirety, together with each of its priority applications).

### Example 2. Chimeric Polynucleotide Synthesis

Two regions or parts of a chimeric polynucleotide may be joined or ligated using triphosphate chemistry as described in Example 2 of WO2017020026 (which is herein incorporated by reference in its entirety, together with each of its priority applications).

### Example 3: PCR for cDNA Production

PCR procedures for the preparation of cDNA may be performed using 2×KAPA HIFI™ HotStart ReadyMix by Kapa Biosystems (Woburn, MA). This system includes 2×KAPA ReadyMix12.5 μl; Forward Primer (10 μM) 0.75 μl; Reverse Primer (10 μM) 0.75 μl; Template cDNA-100 ng; and dH₂O diluted to 25.0 μl. The reaction conditions are at 95° C. for 5 min. and 25 cycles of 98° C. for 20 sec, then 58° C. for 15 sec, then 72° C. for 45 sec, then 72° C. for 5 min. then 4° C. to termination.

The reaction may be cleaned up using Invitrogen's PURELINK™ PCR Micro Kit (Carlsbad, CA) per manufacturer's instructions (up to 5 μg). Larger reactions will require a cleanup using a product with a larger capacity. Following the cleanup, the cDNA is quantified using the NANODROP™ and analyzed by agarose gel electrophoresis to confirm the cDNA is the expected size. The cDNA is then submitted for sequencing analysis before proceeding to the in vitro transcription reaction.

### Example 4. In Vitro Transcription (IVT)

The in vitro transcription reaction generates polynucleotides containing uniformly modified polynucleotides. Such uniformly modified polynucleotides may comprise a region or part of the polynucleotides of the invention. The input nucleotide triphosphate (NTP) mix is made in-house using natural and un-natural NTPs.

A typical in vitro transcription reaction is described in Example 4 of WO2017020026 (which is herein incorporated by reference in its entirety, together with each of its priority applications). IVT may be used to generate mRNA of the invention from a suitable cDNA template.

### Example 5. MRNA for Vaccine Composition

The methods of Examples 1 to 4 are used to assemble mRNA comprising an ORF encoding at least one immunogenic peptide fragment of a polypeptide component of an immune system checkpoint, wherein the polypeptide component of the immune system checkpoint is selected from any one or more of the following:
a. IDO; and preferably wherein the immunogenic peptide fragment thereof is up to 50 consecutive amino acids of IDO, optionally comprising or consisting of the sequence of any one of SEQ ID NOs: 2 to 13, preferably SEQ ID NO: 2;
b. PDL1 or PDL2, preferably PDL1; and preferably wherein the immunogenic peptide fragment thereof is up to 50 consecutive amino acids of PDL1, optionally comprising or consisting of the sequence of any one of SEQ ID NOs: 15 to 100, preferably SEQ ID NOs: 15 or 16; or alternatively PDL2, and preferably wherein the immunogenic peptide fragment thereof is up to 50 consecutive amino acids of PDL2, optionally comprising or consisting of the sequence of any one of SEQ ID NOs: 102 to 104;
c. Arginase1 or Arginase2, preferably Arginase1; and preferably wherein the immunogenic peptide fragment thereof is up to 50 consecutive amino acids of Arginase1, optionally comprising or consisting of the sequence of any one of SEQ ID NOs: 106 to 158, preferably SEQ ID NO: 106; or alternatively Arginase2, and preferably wherein the immunogenic peptide fragment thereof is up to 50 consecutive amino acids of Arginase2, comprising or consisting of the sequence of any one of SEQ ID NOs: 160 to 220; preferably SEQ ID NO: 160;
d. TDO; and preferably wherein the immunogenic peptide fragment thereof is and preferably wherein the immunogenic peptide fragment thereof is up to 50 consecutive amino acids of TDO, optionally comprising or consisting of the sequence of any one of SEQ ID NOs: 222 to 238; or
e. TGFb; and preferably wherein the immunogenic peptide fragment thereof is and preferably wherein the immunogenic peptide fragment thereof is up to 50 consecutive amino acids of TGFb, optionally comprising or consisting of the sequence of any one of SEQ ID NOs: 240 to 271.

The methods of Examples 1 to 4 are used to assemble mRNA comprising an ORF which comprises:
at least one copy of a nucleic acid encoding SEQ ID NO: 2 (IO102) and at least one copy of a nucleic acid encoding SEQ ID NO: 15 (IO103) and/or 16 (IO104.1);
at least one copy of a nucleic acid encoding SEQ ID NO: 2 (IO102) and at least one copy of a nucleic acid encoding SEQ ID NO: 106 (IO112);
at least one copy of a nucleic acid encoding SEQ ID NO: 15 (IO103) and/or 16 (IO104.1) and at least one copy of a nucleic acid encoding SEQ ID NO: 106 (IO112);
at least one copy of a nucleic acid encoding SEQ ID NO: 2 (IO102) and at least one copy of a nucleic acid encoding SEQ ID NO: 15 (IO103) and/or 16 (IO104.1) and at least one copy of a nucleic acid encoding SEQ ID NO: 106 (IO112).

The mRNA is formulated into a vaccine composition by any suitable method, including in particular formulation with a lipid nanoparticle carrier comprising a molar ratio of about 20-60% cationic lipid:5-25% non-cationic lipid:25-55% sterol; and 0.5-15% PEG-modified lipid.

### Example 6. Clinical Formulation of Lipoplexes

The following describes the clinical formulation of lipoplexes as described in Example 12 of U.S. Pat. No. 10,485,884 (which is herein incorporated by reference in its entirety). The formulation comprises two steps, namely the preformulation of a given RNA by using isotonic sodium chloride solution as diluent and the lipoplex formation by adding a defined amount of liposomes. For preformulation, first 4 ml sodium chloride (0.9% w/w in water) solution will be taken out of the NaCl vial by a syringe and added to the RNA. Then, 400 μL of liposomes (2.8 mg/mL total lipid in water) will be taken out of the liposome vial and injected using a cannula (inner diameter of 0.9 mm) into the solution of RNA and sodium chloride. The obtained RNA lipoplex formulation (5.5 ml) can be administered either, by direct parenteral injection of the desired dose as well as after preparation of an intravenous infusion. To this end, from the RNA lipoplex formulation, 5.0 mL will be taken and diluted to an infusion bag containing 50 ml of isotonic sodium chloride solution. By this protocol, lipoplex formulations with particle sizes of about 300 to 500 nm are obtained in a robust and reproducible manner.

Materials and components which may be used are as follows:

### Components:

RNA: 0.5 mg/ml in 10 mM HEPES and 0.1 mM EDTA
Diluent: 0.9% NaCl
Liposomes: 2.68 mM DOTMA, 1.34 mM DOPE, particle size (Zave) 300-500 nm Syringes:
5 mL syringes: (e.g. Omnifix, 5 mL, Luer Lock, B. Braun Melsungen AG (Melsungen, Germany)
1 mL syringe: Injekt-F Tuberculin, 1 mL, Luer Lock, B. Braun Melsungen AG (Melsungen, Germany)

### Needles:

0.9×44 mm, 20 G 1½″, BD Microlance 3, Becton Dickinson S.A. (Fraga, Spain)

The sizes of the RNA lipoplex particles produced according to the above procedure range from 300 nm to 500 nm.

mRNA sequences described herein can be formulated in lipoplexes as described above. For example, any one of SEQ ID NOs: 272, 273, 274, 275, 276, 277, 278, 279, 280 and 281 can be formulated in the described lipoplex. Further, two or more different mRNA sequences can be formulated in the same lipoplex, e.g. SEQ ID NO: 272 and SEQ ID NO: 273.

### Example 7. Formulation of Modified mRNA Using Lipidoids

The following describes the formulation of modified mRNA using lipidoids as described in Example 8 of U.S. Pat. No. 10,898,571 (which is herein incorporated by reference in its entirety). Modified mRNAs (mmRNA) are formulated for in vitro experiments by mixing the mmRNA with the lipidoid at a set ratio prior to addition to cells. In vivo formulation may require the addition of extra ingredients to facilitate circulation throughout the body. To test the ability of these lipidoids to form particles suitable for in vivo work, a standard formulation process used for siRNA-lipidoid formulations is used as a starting point. Initial mmRNA-lipidoid formulations may consist of particles composed of 42% lipidoid, 48% cholesterol and 10% PEG, with further optimization of ratios possible. After formation of the particle, mmRNA is added and allowed to integrate with the complex. The encapsulation efficiency is determined using a standard dye exclusion assays.

### A. Lipid Synthesis

Six lipids, DLin-DMA, DLin-K-DMA, DLin-KC2-DMA, 98N12-5, C12-200 and DLin-MC3-DMA, are synthesized by methods outlined in the art in order to be formulated with modified RNA. DLin-DMA and precursors are synthesized as described in Heyes et. al, J. Control Release, 2005, 107, 276-287. DLin-K-DMA and DLin-KC2-DMA and precursors are synthesized as described in Semple et. al, Nature Biotechnology, 2010, 28, 172-176. 98N12-5 and precursor are synthesized as described in Akinc et. al, Nature Biotechnology, 2008, 26, 561-569.

C12-200 and precursors are synthesized according to the method outlined in Love et. al, PNAS, 2010, 107, 1864-1869. 2-epoxydodecane (5.10 g, 27.7 mmol, 8.2 eq) is added to a vial containing Amine 200 (0.723 g, 3.36 mmol, 1 eq) and a stirring bar. The vial is sealed and warmed to 80° C. The reaction is stirred for 4 days at 80° C. Then the mixture is purified by silica gel chromatography using a gradient from pure dichloromethane (DCM) to DCM:MeOH 98:2. The target compound is further purified by RP-HPLC to afford the desired compound.

DLin-MC3-DMA and precursors are synthesized according to procedures described in WO 2010054401 herein incorporated by reference in its entirety. A mixture of dilinoleyl methanol (1.5 g, 2.8 mmol, 1 eq), N,N-dimethylaminobutyric acid (1.5 g, 2.8 mmol, 1 eq), DIPEA (0.73 mL, 4.2 mmol, 1.5 eq) and TBTU (1.35 g, 4.2 mmol, 1.5 eq) in 10 mL of DMF is stirred for 10 h at room temperature. Then the reaction mixture is diluted in ether and washed with water. The organic layer is dried over anhydrous sodium sulfate, filtrated and concentrated under reduced pressure. The crude product is purified by silica gel chromatography using a gradient DCM to DCM:MeOH 98:2. Subsequently the target compound is subjected to an additional RP-HPLC purification which is done using a YMC-Pack C4 column to afford the target compound.

### B. Formulation of Modified RNA Nanoparticles

Solutions of synthesized lipid, 1,2-distearoyl-3-phosphatidylcholine (DSPC) (Avanti Polar Lipids, Alabaster, Ala.), cholesterol (Sigma-Aldrich, Taufkirchen, Germany), and α-[3′-(1,2-dimyristoyl-3-propanoxy)-carboxamide-propyl]-ω-methoxy-polyoxyethylene (PEG-c-DOMG) (NOF, Bouwelven, Belgium) are prepared at concentrations of 50 mM in ethanol and stored at −20° C. The lipids are combined to yield molar ratio of 50:10:38.5:1.5 (Lipid:DSPC:Cholesterol:PEG-c-DOMG) and diluted with ethanol to a final lipid concentration of 25 mM. Solutions of modified mRNA at a concentration of 1-2 mg/mL in water are diluted in 50 mM sodium citrate buffer at a pH of 3 to form a stock modified mRNA solution. Formulations of the lipid and modified mRNA are prepared by combining the synthesized lipid solution with the modified mRNA solution at total lipid to modified mRNA weight ratio of 10:1, 15:1, 20:1 and 30:1. The lipid ethanolic solution is rapidly injected into aqueous modified mRNA solution to afford a suspension containing 33% ethanol. The solutions are injected either manually (MI) or by the aid of a syringe pump (SP) (Harvard Pump 33 Dual Syringe Pump Harvard Apparatus Holliston, Mass.). To remove the ethanol and to achieve the buffer exchange, the formulations are dialyzed twice against phosphate buffered saline (PBS), pH 7.4 at volumes 200-times of the primary product using a Slide-A-Lyzer cassettes (Thermo Fisher Scientific Inc. Rockford, Ill.) with a molecular weight cutoff (MWCO) of 10 kD. The first dialysis is carried at room temperature for 3 hours and then the formulations were dialyzed overnight at 4° C. The resulting nanoparticle suspension was filtered through 0.2 μm sterile filter (Sarstedt, Niimbrecht, Germany) into glass vials and sealed with a crimp closure.

### C. Characterization of Formulations

A Zetasizer Nano ZS (Malvern Instruments Ltd, Malvern, Worcestershire, UK) is used to determine the particle size, the polydispersity index (PDI) and the zeta potential of the modified mRNA nanoparticles in 1×PBS in determining particle size and 15 mM PBS in determining zeta potential.

Ultraviolet-visible spectroscopy is used to determine the concentration of modified mRNA nanoparticle formulation. 100 μL of the diluted formulation in 1×PBS is added to 900 μL of a 4:1 (v/v) mixture of methanol and chloroform. After mixing, the absorbance spectrum of the solution is recorded between 230 nm and 330 nm on a DU 800 spectrophotometer (Beckman Coulter, Beckman Coulter, Inc., Brea, Calif.). The modified RNA concentration in the nanoparticle formulation is calculated based on the extinction coefficient of the modified RNA used in the formulation and on the difference between the absorbance at a wavelength of 260 nm and the baseline value at a wavelength of 330 nm.

QUANT-IT™ RIBOGREEN® RNA assay (Invitrogen Corporation Carlsbad, Calif.) is used to evaluate the encapsulation of modified RNA by the nanoparticle. The samples are diluted to a concentration of approximately 5 μg/mL in TE buffer (10 mM Tris-HCl, 1 mM EDTA, pH 7.5). 50 μL of the diluted samples are transferred to a polystyrene 96 well plate, then either 50 μL of TE buffer or 50 μL of a 2% Triton X-100 solution is added. The plate is incubated at a temperature of 37° C. for 15 minutes. The RIBOGREEN® reagent is diluted 1:100 in TE buffer, 100 μL of this solution is added to each well. The fluorescence intensity is measured using a fluorescence plate reader (Wallac Victor 1420 Multilablel Counter; Perkin Elmer, Waltham, Mass.) at an excitation wavelength of ˜480 nm and an emission wavelength of ˜520 nm. The fluorescence values of the reagent blank are subtracted from that of each of the samples and the percentage of free modified RNA is determined by dividing the fluorescence intensity of the intact sample (without addition of Triton X-100) by the fluorescence value of the disrupted sample (caused by the addition of Triton X-100).

### D. In Vitro Incubation

Human embryonic kidney epithelial (HEK293) and hepatocellular carcinoma epithelial (HepG2) cells (LGC standards GmbH, Wesel, Germany) are seeded on 96-well plates (Greiner Bio-one GmbH, Frickenhausen, Germany) and plates for HEK293 cells are precoated with collagen type1. HEK293 are seeded at a density of 30,000 and HepG2 are seeded at a density of 35,000 cells per well in 100 μl cell culture medium. For HEK293 the cell culture medium is DMEM, 10% FCS, adding 2 mM L-Glutamine, 1 mM Sodiumpyruvate and 1× non-essential amino acids (Biochrom AG, Berlin, Germany) and 1.2 mg/ml Sodiumbicarbonate (Sigma-Aldrich, Munich, Germany) and for HepG2 the culture medium is MEM (Gibco Life Technologies, Darmstadt, Germany), 10% FCS adding 2 mM L-Glutamine, 1 mM Sodiumpyruvate and 1× non-essential amino acids (Biochrom AG, Berlin, Germany. Formulations containing mRNA (mRNA sequence selected from SEQ ID NO: 272-281; poly-A tail of approximately 160 nucleotides not shown in sequence; 5′ cap, Cap1) are added in quadruplicates directly after seeding the cells and incubated.

1. An mRNA comprising:
An open reading frame (ORF) encoding at least one immunogenic peptide fragment of a polypeptide component of an immune system checkpoint;
A 5′ terminal cap at the 5′ end;
A 5′ untranslated region (UTR) which is included 5′ of the ORF;
A 3′ UTR which is included 3′ of the ORF; and
A 3′ tailing sequence at the 3′ end.

2. The mRNA of claim 1, wherein the ORF encodes at least 2, 3, 4, 5, 10 or more immunogenic peptide fragments which are not identical to each other and wherein each may be:
(i) a different part of the same immune system checkpoint component polypeptide; or
(ii) a fragment of a different immune system checkpoint component polypeptide, which is optionally a component of the same or of a different immune system checkpoint.

3. The mRNA of any one of the preceding claims, wherein the ORF includes multiple copies of each sequence encoding an immunogenic peptide fragment, optionally at least 2, 3, 4, 5, 10, 20, 30, 40, 50 or more copies of each said sequence.

4. The mRNA of any one of the preceding claims, wherein the sequences encoding an immunogenic peptide fragment are each interspersed by a sequence encoding a cleavage sensitive site, preferably a cathepsin B cleavage site.

5. The mRNA of any one of the preceding claims wherein said ORF is codon optimised for human expression and/or to reduce immune recognition.

6. The mRNA of any one of the preceding claims comprising at least one chemical modification, optionally selected from pseudouridine, NI-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and 2′-O-methyl uridine.

7. The mRNA of any one of the preceding claims wherein:
a. Said 5′ cap is a 7-methylguanylate cap, preferably m7G(5′)ppp(5′)NlmpNp
b. Said tailing sequence is a polyA tail, a polyA-G quartet and/or a stem loop sequence, preferably a polyA tail, typically of between 40 and 200 nucleotides in length.

8. The mRNA of any one of the preceding claims, wherein the immune system checkpoint is selected from any one or more of the following:
a. The interaction between IDO1 and its substrate;
b. The interaction between PD1 and PDL1 and/or PD1 and PDL2;
c. The interaction between Arginase1 or Arginase 2 and its substrate;
d. The interaction between TDO and its substrate;
e. The interaction between TGFb1 and its receptors;
f. The interaction between CTLA4 and CD86 and/or CTLA4 and CD80;
g. The interaction between B7-H3 and/or B7-H4 and their respective ligands;
h. The interaction between HVEM and BTLA;
i. The interaction between GAL9 and TIM3;
j. The interaction between MHC class I or II and LAG3; and
k. The interaction between MHC class I or II and KIR.

9. The mRNA of any one of the preceding claims, wherein the polypeptide component of the immune system checkpoint is selected from any one or more of the following:
a. IDO; and preferably wherein the immunogenic peptide fragment thereof is up to 50 consecutive amino acids of IDO, optionally comprising or consisting of the sequence of any one of SEQ ID NOs: 2 to 13, preferably SEQ ID NO: 2;
b. PDL1 or PDL2, preferably PDL1; and preferably wherein the immunogenic peptide fragment thereof is up to 50 consecutive amino acids of PDL1, optionally comprising or consisting of the sequence of any one of SEQ ID NOs: 15 to 100, preferably SEQ ID NOs: 15 or 16; or alternatively PDL2, and preferably wherein the immunogenic peptide fragment thereof is up to 50 consecutive amino acids of PDL2, optionally comprising or consisting of the sequence of any one of SEQ ID NOs: 102 to 104;
c. Arginase1 or Arginase2, preferably Arginase1; and preferably wherein the immunogenic peptide fragment thereof is up to 50 consecutive amino acids of Arginase1, optionally comprising or consisting of the sequence of any one of SEQ ID NOs: 106 to 158, preferably SEQ ID NO: 106; or alternatively Arginase2, and preferably wherein the immunogenic peptide fragment thereof is up to 50 consecutive amino acids of Arginase2, comprising or consisting of the sequence of any one of SEQ ID NOs: 160 to 220; preferably SEQ ID NO: 160;
d. TDO; and preferably wherein the immunogenic peptide fragment thereof is and preferably wherein the immunogenic peptide fragment thereof is up to 50 consecutive amino acids of TDO, optionally comprising or consisting of the sequence of any one of SEQ ID NOs: 222 to 238; or
e. TGFb; and preferably wherein the immunogenic peptide fragment thereof is and preferably wherein the immunogenic peptide fragment thereof is up to 50 consecutive amino acids of TGFb, optionally comprising or consisting of the sequence of any one of SEQ ID NOs: 240 to 271.

10. The mRNA of claim 9, wherein the ORF encodes:
At least one immunogenic polypeptide fragment of (a) and (b);
At least one immunogenic polypeptide fragment of (a) and (c);
At least one immunogenic polypeptide fragment of (a) and (d);
At least one immunogenic polypeptide fragment of (a) and (e);
At least one immunogenic polypeptide fragment of (b) and (c);
At least one immunogenic polypeptide fragment of (b) and (d);
At least one immunogenic polypeptide fragment of (b) and (e);
At least one immunogenic polypeptide fragment of (c) and (d);
At least one immunogenic polypeptide fragment of (c) and (e);
At least one immunogenic polypeptide fragment of (d) and (e); or
At least one immunogenic polypeptide fragment of (a), (b) and (c).

11. The mRNA of any one of claims 1-10, wherein the ORF comprises:
at least one copy of a nucleic acid encoding SEQ ID NO: 2 (IO102) and at least one copy of a nucleic acid encoding SEQ ID NO: 15 (IO103) and/or 16 (IO104.1);
at least one copy of a nucleic acid encoding SEQ ID NO: 2 (IO102) and at least one copy of a nucleic acid encoding SEQ ID NO: 106 (IO112);
at least one copy of a nucleic acid encoding SEQ ID NO: 15 (IO103) and/or 16 (IO104.1) and at least one copy of a nucleic acid encoding SEQ ID NO: 106 (IO112); or
at least one copy of a nucleic acid encoding SEQ ID NO: 2 (IO102) and at least one copy of a nucleic acid encoding SEQ ID NO: 15 (IO103) and/or 16 (IO104.1) and at least one copy of a nucleic acid encoding SEQ ID NO: 106 (IO112).

12. The mRNA of any one of claims 1-11, wherein the ORF comprises one or more of SEQ ID NO: 272, 273, 274, 275, 276, 277, 278, 279, 280 and 281.

13. The mRNA of any one of claim 1-12, wherein the ORF comprises:
at least one copy of SEQ ID NO: 272 (IO102) and at least one copy of SEQ ID NO: 273 (IO103);
at least one copy of SEQ ID NO: 272 (IO102) and at least one copy of SEQ ID NO: 277 (IO112);
one copy of SEQ ID NO: 273 (IO103) and at least one copy of SEQ ID NO: 277 (IO112); or
one copy of SEQ ID NO: 272 (IO102) and at least one copy of SEQ ID NO: 273 (IO103) and at least one copy of SEQ ID NO: 277 (IO112).

14. A vaccine composition comprising the mRNA of any one of claims 1-13, formulated in a lipid nanoparticle composition, optionally wherein the lipid nanoparticle has a mean diameter of 50-200 nm.

15. The vaccine composition according to claim 14, wherein the lipid nanoparticle composition comprises a cationic lipid, a PEG-modified lipid, a sterol, and a non-cationic lipid, optionally wherein the lipid nanoparticle carrier comprises a molar ratio of about 20-60% cationic lipid:5-25% non-cationic lipid:25-55% sterol; and 0.5-15% PEG-modified lipid.

16. The vaccine composition of claim 14 or 15, further comprising an adjuvant.

17. A vaccine composition comprising a first and a second mRNA,
wherein the first mRNA comprises a first open reading frame (ORF) encoding a first immunogenic peptide fragment of a polypeptide component of an immune system checkpoint and wherein the second mRNA comprises a second ORF encoding a second immunogenic peptide fragment of a polypeptide component of an immune system checkpoint,
wherein the first and second ORF each comprise
A 5′ terminal cap at the 5′ end;
A 5′ untranslated region (UTR) which is included 5′ of the ORF;
A 3′ UTR which is included 3′ of the ORF; and
A 3′ tailing sequence at the 3′ end.

18. The vaccine composition of claim 17, wherein:
the first immunogenic polypeptide fragment is IDO; and preferably wherein the immunogenic peptide fragment thereof is up to 50 consecutive amino acids of IDO, optionally comprising or consisting of the sequence of any one of SEQ ID NOs: 2 to 13, preferably SEQ ID NO: 2; and the second immunogenic polypeptide fragment is PDL1 or PDL2, preferably PDL1; and preferably wherein the immunogenic peptide fragment thereof is up to 50 consecutive amino acids of PDL1, optionally comprising or consisting of the sequence of any one of SEQ ID NOs: 15 to 100, preferably SEQ ID NOs: 15 or 16; or alternatively PDL2, and preferably wherein the immunogenic peptide fragment thereof is up to 50 consecutive amino acids of PDL2, optionally comprising or consisting of the sequence of any one of SEQ ID NOs: 102 to 104;
the first immunogenic polypeptide fragment is IDO; and preferably wherein the immunogenic peptide fragment thereof is up to 50 consecutive amino acids of IDO, optionally comprising or consisting of the sequence of any one of SEQ ID NOs: 2 to 13, preferably SEQ ID NO: 2; and the second immunogenic polypeptide fragment is Arginase1 or Arginase2, preferably Arginase1; and preferably wherein the immunogenic peptide fragment thereof is up to 50 consecutive amino acids of Arginase1, optionally comprising or consisting of the sequence of any one of SEQ ID NOs: 106 to 158, preferably SEQ ID NO: 106; or alternatively Arginase2, and preferably wherein the immunogenic peptide fragment thereof is up to 50 consecutive amino acids of Arginase2, comprising or consisting of the sequence of any one of SEQ ID NOs: 160 to 220; preferably SEQ ID NO: 160;
the first immunogenic polypeptide fragment is IDO; and preferably wherein the immunogenic peptide fragment thereof is up to 50 consecutive amino acids of IDO, optionally comprising or consisting of the sequence of any one of SEQ ID NOs: 2 to 13, preferably SEQ ID NO: 2; and the second immunogenic polypeptide fragment is TDO; and preferably wherein the immunogenic peptide fragment thereof is and preferably wherein the immunogenic peptide fragment thereof is up to 50 consecutive amino acids of TDO, optionally comprising or consisting of the sequence of any one of SEQ ID NOs: 222 to 238;
the first immunogenic polypeptide fragment is IDO; and preferably wherein the immunogenic peptide fragment thereof is up to 50 consecutive amino acids of IDO, optionally comprising or consisting of the sequence of any one of SEQ ID NOs: 2 to 13, preferably SEQ ID NO: 2; and the second immunogenic polypeptide fragment is TGFb; and preferably wherein the immunogenic peptide fragment thereof is and preferably wherein the immunogenic peptide fragment thereof is up to 50 consecutive amino acids of TGFb, optionally comprising or consisting of the sequence of any one of SEQ ID NOs: 240 to 271;
the first immunogenic polypeptide fragment is PDL1 or PDL2, preferably PDL1; and preferably wherein the immunogenic peptide fragment thereof is up to 50 consecutive amino acids of PDL1, optionally comprising or consisting of the sequence of any one of SEQ ID NOs: 15 to 100, preferably SEQ ID NOs: 15 or 16; or alternatively PDL2, and preferably wherein the immunogenic peptide fragment thereof is up to 50 consecutive amino acids of PDL2, optionally comprising or consisting of the sequence of any one of SEQ ID NOs: 102 to 104; and the second immunogenic polypeptide fragment is Arginase1 or Arginase2, preferably Arginase1; and preferably wherein the immunogenic peptide fragment thereof is up to 50 consecutive amino acids of Arginase1, optionally comprising or consisting of the sequence of any one of SEQ ID NOs: 106 to 158, preferably SEQ ID NO: 106; or alternatively Arginase2, and preferably wherein the immunogenic peptide fragment thereof is up to 50 consecutive amino acids of Arginase2, comprising or consisting of the sequence of any one of SEQ ID NOs: 160 to 220; preferably SEQ ID NO: 160;
the first immunogenic polypeptide fragment is PDL1 or PDL2, preferably PDL1; and preferably wherein the immunogenic peptide fragment thereof is up to 50 consecutive amino acids of PDL1, optionally comprising or consisting of the sequence of any one of SEQ ID NOs: 15 to 100, preferably SEQ ID NOs: 15 or 16; or alternatively PDL2, and preferably wherein the immunogenic peptide fragment thereof is up to 50 consecutive amino acids of PDL2, optionally comprising or consisting of the sequence of any one of SEQ ID NOs: 102 to 104; and the second immunogenic polypeptide fragment is TDO; and preferably wherein the immunogenic peptide fragment thereof is and preferably wherein the immunogenic peptide fragment thereof is up to 50 consecutive amino acids of TDO, optionally comprising or consisting of the sequence of any one of SEQ ID NOs: 222 to 238;
the first immunogenic polypeptide fragment is PDL1 or PDL2, preferably PDL1; and preferably wherein the immunogenic peptide fragment thereof is up to 50 consecutive amino acids of PDL1, optionally comprising or consisting of the sequence of any one of SEQ ID NOs: 15 to 100, preferably SEQ ID NOs: 15 or 16; or alternatively PDL2, and preferably wherein the immunogenic peptide fragment thereof is up to 50 consecutive amino acids of PDL2, optionally comprising or consisting of the sequence of any one of SEQ ID NOs: 102 to 104; and the second immunogenic polypeptide fragment is TGFb; and preferably wherein the immunogenic peptide fragment thereof is and preferably wherein the immunogenic peptide fragment thereof is up to 50 consecutive amino acids of TGFb, optionally comprising or consisting of the sequence of any one of SEQ ID NOs: 240 to 271;
the first immunogenic polypeptide fragment is Arginase1 or Arginase2, preferably Arginase1; and preferably wherein the immunogenic peptide fragment thereof is up to 50 consecutive amino acids of Arginase1, optionally comprising or consisting of the sequence of any one of SEQ ID NOs: 106 to 158, preferably SEQ ID NO: 106; or alternatively Arginase2, and preferably wherein the immunogenic peptide fragment thereof is up to 50 consecutive amino acids of Arginase2, comprising or consisting of the sequence of any one of SEQ ID NOs: 160 to 220; preferably SEQ ID NO: 160; and the second immunogenic polypeptide fragment is TDO; and preferably wherein the immunogenic peptide fragment thereof is and preferably wherein the immunogenic peptide fragment thereof is up to 50 consecutive amino acids of TDO, optionally comprising or consisting of the sequence of any one of SEQ ID NOs: 222 to 238;
the first immunogenic polypeptide fragment is Arginase1 or Arginase2, preferably Arginase1; and preferably wherein the immunogenic peptide fragment thereof is up to 50 consecutive amino acids of Arginase1, optionally comprising or consisting of the sequence of any one of SEQ ID NOs: 106 to 158, preferably SEQ ID NO: 106; or alternatively Arginase2, and preferably wherein the immunogenic peptide fragment thereof is up to 50 consecutive amino acids of Arginase2, comprising or consisting of the sequence of any one of SEQ ID NOs: 160 to 220; preferably SEQ ID NO: 160; and the second immunogenic polypeptide fragment is TGFb; and preferably wherein the immunogenic peptide fragment thereof is and preferably wherein the immunogenic peptide fragment thereof is up to 50 consecutive amino acids of TGFb, optionally comprising or consisting of the sequence of any one of SEQ ID NOs: 240 to 271; or
the first immunogenic polypeptide fragment is TDO; and preferably wherein the immunogenic peptide fragment thereof is and preferably wherein the immunogenic peptide fragment thereof is up to 50 consecutive amino acids of TDO, optionally comprising or consisting of the sequence of any one of SEQ ID NOs: 222 to 238; and the second immunogenic polypeptide fragment is TGFb; and preferably wherein the immunogenic peptide fragment thereof is and preferably wherein the immunogenic peptide fragment thereof is up to 50 consecutive amino acids of TGFb, optionally comprising or consisting of the sequence of any one of SEQ ID NOs: 240 to 271.

19. The vaccine composition of claim 18, further comprising a third mRNA comprising a third open reading frame (ORF) encoding a third immunogenic peptide fragment of a polypeptide component of an immune system checkpoint.

20. The vaccine composition of claim 19, wherein:
the first immunogenic polypeptide fragment is IDO; and preferably wherein the immunogenic peptide fragment thereof is up to 50 consecutive amino acids of IDO, optionally comprising or consisting of the sequence of any one of SEQ ID NOs: 2 to 13, preferably SEQ ID NO: 2;
the second immunogenic polypeptide fragment is PDL1 or PDL2, preferably PDL1; and preferably wherein the immunogenic peptide fragment thereof is up to 50 consecutive amino acids of PDL1, optionally comprising or consisting of the sequence of any one of SEQ ID NOs: 15 to 100, preferably SEQ ID NOs: 15 or 16; or alternatively PDL2, and preferably wherein the immunogenic peptide fragment thereof is up to 50 consecutive amino acids of PDL2, optionally comprising or consisting of the sequence of any one of SEQ ID NOs: 102 to 104; and
the third immunogenic polypeptide fragment is Arginase1 or Arginase2, preferably Arginase1; and preferably wherein the immunogenic peptide fragment thereof is up to 50 consecutive amino acids of Arginase1, optionally comprising or consisting of the sequence of any one of SEQ ID NOs: 106 to 158, preferably SEQ ID NO: 106; or alternatively Arginase2, and preferably wherein the immunogenic peptide fragment thereof is up to 50 consecutive amino acids of Arginase2, comprising or consisting of the sequence of any one of SEQ ID NOs: 160 to 220; preferably SEQ ID NO: 150.

21. The vaccine composition of claim 18 or 19, wherein:
the first ORF comprises at least one copy of a nucleic acid encoding SEQ ID NO: 2 (IO102) and the second ORF comprises at least one copy of a nucleic acid encoding SEQ ID NO: 15 (IO103) and/or 16 (IO104.1);
the first ORF comprises at least one copy of a nucleic acid encoding SEQ ID NO: 2 (IO102) and the second ORF comprises at least one copy of a nucleic acid encoding SEQ ID NO: 106 (IO112);
the first ORF comprises at least one copy of a nucleic acid encoding SEQ ID NO: 15 (IO103) and/or 16 (IO104.1) and the second ORF comprises at least one copy of a nucleic acid encoding SEQ ID NO: 106 (IO112); or
the first ORF comprises at least one copy of a nucleic acid encoding SEQ ID NO: 2 (IO102) and the second ORF comprises at least one copy of a nucleic acid encoding SEQ ID NO: 15 (IO103) and/or 16 (IO104.1) and the third ORF comprises at least one copy of a nucleic acid encoding SEQ ID NO: 106 (IO112).

22. The vaccine composition of any one of claims 17-21, wherein the first, second, and/or third ORF each comprise one or more of SEQ ID NOs: 272, 273, 274, 275, 276, 277, 278, 279, 280 and 281.

23. The vaccine composition of any one of claims 17-21, wherein:
the first ORF comprises at least one copy of SEQ ID NO: 272 (IO102) and the second ORF comprises at least one copy of SEQ ID NO: 273 (IO103);
the first ORF comprises at least one copy of SEQ ID NO: 272 (IO102) and the second ORF comprises at least one copy of SEQ ID NO: 277 (IO112);
the first ORF comprises one copy of SEQ ID NO: 273 (IO103) and the second ORF comprises at least one copy of SEQ ID NO: 277 (IO112); or
the first ORF comprises one copy of SEQ ID NO: 272 (IO102) and the second ORF comprises at least one copy of SEQ ID NO: 273 (IO103), and the third ORF comprises at least one copy of SEQ ID NO: 277 (IO112).

24. A method of treating or preventing a disease comprising administering to a patient in need thereof a therapeutically or prophylactically effective amount of an mRNA according to any one of claims 1 to 13, or a vaccine composition according to any one of claims 14 to 23.

25. The method of claim 24 wherein the disease is cancer.


SEQUENCE LISTING (291 sequences)

SEQ ID NO: 1 - protein, 403 aa, synthetic construct [sequence omitted]

SEQ ID NO: 2 - protein, 21 aa, synthetic construct
DTLLKALLEIASCLEKALQVF

SEQ ID NO: 3 - protein, 9 aa, synthetic construct
ALLEIASCL

SEQ ID NO: 4 - protein, 9 aa, synthetic construct
QLRERVEKL

SEQ ID NO: 5 - protein, 9 aa, synthetic construct
FLVSLLVEI

SEQ ID NO: 6 - protein, 9 aa, synthetic construct
TLLKALLEI

SEQ ID NO: 7 - protein, 9 aa, synthetic construct
FIAKHLPDL

SEQ ID NO: 8 - protein, 9 aa, synthetic construct
VLSKGDAGL

SEQ ID NO: 9 - protein, 9 aa, synthetic construct
DLMNFLKTV

SEQ ID NO: 10 - protein, 9 aa, synthetic construct
VLLGIQQTA

SEQ ID NO: 11 - protein, 9 aa, synthetic construct
KVLPRNIAV

SEQ ID NO: 12 - protein, 10 aa, synthetic construct
KLNMLSIDHL

SEQ ID NO: 13 - protein, 10 aa, synthetic construct
SLRSYHLQIV

SEQ ID NO: 14 - protein, 290 aa, synthetic construct
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 15 - protein, 19 aa, synthetic construct
FMTYWHLLNAFTVTVPKDL

SEQ ID NO: 16 - protein, 28 aa, synthetic construct
RTHLVILGAILLCLGVALTFIFRLRKGR

SEQ ID NO: 17 - protein, 23 aa, synthetic construct
VILGAILLCLGVALTFIFRLRKG

SEQ ID NO: 18 - protein, 9 aa, synthetic construct
LLNAFTVTV

SEQ ID NO: 19 - protein, 10 aa, synthetic construct
FIFMTYWHLL

SEQ ID NO: 20 - protein, 9 aa, synthetic construct
LLNAFTVTV

SEQ ID NO: 21 - protein, 9 aa, synthetic construct
ILLCLGVAL

SEQ ID NO: 22 - protein, 9 aa, synthetic construct
ILGAILLCL

SEQ ID NO: 23 - protein, 9 aa, synthetic construct
HLVILGAIL

SEQ ID NO: 24 - protein, 9 aa, synthetic construct
CLGVALTFI

SEQ ID NO: 25 - protein, 10 aa, synthetic construct
VILGAILLCL

SEQ ID NO: 26 - protein, 17 aa, synthetic construct
VILGAILLCLGVALTFI

SEQ ID NO: 27 - protein, 18 aa, synthetic construct
VILGAILLCLGVALTFIF

SEQ ID NO: 28 - protein, 19 aa, synthetic construct
VILGAILLCLGVALTFIFR

SEQ ID NO: 29 - protein, 20 aa, synthetic construct
VILGAILLCLGVALTFIFRL

SEQ ID NO: 30 - protein, 21 aa, synthetic construct
VILGAILLCLGVALTFIFRLR

SEQ ID NO: 31 - protein, 22 aa, synthetic construct
VILGAILLCLGVALTFIFRLRK

SEQ ID NO: 32 - protein, 24 aa, synthetic construct
VILGAILLCLGVALTFIFRLRKGR

SEQ ID NO: 33 - protein, 25 aa, synthetic construct
VILGAILLCLGVALTFIFRLRKGRM

SEQ ID NO: 34 - protein, 26 aa, synthetic construct
VILGAILLCLGVALTFIFRLRKGRMM

SEQ ID NO: 35 - protein, 27 aa, synthetic construct
VILGAILLCLGVALTFIFRLRKGRMMD

SEQ ID NO: 36 - protein, 18 aa, synthetic construct
LVILGAILLCLGVALTFI

SEQ ID NO: 37 - protein, 19 aa, synthetic construct
LVILGAILLCLGVALTFIF

SEQ ID NO: 38 - protein, 20 aa, synthetic construct
LVILGAILLCLGVALTFIFR

SEQ ID NO: 39 - protein, 21 aa, synthetic construct
LVILGAILLCLGVALTFIFRL

SEQ ID NO: 40 - protein, 22 aa, synthetic construct
LVILGAILLCLGVALTFIFRLR

SEQ ID NO: 41 - protein, 23 aa, synthetic construct
LVILGAILLCLGVALTFIFRLRK

SEQ ID NO: 42 - protein, 24 aa, synthetic construct
LVILGAILLCLGVALTFIFRLRKG

SEQ ID NO: 43 - protein, 25 aa, synthetic construct
LVILGAILLCLGVALTFIFRLRKGR

SEQ ID NO: 44 - protein, 26 aa, synthetic construct
LVILGAILLCLGVALTFIFRLRKGRM

SEQ ID NO: 45 - protein, 27 aa, synthetic construct
LVILGAILLCLGVALTFIFRLRKGRMM

SEQ ID NO: 46 - protein, 28 aa, synthetic construct
LVILGAILLCLGVALTFIFRLRKGRMMD

SEQ ID NO: 47 - protein, 19 aa, synthetic construct
HLVILGAILLCLGVALTFI

SEQ ID NO: 48 - protein, 20 aa, synthetic construct
HLVILGAILLCLGVALTFIF

SEQ ID NO: 49 - protein, 21 aa, synthetic construct
HLVILGAILLCLGVALTFIFR

SEQ ID NO: 50 - protein, 22 aa, synthetic construct
HLVILGAILLCLGVALTFIFRL

SEQ ID NO: 51 - protein, 23 aa, synthetic construct
HLVILGAILLCLGVALTFIFRLR

SEQ ID NO: 52 - protein, 24 aa, synthetic construct
HLVILGAILLCLGVALTFIFRLRK

SEQ ID NO: 53 - protein, 25 aa, synthetic construct
HLVILGAILLCLGVALTFIFRLRKG

SEQ ID NO: 54 - protein, 26 aa, synthetic construct
HLVILGAILLCLGVALTFIFRLRKGR

SEQ ID NO: 55 - protein, 27 aa, synthetic construct
HLVILGAILLCLGVALTFIFRLRKGRM

SEQ ID NO: 56 - protein, 28 aa, synthetic construct
HLVILGAILLCLGVALTFIFRLRKGRMM

SEQ ID NO: 57 - protein, 29 aa, synthetic construct
HLVILGAILLCLGVALTFIFRLRKGRMMD

SEQ ID NO: 58 - protein, 20 aa, synthetic construct
THLVILGAILLCLGVALTFI

SEQ ID NO: 59 - protein, 21 aa, synthetic construct
THLVILGAILLCLGVALTFIF

SEQ ID NO: 60 - protein, 22 aa, synthetic construct
THLVILGAILLCLGVALTFIFR

SEQ ID NO: 61 - protein, 23 aa, synthetic construct
THLVILGAILLCLGVALTFIFRL

SEQ ID NO: 62 - protein, 24 aa, synthetic construct
THLVILGAILLCLGVALTFIFRLR

SEQ ID NO: 63 - protein, 25 aa, synthetic construct
THLVILGAILLCLGVALTFIFRLRK

SEQ ID NO: 64 - protein, 26 aa, synthetic construct
THLVILGAILLCLGVALTFIFRLRKG

SEQ ID NO: 65 - protein, 27 aa, synthetic construct
THLVILGAILLCLGVALTFIFRLRKGR

SEQ ID NO: 66 - protein, 28 aa, synthetic construct
THLVILGAILLCLGVALTFIFRLRKGRM

SEQ ID NO: 67 - protein, 29 aa, synthetic construct
THLVILGAILLCLGVALTFIFRLRKGRMM

SEQ ID NO: 68 - protein, 30 aa, synthetic construct
THLVILGAILLCLGVALTFIFRLRKGRMMD

SEQ ID NO: 69 - protein, 21 aa, synthetic construct
RTHLVILGAILLCLGVALTFI

SEQ ID NO: 70 - protein, 22 aa, synthetic construct
RTHLVILGAILLCLGVALTFIF

SEQ ID NO: 71 - protein, 23 aa, synthetic construct
RTHLVILGAILLCLGVALTFIFR

SEQ ID NO: 72 - protein, 24 aa, synthetic construct
RTHLVILGAILLCLGVALTFIFRL

SEQ ID NO: 73 - protein, 25 aa, synthetic construct
RTHLVILGAILLCLGVALTFIFRLR

SEQ ID NO: 74 - protein, 26 aa, synthetic construct
RTHLVILGAILLCLGVALTFIFRLRK

SEQ ID NO: 75 - protein, 27 aa, synthetic construct
RTHLVILGAILLCLGVALTFIFRLRKG

SEQ ID NO: 76 - protein, 29 aa, synthetic construct
RTHLVILGAILLCLGVALTFIFRLRKGRM

SEQ ID NO: 77 - protein, 30 aa, synthetic construct
RTHLVILGAILLCLGVALTFIFRLRKGRMM

SEQ ID NO: 78 - protein, 31 aa, synthetic construct
RTHLVILGAILLCLGVALTFIFRLRKGRMMD

SEQ ID NO: 79 - protein, 22 aa, synthetic construct
ERTHLVILGAILLCLGVALTFI

SEQ ID NO: 80 - protein, 23 aa, synthetic construct
ERTHLVILGAILLCLGVALTFIF

SEQ ID NO: 81 - protein, 24 aa, synthetic construct
ERTHLVILGAILLCLGVALTFIFR

SEQ ID NO: 82 - protein, 25 aa, synthetic construct
ERTHLVILGAILLCLGVALTFIFRL

SEQ ID NO: 83 - protein, 26 aa, synthetic construct
ERTHLVILGAILLCLGVALTFIFRLR

SEQ ID NO: 84 - protein, 27 aa, synthetic construct
ERTHLVILGAILLCLGVALTFIFRLRK

SEQ ID NO: 85 - protein, 28 aa, synthetic construct
ERTHLVILGAILLCLGVALTFIFRLRKG

SEQ ID NO: 86 - protein, 29 aa, synthetic construct
ERTHLVILGAILLCLGVALTFIFRLRKGR

SEQ ID NO: 87 - protein, 30 aa, synthetic construct
ERTHLVILGAILLCLGVALTFIFRLRKGRM

SEQ ID NO: 88 - protein, 31 aa, synthetic construct
ERTHLVILGAILLCLGVALTFIFRLRKGRMM

SEQ ID NO: 89 - protein, 32 aa, synthetic construct
ERTHLVILGAILLCLGVALTFIFRLRKGRMMD

SEQ ID NO: 90 - protein, 23 aa, synthetic construct
NERTHLVILGAILLCLGVALTFI

SEQ ID NO: 91 - protein, 24 aa, synthetic construct
NERTHLVILGAILLCLGVALTFIF

SEQ ID NO: 92 - protein, 25 aa, synthetic construct
NERTHLVILGAILLCLGVALTFIFR

SEQ ID NO: 93 - protein, 26 aa, synthetic construct
NERTHLVILGAILLCLGVALTFIFRL

SEQ ID NO: 94 - protein, 27 aa, synthetic construct
NERTHLVILGAILLCLGVALTFIFRLR

SEQ ID NO: 95 - protein, 28 aa, synthetic construct
NERTHLVILGAILLCLGVALTFIFRLRK

SEQ ID NO: 96 - protein, 29 aa, synthetic construct
NERTHLVILGAILLCLGVALTFIFRLRKG

SEQ ID NO: 97 - protein, 30 aa, synthetic construct
NERTHLVILGAILLCLGVALTFIFRLRKGR

SEQ ID NO: 98 - protein, 31 aa, synthetic construct
NERTHLVILGAILLCLGVALTFIFRLRKGRM

SEQ ID NO: 99 - protein, 32 aa, synthetic construct
NERTHLVILGAILLCLGVALTFIFRLRKGRMM

SEQ ID NO: 100 - protein, 33 aa, synthetic construct
NERTHLVILGAILLCLGVALTFIFRLRKGRMMD

SEQ ID NO: 101 - protein, 273 aa, synthetic construct
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 102 - protein, 21 aa, synthetic construct
SLELQLHQIAALFTVTVPKEL

SEQ ID NO: 103 - protein, 25 aa, synthetic construct
MIFLLLMLSLELQLHQIAALFTVTV

SEQ ID NO: 104 - protein, 9 aa, synthetic construct
LLLMLSLEL

SEQ ID NO: 105 - protein, 322 aa, synthetic construct
MSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 106 - protein, 38 aa, synthetic construct
ISAKDIVYIGLRDVDPGEHYILKTLGIKYFSMTEVDRL

SEQ ID NO: 107 - protein, 32 aa, synthetic construct
ISAKDIVYIGLRDVDPGEHYILKTLGIKYFSM

SEQ ID NO: 108 - protein, 42 aa, synthetic construct
ISAKDIVYIGLRDVDPGEHYILKTLGIKYFSMTEVDRLGIGK

SEQ ID NO: 109 - protein, 20 aa, synthetic construct
AKDIVYIGLRDVDPGEHYIL

SEQ ID NO: 110 - protein, 20 aa, synthetic construct
DVDPGEHYILKTLGIKYFSM

SEQ ID NO: 111 - protein, 20 aa, synthetic construct
KTLGIKYFSMTEVDRLGIGK

SEQ ID NO: 112 - protein, 50 aa, synthetic construct
GFSWVTPCISAKDIVYIGLRDVDPGEHYILKTLGIKYFSMTEVDRLGIGK

SEQ ID NO: 113 - protein, 9 aa, synthetic construct
QLAGKVAEV

SEQ ID NO: 114 - protein, 9 aa, synthetic construct
LVLGGDHSL

SEQ ID NO: 115 - protein, 9 aa, synthetic construct
LLKELKGKI

SEQ ID NO: 116 - protein, 9 aa, synthetic construct
ELKGKIPDV

SEQ ID NO: 117 - protein, 9 aa, synthetic construct
VMEETLSYL

SEQ ID NO: 118 - protein, 9 aa, synthetic construct
HLSFDVDGL

SEQ ID NO: 119 - protein, 9 aa, synthetic construct
SLGKTPEEV

SEQ ID NO: 120 - protein, 9 aa, synthetic construct
IVYIGLRDV

SEQ ID NO: 121 - protein, 9 aa, synthetic construct
GLLSGLDIM

SEQ ID NO: 122 - protein, 9 aa, synthetic construct
DIMEVNPSL

SEQ ID NO: 123 - protein, 10 aa, synthetic construct
LLSGLDIMEV

SEQ ID NO: 124 - protein, 10 aa, synthetic construct
NLHGQPVSFL

SEQ ID NO: 125 - protein, 10 aa, synthetic construct
SLVLGGDHSL

SEQ ID NO: 126 - protein, 10 aa, synthetic construct
YLLGRKKRPI

SEQ ID NO: 127 - protein, 10 aa, synthetic construct
VLGGDHSLAI

SEQ ID NO: 128 - protein, 10 aa, synthetic construct
FLLKELKGKI

SEQ ID NO: 129 - protein, 20 aa, synthetic construct
MSAKSRTIGIIGAPFSKGQP

SEQ ID NO: 130 - protein, 20 aa, synthetic construct
IGAPFSKGQPRGGVEEGPTV

SEQ ID NO: 131 - protein, 20 aa, synthetic construct
RGGVEEGPTVLRKAGLLEKL

SEQ ID NO: 132 - protein, 20 aa, synthetic construct
LRKAGLLEKLKEQECDVKDY

SEQ ID NO: 133 - protein, 20 aa, synthetic construct
KEQECDVKDYGDLPFADIPN

SEQ ID NO: 134 - protein, 20 aa, synthetic construct
GDLPFADIPNDSPFQIVKNP

SEQ ID NO: 135 - protein, 20 aa, synthetic construct
DSPFQIVKNPRSVGKASEQL

SEQ ID NO: 136 - protein, 20 aa, synthetic construct
RSVGKASEQLAGKVAEVKKN

SEQ ID NO: 137 - protein, 20 aa, synthetic construct
AGKVAEVKKNGRISLVLGGD

SEQ ID NO: 138 - protein, 20 aa, synthetic construct
GRISLVLGGDHSLAIGSISG

SEQ ID NO: 139 - protein, 20 aa, synthetic construct
HSLAIGSISGHARVHPDLGV

SEQ ID NO: 140 - protein, 20 aa, synthetic construct
HARVHPDLGVIWVDAHTDIN

SEQ ID NO: 141 - protein, 20 aa, synthetic construct
IWVDAHTDINTPLTTTSGNL

SEQ ID NO: 142 - protein, 20 aa, synthetic construct
TPLTTTSGNLHGQPVSFLLK

SEQ ID NO: 143 - protein, 20 aa, synthetic construct
HGQPVSFLLKELKGKIPDVP

SEQ ID NO: 144 - protein, 20 aa, synthetic construct
ELKGKIPDVPGFSWVTPCIS

SEQ ID NO: 145 - protein, 20 aa, synthetic construct
GFSWVTPCISAKDIVYIGLR

SEQ ID NO: 146 - protein, 20 aa, synthetic construct
TEVDRLGIGKVMEETLSYLL

SEQ ID NO: 147 - protein, 20 aa, synthetic construct
VMEETLSYLLGRKKRPIHLS

SEQ ID NO: 148 - protein, 20 aa, synthetic construct
GRKKRPIHLSFDVDGLDPSF

SEQ ID NO: 149 - protein, 20 aa, synthetic construct
FDVDGLDPSFTPATGTPVVG

SEQ ID NO: 150 - protein, 20 aa, synthetic construct
TPATGTPVVGGLTYREGLYI

SEQ ID NO: 151 - protein, 20 aa, synthetic construct
GLTYREGLYITEEIYKTGLL

SEQ ID NO: 152 - protein, 20 aa, synthetic construct
TEEIYKTGLLSGLDIMEVNP

SEQ ID NO: 153 - protein, 20 aa, synthetic construct
SGLDIMEVNPSLGKTPEEVT

SEQ ID NO: 154 - protein, 20 aa, synthetic construct
SLGKTPEEVTRTVNTAVAIT

SEQ ID NO: 155 - protein, 20 aa, synthetic construct
RTVNTAVAITLACFGLAREG

SEQ ID NO: 156 - protein, 22 aa, synthetic construct
LACFGLAREGNHKPIDYLNPPK

SEQ ID NO: 157 - protein, 20 aa, synthetic construct
GLYITEEIYKTGLLSGLDIM

SEQ ID NO: 158 - protein, 30 aa, synthetic construct
GFSWVTPCISAKDIVYIGLRDVDPGEHYIL

SEQ ID NO: 159 - protein, 354 aa, synthetic construct
MSLRGSLSRLLQTRVHSILKKSVHSVAVIGAPFSQGQKRKGVEHGPAAIREAGLMKRLSSLGCHLKDFGDLSFTPVPKDDLYNNLIVNPRSVGLANQELAEVVSRAVSDGYSCVTLGGDHSLAIGTISGHARHCPDLCVVWVDAHADINTPLTTSSGNLHGQPVSFLLRELQDKVPQLPGFSWIKPCISSASIVYIGLRDVDPPEHFILKNYDIQYFSMRDIDRLGIQKVMERTFDLLIGKRQRPIHLSFDIDAFDPTLAPATGTPVVGGLTYREGMYIAEEIHNTGLLSALDLVEVNPQLATSEEEAKTTANLAVDVIASSFGQTREGGHIVYDQLPTPSSPDESENQARVRI

SEQ ID NO: 160 - protein, 33 aa, synthetic construct
SLRGSLSRLLQTRVHSILKKSVHSVAVIGAPFS

SEQ ID NO: 161 - protein, 29 aa, synthetic construct
SLSRLLQTRVHSILKKSVHSVAVIGAPFS

SEQ ID NO: 162 - protein, 30 aa, synthetic construct
SLRGSLSRLLQTRVHSILKKSVHSVAVIGA

SEQ ID NO: 163 - protein, 9 aa, synthetic construct
NLIVNPRSV

SEQ ID NO: 164 - protein, 9 aa, synthetic construct
GLLSALDLV

SEQ ID NO: 165 - protein, 10 aa, synthetic construct
LLSALDLVEV

SEQ ID NO: 166 - protein, 9 aa, synthetic construct
SLRGSLSRL

SEQ ID NO: 167 - protein, 9 aa, synthetic construct
LLQTRVHSI

SEQ ID NO: 168 - protein, 9 aa, synthetic construct
ILKKSVHSV

SEQ ID NO: 169 - protein, 9 aa, synthetic construct
GLMKRLSSL

SEQ ID NO: 170 - protein, 9 aa, synthetic construct
LANQELAEV

SEQ ID NO: 171 - protein, 9 aa, synthetic construct
VTLGGDHSL

SEQ ID NO: 172 - protein, 10 aa, synthetic construct
FLLRELQDKV

SEQ ID NO: 173 - protein, 9 aa, synthetic construct
LLRELQDKV

SEQ ID NO: 174 - protein, 9 aa, synthetic construct
ELQDKVPQL

SEQ ID NO: 175 - protein, 9 aa, synthetic construct
IVYIGLRDV

SEQ ID NO: 176 - protein, 9 aa, synthetic construct
KVMERTFDL

SEQ ID NO: 177 - protein, 9 aa, synthetic construct
YIAEEIHNT

SEQ ID NO: 178 - protein, 9 aa, synthetic construct
QIVKNPRSV

SEQ ID NO: 179 - protein, 56 aa, synthetic construct
MSLRGSLSRLLQTRVHSILKKSVHSVAVIGAPFSQGQKRKGVEHGPAAIREAGLMK

SEQ ID NO: 180 - protein, 29 aa, synthetic construct
FSWIKPCISSASIVYIGLRDVDPPEHFIL

SEQ ID NO: 181 - protein, 32 aa, synthetic construct
LPGFSWIKPCISSASIVYIGLRDVDPPEHFIL

SEQ ID NO: 182 - protein, 50 aa, synthetic construct
GFSWIKPCISSASIVYIGLRDVDPPEHFILKNYDIQYFSMRDIDRLGIQK

SEQ ID NO: 183 - protein, 20 aa, synthetic construct
MSLRGSLSRLLQTRVHSILK

SEQ ID NO: 184 - protein, 20 aa, synthetic construct
LQTRVHSILKKSVHSVAVIG

SEQ ID NO: 185 - protein, 20 aa, synthetic construct
KSVHSVAVIGAPFSQGQKRK

SEQ ID NO: 186 - protein, 20 aa, synthetic construct
APFSQGQKRKGVEHGPAAIR

SEQ ID NO: 187 - protein, 20 aa, synthetic construct
GVEHGPAAIREAGLMKRLSS

SEQ ID NO: 188 - protein, 20 aa, synthetic construct
EAGLMKRLSSLGCHLKDFGD

SEQ ID NO: 189 - protein, 20 aa, synthetic construct
LGCHLKDFGDLSFTPVPKDD

SEQ ID NO: 190 - protein, 20 aa, synthetic construct
LSFTPVPKDDLYNNLIVNPR

SEQ ID NO: 191 - protein, 20 aa, synthetic construct
LYNNLIVNPRSVGLANQELA

SEQ ID NO: 192 - protein, 20 aa, synthetic construct
SVGLANQELAEVVSRAVSDG

SEQ ID NO: 193 - protein, 20 aa, synthetic construct
EVVSRAVSDGYSCVTLGGDH

SEQ ID NO: 194 - protein, 20 aa, synthetic construct
YSCVTLGGDHSLAIGTISGH

SEQ ID NO: 195 - protein, 20 aa, synthetic construct
SLAIGTISGHARHCPDLCVV

SEQ ID NO: 196 - protein, 20 aa, synthetic construct
ARHCPDLCVVWVDAHADINT

SEQ ID NO: 197 - protein, 20 aa, synthetic construct
WVDAHADINTPLTTSSGNLH

SEQ ID NO: 198 - protein, 20 aa, synthetic construct
PLTTSSGNLHGQPVSFLLRE

SEQ ID NO: 199 - protein, 20 aa, synthetic construct
GQPVSFLLRELQDKVPQLPG

SEQ ID NO: 200 - protein, 20 aa, synthetic construct
LQDKVPQLPGFSWIKPCISS

SEQ ID NO: 201 - protein, 20 aa, synthetic construct
FSWIKPCISSASIVYIGLRD

SEQ ID NO: 202 - protein, 20 aa, synthetic construct
ASIVYIGLRDVDPPEHFILK

SEQ ID NO: 203 - protein, 20 aa, synthetic construct
VDPPEHFILKNYDIQYFSMR

SEQ ID NO: 204 - protein, 20 aa, synthetic construct
NYDIQYFSMRDIDRLGIQKV

SEQ ID NO: 205 - protein, 20 aa, synthetic construct
DIDRLGIQKVMERTFDLLIG

SEQ ID NO: 206 - protein, 20 aa, synthetic construct
MERTFDLLIGKRQRPIHLSF

SEQ ID NO: 207 - protein, 20 aa, synthetic construct
KRQRPIHLSFDIDAFDPTLA

SEQ ID NO: 208 - protein, 20 aa, synthetic construct
DIDAFDPTLAPATGTPVVGG

SEQ ID NO: 209 - protein, 20 aa, synthetic construct
PATGTPVVGGLTYREGMYIA

SEQ ID NO: 210 - protein, 20 aa, synthetic construct
LTYREGMYIAEEIHNTGLLS

SEQ ID NO: 211 - protein, 20 aa, synthetic construct
EEIHNTGLLSALDLVEVNPQ

SEQ ID NO: 212 - protein, 20 aa, synthetic construct
ALDLVEVNPQLATSEEEAKT

SEQ ID NO: 213 - protein, 20 aa, synthetic construct
LATSEEEAKTTANLAVDVIA

SEQ ID NO: 214 - protein, 20 aa, synthetic construct
TANLAVDVIASSFGQTREGG

SEQ ID NO: 215 - protein, 20 aa, synthetic construct
SSFGQTREGGHIVYDQLPTP

SEQ ID NO: 216 - protein, 24 aa, synthetic construct
HIVYDQLPTPSSPDESENQARVRI

SEQ ID NO: 217 - protein, 20 aa, synthetic construct
GFSWIKPCISSASIVYIGLR

SEQ ID NO: 218 - protein, 20 aa, synthetic construct
SASIVYIGLRDVDPPEHFIL

SEQ ID NO: 219 - protein, 20 aa, synthetic construct
DVDPPEHFILKNYDIQYFSM

SEQ ID NO: 220 - protein, 20 aa, synthetic construct
KNYDIQYFSMRDIDRLGIQK

SEQ ID NO: 221 - protein, 406 aa, synthetic construct
MSGCPFLGNNFGYTFKKLPVEGSEEDKSQTGVNRASKGGLIYGNYLHLEKVLNAQELQSETKGNKIHDEHLFIITHQAYELWFKQILWELDSVREIFQNGHVRDERNMLKVVSRMHRVSVILKLLVQQFSILETMTALDFNDFREYLSPASGFQSLQFRLLENKIGVLQNMRVPYNRRHYRDNFKGEENELLLKSEQEKTLLELVEAWLERTPGLEPHGFNFWGKLEKNITRGLEEEFIRIQAKEESEEKEEQVAEFQKQKEVLLSLFDEKRHEHLLSKGERRLSYRALQGALMIYFYREEPRFQVPFQLLTSLMDIDSLMTKWRYNHVCMVHRMLGSKAGTGGSSGYHYLRSTVSDRYKVFVDLFNLSTYLIPRHWIPKMNPTIHKFLYTAEYCDSSYFSSDESD

SEQ ID NO: 222 - protein, 10 aa, synthetic construct
RLLENKIGVL

SEQ ID NO: 223 - protein, 10 aa, synthetic construct
TLLELVEAWL

SEQ ID NO: 224 - protein, 10 aa, synthetic construct
FIITHQAYEL

SEQ ID NO: 225 - protein, 9 aa, synthetic construct
LIYGNYLHL

SEQ ID NO: 226 - protein, 10 aa, synthetic construct
KLLVQQFSIL

SEQ ID NO: 227 - protein, 10 aa, synthetic construct
KIHDEHLFII

SEQ ID NO: 228 - protein, 10 aa, synthetic construct
LLKSEQEKTL

SEQ ID NO: 229 - protein, 9 aa, synthetic construct
QLLTSLMDI

SEQ ID NO: 230 - protein, 9 aa, synthetic construct
QILWELDSV

SEQ ID NO: 231 - protein, 9 aa, synthetic construct
SILETMTAL

SEQ ID NO: 232 - protein, 9 aa, synthetic construct
LLSKGERRL

SEQ ID NO: 233 - protein, 9 aa, synthetic construct
DLFNLSTYL

SEQ ID NO: 234 - protein, 10 aa, synthetic construct
KLEKNITRGL

SEQ ID NO: 235 - protein, 10 aa, synthetic construct
LIPRHWIPKM

SEQ ID NO: 236 - protein, 10 aa, synthetic construct
KMNPTIHKFL

SEQ ID NO: 237 - protein, 20 aa, synthetic construct
VSVILKLLVQQFSILETMTA

SEQ ID NO: 238 - protein, 20 aa, synthetic construct
RFQVPFQLLTSLMDIDSLMT

SEQ ID NO: 239 - protein, 390 aa, synthetic construct
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 240 - protein, 29 aa, synthetic construct
MPPSGLRLLLLLLPLLWLLVLTPGRPAAG

SEQ ID NO: 241 - protein, 249 aa, synthetic construct
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRR

SEQ ID NO: 242 - protein, 112 aa, synthetic construct
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 243 - protein, 20 aa, synthetic construct
REAVPEPVLLSRAELRLLRL

SEQ ID NO: 244 - protein, 10 aa, synthetic construct
VLLSRAELRL

SEQ ID NO: 245 - protein, 25 aa, synthetic construct
NTSELREAVPEPVLLSRAELRLLRL

SEQ ID NO: 246 - protein, 25 aa, synthetic construct
REAVPEPVLLSRAELRLLRLKLKVE

SEQ ID NO: 247 - protein, 30 aa, synthetic construct
NTSELREAVPEPVLLSRAELRLLRLKLKVE

SEQ ID NO: 248 - protein, 20 aa, synthetic construct
MPPSGLRLLLLLLPLLWLLV

SEQ ID NO: 249 - protein, 10 aa, synthetic construct
LLLLLPLLWL

SEQ ID NO: 250 - protein, 20 aa, synthetic construct
LLLPLLWLLVLTPGRPAAGL

SEQ ID NO: 251 - protein, 25 aa, synthetic construct
LLLPLLWLLVLTPGRPAAGLSTCKT

SEQ ID NO: 252 - protein, 25 aa, synthetic construct
LRLLLLLLPLLWLLVLTPGRPAAGL

SEQ ID NO: 253 - protein, 30 aa, synthetic construct
LRLLLLLLPLLWLLVLTPGRPAAGLSTCKT

SEQ ID NO: 254 - protein, 20 aa, synthetic construct
LTPGRPAAGLSTCKTIDMEL

SEQ ID NO: 255 - protein, 20 aa, synthetic construct
STCKTIDMELVKRKRIEAIR

SEQ ID NO: 256 - protein, 20 aa, synthetic construct
VKRKRIEAIRGQILSKLRLA

SEQ ID NO: 257 - protein, 25 aa, synthetic construct
IDMELVKRKRIEAIRGQILSKLRLA

SEQ ID NO: 258 - protein, 25 aa, synthetic construct
VKRKRIEAIRGQILSKLRLASPPSQ

SEQ ID NO: 259 - protein, 30 aa, synthetic construct
IDMELVKRKRIEAIRGQILSKLRLASPPSQ

SEQ ID NO: 260 - protein, 20 aa, synthetic construct
GQILSKLRLASPPSQGEVPP

SEQ ID NO: 261 - protein, 20 aa, synthetic construct
SPPSQGEVPPGPLPEAVLAL

SEQ ID NO: 262 - protein, 20 aa, synthetic construct
GPLPEAVLALYNSTRDRVAG

SEQ ID NO: 263 - protein, 20 aa, synthetic construct
YNSTRDRVAGESAEPEPEPE

SEQ ID NO: 264 - protein, 20 aa, synthetic construct
ESAEPEPEPEADYYAKEVTR

SEQ ID NO: 265 - protein, 20 aa, synthetic construct
ADYYAKEVTRVLMVETHNEI

SEQ ID NO: 266 - protein, 20 aa, synthetic construct
VLMVETHNEIYDKFKQSTHS

SEQ ID NO: 267 - protein, 20 aa, synthetic construct
YDKFKQSTHSIYMFFNTSEL

SEQ ID NO: 268 - protein, 25 aa, synthetic construct
THNEIYDKFKQSTHSIYMFFNTSEL

SEQ ID NO: 269 - protein, 25 aa, synthetic construct
YDKFKQSTHSIYMFFNTSELREAVP

SEQ ID NO: 270 - protein, 30 aa, synthetic construct
THNEIYDKFKQSTHSIYMFFNTSELREAVP

SEQ ID NO: 271 - protein, 20 aa, synthetic construct
IYMFFNTSELREAVPEPVLL

SEQ ID NO: 272 - RNA, 63 nt, synthetic construct
  source 1..63: mol_type = other RNA
gacaccctgctgaaggccctgctggagatcgccagctgcctggagaaggccctgcaggtgttc

SEQ ID NO: 273 - RNA, 57 nt, synthetic construct
  source 1..57: mol_type = other RNA
ttcatgacctactggcacctgctgaacgccttcaccgtgaccgtgcccaaggacctg

SEQ ID NO: 274 - RNA, 63 nt, synthetic construct
  source 1..63: mol_type = other RNA
agcctggagctgcagctgcaccagatcgccgccctgttcaccgtgaccgtgcccaaggagctg

SEQ ID NO: 275 - RNA, 75 nt, synthetic construct
  source 1..75: mol_type = other RNA
atgatcttcctgctgctgatgctgagcctggagctgcagctgcaccagatcgccgccctgttcaccgtgaccgtg

SEQ ID NO: 276 - RNA, 27 nt, synthetic construct
  source 1..27: mol_type = other RNA
ctgctgctgatgctgagcctggagctg

SEQ ID NO: 277 - RNA, 114 nt, synthetic construct
  source 1..114: mol_type = other RNA
atcagcgccaaggacatcgtgtacatcggcctgagggacgtggaccccggcgagcactacatcctgaagaccctgggcatcaagtacttcagcatgaccgaggtggacaggctg

SEQ ID NO: 278 - RNA, 96 nt, synthetic construct
  source 1..96: mol_type = other RNA
atcagcgccaaggacatcgtgtacatcggcctgagggacgtggaccccggcgagcactacatcctgaagaccctgggcatcaagtacttcagcatg

SEQ ID NO: 279 - RNA, 126 nt, synthetic construct
  source 1..126: mol_type = other RNA
atcagcgccaaggacatcgtgtacatcggcctgagggacgtggaccccggcgagcactacatcctgaagaccctgggcatcaagtacttcagcatgaccgaggtggacaggctgggcatcggcaag

SEQ ID NO: 280 - RNA, 99 nt, synthetic construct
  source 1..99: mol_type = other RNA
agcctgaggggcagcctgagcaggctgctgcagaccagggtgcacagcatcctgaagaagagcgtgcacagcgtggccgtgatcggcgcccccttcagc

SEQ ID NO: 281 - RNA, 60 nt, synthetic construct
  source 1..60: mol_type = other RNA
agggaggccgtgcccgagcccgtgctgctgagcagggccgagctgaggctgctgaggctg

SEQ ID NO: 282 - DNA, 63 bp, synthetic construct
  source 1..63: mol_type = other DNA
gacaccctgctgaaggccctgctggagatcgccagctgcctggagaaggccctgcaggtgttc

SEQ ID NO: 283 - DNA, 57 bp, synthetic construct
  source 1..57: mol_type = other DNA
ttcatgacctactggcacctgctgaacgccttcaccgtgaccgtgcccaaggacctg

SEQ ID NO: 284 - DNA, 63 bp, synthetic construct
  source 1..63: mol_type = other DNA
agcctggagctgcagctgcaccagatcgccgccctgttcaccgtgaccgtgcccaaggagctg

SEQ ID NO: 285 - DNA, 75 bp, synthetic construct
  source 1..75: mol_type = other DNA
atgatcttcctgctgctgatgctgagcctggagctgcagctgcaccagatcgccgccctgttcaccgtgaccgtg

SEQ ID NO: 286 - DNA, 27 bp, synthetic construct
  source 1..27: mol_type = other DNA
ctgctgctgatgctgagcctggagctg

SEQ ID NO: 287 - DNA, 114 bp, synthetic construct
  source 1..114: mol_type = other DNA
atcagcgccaaggacatcgtgtacatcggcctgagggacgtggaccccggcgagcactacatcctgaagaccctgggcatcaagtacttcagcatgaccgaggtggacaggctg

SEQ ID NO: 288 - DNA, 96 bp, synthetic construct
  source 1..96: mol_type = other DNA
atcagcgccaaggacatcgtgtacatcggcctgagggacgtggaccccggcgagcactacatcctgaagaccctgggcatcaagtacttcagcatg

SEQ ID NO: 289 - DNA, 126 bp, synthetic construct
  source 1..126: mol_type = other DNA
atcagcgccaaggacatcgtgtacatcggcctgagggacgtggaccccggcgagcactacatcctgaagaccctgggcatcaagtacttcagcatgaccgaggtggacaggctgggcatcggcaag

SEQ ID NO: 290 - DNA, 99 bp, synthetic construct
  source 1..99: mol_type = other DNA
agcctgaggggcagcctgagcaggctgctgcagaccagggtgcacagcatcctgaagaagagcgtgcacagcgtggccgtgatcggcgcccccttcagc

SEQ ID NO: 291 - DNA, 60 bp, synthetic construct
  source 1..60: mol_type = other DNA
agggaggccgtgcccgagcccgtgctgctgagcagggccgagctgaggctgctgaggctg